(19) United States
(12) Patent Application Publication
(10) Pub. No.: US 2026/0309694 A1
(43) Pub. Date: Oct. 8, 2026

(54) METHOD

(71) Applicant: Oxford Nanopore Technologies PLC, Oxford (GB)

(72) Inventors: Lakmal Nishantha Jayasinghe, Oxford (GB); John Joseph Kilgour, Oxford (GB); Neil Roger Wood, Oxford (GB)

(73) Assignee: Oxford Nanopore Technologies PLC, Oxford (GB)

(21) Appl. No.: 19/646,420

(22) Filed: Apr. 13, 2026

Related U.S. Application Data
(63) Continuation of application No. 17/693,922, filed on Mar. 14, 2022, now Pat. No. 12,644,879.
Application No. 17/693,922 is a continuation of application No. 15/551,884, filed on Feb. 17, 2016, now Pat. No. 11,307,192.

(30) Foreign Application Priority Data
Feb. 19, 2015 (GB) 1502810.3

(51) Int. Cl. G01N 33/487; B82B 3/00; C12Q 1/6869; G16B 30/00

(52) U.S. Cl. CPC G01N 33/48721; B82B 3/00; B82B 3/0047; C12Q 1/6869; G16B 30/00; C12Q 2565/631

(57) ABSTRACT

The invention relates to a novel method of producing hetero-oligomeric pores. The invention also relates to hetero-oligomeric pores produced using the method and polynucleotide characterisation using the hetero-oligomeric pores.

### RELATED APPLICATIONS

This application is a continuation of U.S. application Ser. No. 17/693,922, filed Mar. 14, 2022, which is a is a continuation of U.S. application Ser. No. 15/551,884, filed Aug. 17, 2017, which is a national stage application under 35 U.S.C. § 371 of PCT International Application No. PCT/GB2016/050391, filed Feb. 17, 2016, which claims foreign priority benefits under 35 U.S.C. § 119(a)-(d) or 35 U.S.C. § 365(b) of British application number 1502810.3, filed Feb. 19, 2015, the contents of each of which are incorporated herein by reference in their entireties.

### REFERENCE OF ELECTRONIC SEQUENCE LISTING

The contents of the electronic sequence listing (0036670059US02-SEQ-LJG.xml; Size: 53,059 bytes; and Date of Creation: Apr. 13, 2026) are herein incorporated by reference in their entirety.

### FIELD OF THE INVENTION

The invention relates to a novel method of producing hetero-oligomeric pores. The invention also relates to hetero-oligomeric pores produced using the method and polynucleotide characterisation using the hetero-oligomeric pores.

### BACKGROUND OF THE INVENTION

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the strand sequencing method, a single polynucleotide strand is passed through the pore and the identities of the nucleotides are derived. Strand sequencing can involve the use of a polynucleotide binding protein to control the movement of the polynucleotide through the pore.

The different forms of Msp are porins from Mycobacterium smegmatis. MspA is a 157 kDa octameric porin from Mycobacterium smegmatis. Wild-type MspA does not interact with DNA in a manner that allows the DNA to be characterised or sequenced. The structure of MspA and the modifications required for it to interact with and characterise DNA have been well documented (Butler, 2007, Nanopore Analysis of Nucleic Acids, Doctor of Philosophy Dissertation, University of Washington; Gundlach, Proc Natl Acad Sci USA. 2010 Sep. 14; 107 (37): 16060-5. Epub 2010 Aug. 26; and International Application No. PCT/GB2012/050301 (published as WO/2012/107778). Negative charges, such as those at positions 90, 91 and 93, are typically removed from the pore to make it neutral.

### SUMMARY OF THE INVENTION

The inventors have surprisingly demonstrated that hetero-oligomeric pores comprising two different monomers in a specific stoichiometric ratio can be produced in a single cell by differentially expressing the two different monomers.

Accordingly, the invention provides a method for producing a hetero-oligomeric pore comprising two different monomers in a specific stoichiometric ratio, comprising: (a) transfecting or transforming a cell with the first different monomer in a first inducible vector; (b) transfecting or transforming the cell with the second different monomer in a second inducible vector; and (c) inducing the first and second inducible vectors such that the cell produces the hetero-oligomeric pore comprising the first and second different monomers in the specific stoichiometric ratio.

The invention also provides:
a hetero-oligomeric pore produced using a method of the invention;
a method of characterising a target polynucleotide, comprising:
a) contacting the polynucleotide with a hetero-oligomeric pore of the invention such that the polynucleotide moves through the pore; and
b) taking one or more measurements as the polynucleotide moves with respect to the pore, wherein the measurements are indicative of one or more characteristics of the polynucleotide, and thereby characterising the target polynucleotide;
a kit for characterising a target polynucleotide comprising (a) a hetero-oligomeric pore of the invention and (b) the components of a membrane;
an apparatus for characterising target polynucleotides in a sample, comprising (a) a plurality of hetero-oligomeric pores of the invention and (b) a plurality of membranes;
a method of characterising a target polynucleotide, comprising:
a) contacting the polynucleotide with a hetero-oligomeric pore of the invention, a polymerase and labelled nucleotides such that phosphate labelled species are sequentially added to the target polynucleotide by the polymerase, wherein the phosphate species contain a label specific for each nucleotide; and
b) detecting the phosphate labelled species using the pore and thereby characterising the polynucleotide;
a method of forming a sensor for characterising a target polynucleotide, comprising forming a complex between a hetero-oligomeric pore of the invention and a polynucleotide binding protein and thereby forming a sensor for characterising the target polynucleotide; and
a sensor for characterising a target polynucleotide, comprising a complex between a hetero-oligomeric pore of the invention and a polynucleotide binding protein.

### DESCRIPTION OF THE FIGURES

FIG. 1 shows a 10% TGX gel with bands visualised with coomassie stain. Lane 1 corresponds to a protein molecular weight marker. The numbers along the side of the gel correspond to kDa. Lane 2 corresponds to purified MspA 2. Lane 3 corresponds to MspA 2 after heating at 85° C. Lane 4 corresponds to MspA 2 after heating to 100° C. in 50% DMSO. Lane 5 corresponds to purified MspA—((Del-L74/G75/D118/L119) D56N/E59R/L88N/D90N/D91N/Q126R/D134R/E139K) 8=MspA 6 (homopolymeric nanopore as a reference). Lane 6 corresponds to MspA 6 after heating to 100° C. in 50% DMSO (under these conditions the oligomeric pore can be broken down into its constituent monomer subunits). Band A corresponds to oligomerised MspA homo/heteropores comprising 8 monomer units. Band B corresponds to the monomer unit of MspA 2 which contained the BasTL-H6 attached. Band C corresponds to the MspA monomer units which did not contain a BasTL-H6 attached.

FIG. 2 shows a 7.5% Tris HCl gel which compares oligomerisation of MspA—(G75S/G77S/L88N/D90N/D91N/D93N/D118R/Q126R/D134R/E139K) (SEQ ID NO: 2 with the following mutations G75S/G77S/L88N/D90N/D91N/D93N/D118R/Q126R/D134R/E139K) with or without a BasTL tag of varying length. Lane A corresponded to homo-oligomer MspA—(G75S/G77S/L88N/D90N/D91N/D93N/D118R/Q126R/D134R/E139K) 8 which had no tags attached to any of the monomers. Lane B corresponded to a 2:1 mixture of MspA—(G75S/G77S/L88N/D90N/D91N/D93N/D118R/Q126R/D134R/E139K) monomer and MspA—(G75S/G77S/L88N/D90N/D91N/D93N/D118R/Q126R/D134R/E139K/BasTL) monomer. Lane C corresponded to homo-oligomer MspA—(G75S/G77S/L88N/D90N/D91N/D93N/D118R/Q126R/D134R/E139K/BasTL (minus 20 aa)) 8. Lane D corresponded to a 2:1 mixture of MspA—(G75S/G77S/L88N/D90N/D91N/D93N/D118R/Q126R/D134R/E139K) monomer and MspA—(G75S/G77S/L88N/D90N/D91N/D93N/D118R/Q126R/D134R/E139K/BasTL (minus 20 aa)) monomer. Lane E corresponded to homo-oligomer MspA—(G75S/G77S/L88N/D90N/D91N/D93N/D118R/Q126R/D134R/E139K/BasTL (minus 40 aa)) 8. Lane F corresponded to a 2:1 mixture of MspA—(G75S/G77S/L88N/D90N/D91N/D93N/D118R/Q126R/D134R/E139K) monomer and MspA—(G75S/G77S/L88N/D90N/D91N/D93N/D118R/Q126R/D134R/E139K/BasTL (minus 40 aa)) monomer. The ratios quoted next to the gel bands correspond to the ratio of tagged monomer:non-tagged monomer.

FIG. 3 shows 5% Tris HCl gel which compares oligomerisation of MspA—(G75S/G77S/L88N/D90N/D91N/D93N/D118R/Q126R/D134R/E139K) monomers when a variety of different tags were attached to the C terminus. Lane A corresponded to homo-oligomer MspA—(G75S/G77S/L88N/D90N/D91N/D93N/D118R/Q126R/D134R/E139K) 8 which had no tags attached to any of the monomers. Lane B corresponded to homo-oligomer MspA—(G75S/G77S/L88N/D90N/D91N/D93N/D118R/Q126R/D134R/E139K) 8 which had been heat treated at 85° C. for 15 minutes. Lane C corresponded to the attempt to oligomerise the homo-oligomer MspA—(G75S/G77S/L88N/D90N/D91N/D93N/D118R/Q126R/D134R/E139K/D10H6) 8 (SEQ ID NO: 2 with the following mutations G75S/G77S/L88N/D90N/D91N/D93N/D118R/Q126R/D134R/E139K where there is a D10H6 tag attached at the C terminus). Lane D corresponded to the attempt to oligomerise the homo-oligomer MspA—(G75S/G77S/L88N/D90N/D91N/D93N/D118R/Q126R/D134R/E139K/D10H6) 8 (SEQ ID NO: 2 with the following mutations G75S/G77S/L88N/D90N/D91N/D93N/D118R/Q126R/D134R/E139K where there is a D10H6 tag attached at the C terminus) which had been heat treated at 85° C. for 15 minutes. Lane E corresponded to the attempt to oligomerise the homo-oligomer MspA—(G75S/G77S/L88N/D90N/D91N/D93N/D118R/Q126R/D134R/E139K/R8) 8 (SEQ ID NO: 2 with the following mutations G75S/G77S/L88N/D90N/D91N/D93N/D118R/Q126R/D134R/E139K where there is a R8 tag attached at the C terminus). Lane F corresponded to the attempt to oligomerise the homo-oligomer MspA—(G75S/G77S/L88N/D90N/D91N/D93N/D118R/Q126R/D134R/E139K/R8) 8 (SEQ ID NO: 2 with the following mutations G75S/G77S/L88N/D90N/D91N/D93N/D118R/Q126R/D134R/E139K where there is a R8 tag attached at the C terminus) which had been heat treated at 85° C. for 15 minutes. Lane G corresponded to the attempt to oligomerise the homo-oligomer MspA—(G75S/G77S/L88N/D90N/D91N/D93N/D118R/Q126R/D134R/E139K/R8H6) 8 (SEQ ID NO: 2 with the following mutations 5 G75S/G77S/L88N/D90N/D91N/D93N/D118R/Q126R/D134R/E139K where there is a R8H6 tag attached at the C terminus). Lane H corresponded to the attempt to oligomerise the homo-oligomer MspA—(G75S/G77S/L88N/D90N/D91N/D93N/D118R/Q126R/D134R/E139K/R8H6) 8 (SEQ ID NO: 2 with the following mutations G75S/G77S/L88N/D90N/D91N/D93N/D118R/Q126R/D134R/E139K where there is a R8H6 tag attached at the C terminus) which had been heat treated at 85° C. for 15 minutes. White dots indicate the MspA homo-oligomer formed in each lane.

### DESCRIPTION OF THE SEQUENCE LISTING

SEQ ID NO: 1 shows the codon optimised polynucleotide sequence encoding the wild-type MspA monomer. This mutant lacks the signal sequence.

SEQ ID NO: 2 shows the amino acid sequence of the mature form of the wild-type MspA monomer. This mutant lacks the signal sequence.

SEQ ID NO: 3 shows the polynucleotide sequence encoding one monomer of α-hemolysin-E111N/K147N (α-HL-NN; Stoddart et al., PNAS, 2009; 106 (19): 7702-7707).

SEQ ID NO: 4 shows the amino acid sequence of one monomer of α-HL-NN.

SEQ ID NOs: 5 to 7 show the amino acid sequences of MspB, C and D.

SEQ ID NO: 8 shows the polynucleotide sequence encoding the Phi29 DNA polymerase.

SEQ ID NO: 9 shows the amino acid sequence of the Phi29 DNA polymerase.

SEQ ID NO: 10 shows the codon optimised polynucleotide sequence derived from the sbcB gene from E. coli. It encodes the exonuclease I enzyme (EcoExo I) from E. coli.

SEQ ID NO: 11 shows the amino acid sequence of exonuclease I enzyme (EcoExo I) from E. coli.

SEQ ID NO: 12 shows the codon optimised polynucleotide sequence derived from the xthA gene from E. coli. It encodes the exonuclease III enzyme from E. coli.

SEQ ID NO: 13 shows the amino acid sequence of the exonuclease III enzyme from E. coli. This enzyme performs distributive digestion of 5′ monophosphate nucleosides from one strand of double stranded DNA (dsDNA) in a 3′-5′ direction. Enzyme initiation on a strand requires a 5′ overhang of approximately 4 nucleotides.

SEQ ID NO: 14 shows the codon optimised polynucleotide sequence derived from the recJ gene from T. thermophilus. It encodes the RecJ enzyme from T. thermophilus (TthRecJ-cd).

SEQ ID NO: 15 shows the amino acid sequence of the RecJ enzyme from T. thermophilus (TthRecJ-cd). This enzyme performs processive digestion of 5′ monophosphate nucleosides from ssDNA in a 5′-3′ direction. Enzyme initiation on a strand requires at least 4 nucleotides.

SEQ ID NO: 16 shows the codon optimised polynucleotide sequence derived from the bacteriophage lambda exo (redX) gene. It encodes the bacteriophage lambda exonuclease.

SEQ ID NO: 17 shows the amino acid sequence of the bacteriophage lambda exonuclease. The sequence is one of three identical subunits that assemble into a trimer. The enzyme performs highly processive digestion of nucleotides from one strand of dsDNA, in a 5′-3′direction (http://www.neb.com/nebecomm/products/productM0262.asp). Enzyme initiation on a strand preferentially requires a 5′ overhang of approximately 4 nucleotides with a 5′ phosphate.

SEQ ID NO: 18 shows the amino acid sequence of Hel308 Mbu.

SEQ ID NO: 19 shows the amino acid sequence of Hel308 Csy.

SEQ ID NO: 20 shows the amino acid sequence of Hel308 Tga.

SEQ ID NO: 21 shows the amino acid sequence of Hel308 Mhu.

SEQ ID NO: 22 shows the amino acid sequence of TraI Eco.

SEQ ID NO: 23 shows the amino acid sequence of XPD Mbu.

SEQ ID NO: 24 shows the amino acid sequence of Dda 1993.

SEQ ID NO: 25 shows the amino acid sequence of Trwc Cba.

SEQ ID NO: 26 shows the amino acid sequence of the BasTL. This sequence is attached at the C terminus of the MspA monomers.

SEQ ID NO: 27 shows a polynucleotide encoding sequence used in Example 1 and 2.

SEQ ID NO: 28 shows a polynucleotide encoding sequence used in Example 1 and 2.

SEQ ID NO: 29 shows a polynucleotide encoding sequence used in Example 3.

SEQ ID NO: 30 shows a polynucleotide encoding sequence used in Example 3.

SEQ ID NO: 31 shows the amino acid sequence of the lysenin monomer.

SEQ ID NO: 32 shows the amino acid sequence of the mature form of the wild-type CsgG monomer from Escherchia coli Str. K-12 substr. MC4100. This monomer lacks the signal sequence. The abbreviation used for this CsgG=CsgG-Eco.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### Method of the Invention

### Hetero-Oligomeric Pore

The invention provides a method for producing a hetero-oligomeric pore. The hetero-oligomeric pore contains sufficient monomers to form the pore. The monomers may be of any type. The pore typically comprises at least 3, at least 4, at least 5, at least 6, at least 7, at least 8, at least 9, at least 10 monomers, at least 11 monomers, at least 12 monomers, at least 13 monomers or at least 14 monomers, such as 3, 4, 5, 6, 7, 8, 9, 10, 11, 12, 13 or 14 monomers. The pore preferably comprises seven, eight or nine monomers.

The pore is hetero-oligomeric because it comprises two different monomers. The pore may be hetero-oligomeric because it comprises at least two different monomers. The two monomers may be different in any way. The second different monomer is typically different from the first different monomer on the basis of its amino acid sequence. The second different monomer may be different from the first different monomer on the basis of 1, 2, 3, 4, 5, 10, 15, 20, 30, 40 or more amino acid differences. The second different monomer may be modified with a tag as discussed below.

The hetero-oligomeric pore is typically derived from or based on a transmembrane protein pore. The hetero-oligomeric pore is typically a variant of a transmembrane protein pore. The hetero-oligomeric pore is typically a variant of a transmembrane protein pore in which one or more of the monomers in the pore has been modified such that it is different from the others. The hetero-oligomeric pore typically comprises monomers from a transmembrane protein pore, such as MspA, MspB, MspC or MspD, in which one or more of the monomers in the pore has been modified such that it is different from the others.

The hetero-oligomeric pore typically does not comprise two different monomers each from two different transmembrane protein pores. In particular, the hetero-oligomeric pore typically does not comprise a first monomer from MspA and a second monomer from α-hemolysin. The hetero-oligomeric pore may comprise two different monomers each from two different Msp pores, such as two of MspA, MspB, MspC and MspD.

The hetero-oligomeric pore may be derived from or based on any transmembrane protein pore. A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

Any transmembrane protein pore may be used in the invention. The transmembrane protein pore is typically a collection of polypeptides, i.e. an oligomer, that are arranged around a central axis to form a channel. The channel permits hydrated ions, nucleotides or polynucleotides, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analytes such as nucleotides or polynucleotides to flow from one side of the membrane, such as a triblock copolymer membrane, to the other. The transmembrane protein pore typically allows a polynucleotide, such as DNA or RNA, to be moved through the pore.

The transmembrane protein pore is an oligomer. The transmembrane protein pore is preferably made up of several repeating subunits, such as least 3, at least 4, at least 5, at least 6, at least 7, at least 8, at least 9, at least 10 monomers, at least 11 monomers, at least 12 monomers, at least 13 monomers or at least 14 monomers, such as 3, 4, 5, 6, 7, 8, 9, 10, 11, 12, 13 or 14 monomers. The pore is preferably a trimeric, tetrameric, pentameric, hexameric, heptameric, octameric or nonameric pore. The naturally-occurring transmembrane protein pore may be a homo-oligomer or a hetero-oligomer.

The transmembrane protein pore typically comprises a barrel or channel through which the ions may flow. The subunits of the pore typically surround a central axis and contribute strands to a transmembrane β barrel or channel or a transmembrane α-helix bundle or channel.

The barrel or channel of the transmembrane protein pore typically comprises amino acids that facilitate interaction with analyte, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β pore forming toxins, such as α-hemolysin, anthrax toxin, NetB, CytK and leukocidins, and outer membrane proteins/porins of bacteria, such as Mycobacterium smegmatis porin (Msp), for example MspA, MspB, MspC or MspD, NfpAB pore from Nocardia farcinica, outer membrane porin F (OmpF), outer membrane porin G (OmpG), outer membrane phospholipase A and Neisseria autotransporter lipoprotein (NalP), and other pores, such as lysenin, CsgG and FRAC. α-helix bundle pores comprise a barrel or channel that is formed from α-helices. Suitable α-helix bundle pores include, but are not limited to, inner membrane proteins and α outer membrane proteins, such as WZA and ClyA toxin. The transmembrane pore may be derived from lysenin. Suitable pores derived from lysenin are disclosed in International Application No. PCT/GB2013/050667 (published as WO 2013/153359). The transmembrane pore may be derived from CsgG. Suitable pores derived from CsgG are disclosed in International Application No. PCT/EP2015/069965. The transmembrane pore may be derived from Msp, such as MspA, or from α-hemolysin (α-HL).

The hetero-oligomeric pore may be derived from or based on MspA or a variant thereof, i.e. derived from a transmembrane pore comprising seven or more monomers comprising the sequence shown in SEQ ID NO: 2 or a variant thereof. The hetero-oligomeric pore is more preferably derived from a pore which comprises 8 or 9 monomers comprising the sequence shown in SEQ ID NO: 2 or a variant thereof. The first different monomer preferably comprises the sequence shown in SEQ ID NO: 2 or a variant thereof. The second different monomer preferably comprises the sequence shown in SEQ ID NO: 2 or a variant thereof. Most preferably, the first and second different monomers comprise different variants of the sequence shown in SEQ ID NO: 2. SEQ ID NO: 2 and variants thereof are discussed in more detail below.

The hetero-oligomeric pore may also be derived from or based on α-hemolysin (α-HL). The wild type α-HL pore is formed of seven identical monomers or subunits (i.e. it is heptameric). The sequence of one monomer or subunit of α-hemolysin-NN is shown in SEQ ID NO: 4. The hetero-oligomeric pore may be derived from a transmembrane protein pore comprising seven monomers each comprising the sequence shown in SEQ ID NO: 4 or a variant thereof. The hetero-oligomeric pore produced in accordance with the invention may comprise two different monomers each the sequence shown in SEQ ID NO: 4 or a variant thereof. The first different monomer preferably comprises the sequence shown in SEQ ID NO: 4 or a variant thereof. The second different monomer preferably comprises the sequence shown in SEQ ID NO: 4 or a variant thereof. Most preferably, the first and second different monomers comprise different variants of the sequence shown in SEQ ID NO: 4.

Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290 and 294 of SEQ ID NO: 4 form loop regions. Residues 113 and 147 of SEQ ID NO: 4 form part of a constriction within the barrel or channel of α-HL.

A variant of SEQ ID NO: 4 is a protein that has an amino acid sequence which varies from that of SEQ ID NO: 4 and which retains its pore forming ability. The ability of a variant to form a pore can be assayed using any method known in the art. For instance, the variant may be inserted into an amphiphilic layer, such as a triblock copolymer membrane, along with other appropriate subunits and its ability to oligomerise to form a pore may be determined. Methods are known in the art for inserting subunits into amphiphilic layers, such as triblock copolymer membranes.

The variant may include modifications that facilitate covalent attachment. The variant preferably comprises one or more reactive cysteine residues that facilitate attachment, for instance to other monomers. For instance, the variant may include a cysteine at one or more of positions 8, 9, 17, 18, 19, 44, 45, 50, 51, 237, 239 and 287 and/or on the amino or carboxy terminus of SEQ ID NO: 4. Preferred variants comprise a substitution of the residue at position 8, 9, 17, 237, 239 and 287 of SEQ ID NO: 4 with cysteine (A8C, T9C, N17C, K237C, S239C or E287C). The variant is preferably any one of the variants described in International Application No. PCT/GB09/001690 (published as WO 2010/004273), PCT/GB09/001679 (published as WO 2010/004265) or PCT/GB 10/000133 (published as WO 2010/086603).

The variant may also include modifications that facilitate any interaction with nucleotides.

The variant may be a naturally occurring variant which is expressed naturally by an organism, for instance by a Staphylococcus bacterium. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 4, a variant will preferably be at least 50% homologous to that sequence based on amino acid similarity or identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid similarity or identity to the amino acid sequence of SEQ ID NO: 4 over the entire sequence. There may be at least 80%, for example at least 85%, 90% or 95%, amino acid similarity or identity over a stretch of 200 or more, for example 230, 250, 270 or 280 or more, contiguous amino acids (“hard homology”). Homology can be determined as discussed below.

The hetero-oligomeric pore may also be derived from or based on lysenin. The wild type α-HL pore is formed of at least 7, at least 8, at least 9 or at least 10, preferably nine, identical monomers or subunits (i.e. it is heptameric). The sequence of one monomer or subunit of lysenin is shown in SEQ ID NO: 31. The hetero-oligomeric pore may be derived from a transmembrane protein pore comprising at least 7, at least 8, at least 9 or at least 1, preferably nine, monomers each comprising the sequence shown in SEQ ID NO: 31 or a variant thereof. The hetero-oligomeric pore produced in accordance with the invention may comprise two different monomers each the sequence shown in SEQ ID NO: 31 or a variant thereof. The first different monomer preferably comprises the sequence shown in SEQ ID NO: 31 or a variant thereof. The second different monomer preferably comprises the sequence shown in SEQ ID NO: 31 or a variant thereof. Most preferably, the first and second different monomers comprise different variants of the sequence shown in SEQ ID NO: 31.

The variant preferably comprises one or more modifications from about position 44 to about position 126 of SEQ ID NO: 2 which alter the ability of the monomer to interact with a polynucleotide. Such modifications are disclosed in International Application No. PCT/GB2013/050667 (published as WO2013153359).

The ability of the monomer to interact with a polynucleotide can be determined using methods that are well-known in the art. The monomer may interact with a polynucleotide in any way, e.g. by non-covalent interactions, such as hydrophobic interactions, hydrogen bonding, Van der Waal's forces, pi (π)-cation interactions or electrostatic forces. For instance, the ability of the region to bind to a polynucleotide can be measured using a conventional binding assay. Suitable assays include, but are not limited to, fluorescence-based binding assays, nuclear magnetic resonance (NMR), Isothermal Titration calorimetry (ITC) or Electron spin resonance (ESR) spectroscopy. Alternatively, the ability of a pore comprising one or more of the mutant monomers to interact with a polynucleotide can be determined using any of the methods discussed above or below.

The one or more modifications are within the region from about position 44 to about position 126 of SEQ ID NO: 2. The one or more modifications are preferably within any one of the following regions: from about position 40 to about position 125, from about position 50 to about position 120, from about position 60 to about position 110 and from about position 70 to about position 100. If the one or more modifications are being made to improve polynucleotide capture, they are more preferably made within any one of the following regions: from about position 44 to about position 103, from about position 68 to about position 103, from about position 84 to about position 103, from about position 44 to about position 97, from about position 68 to about position 97 or from about position 84 to about position 97. If the one or more modifications are being made to improve polynucleotide recognition or discrimination, they are more preferably made within any one of the following regions: from about position 44 to about position 109, from about position 44 to about position 97 or from about position 48 to about position 88. The region is preferably from about position 44 to about position 67 of SEQ ID NO: 2.

If the one or more modifications are intended improve polynucleotide recognition or discrimination, they are preferably made in addition to one or more modifications to improve polynucleotide capture. This allows pores formed from the mutant monomer to effectively capture a polynucleotide and then characterise the polynucleotide, such as estimate its sequence, as discussed below.

Modifications of protein nanopores that alter their ability to interact with a polynucleotide, in particular improve their ability to capture and/or recognise or discriminate polynucleotides, are well documented in the art. For instance, such modifications are disclosed in WO 2010/034018 and WO 2010/055307. Similar modifications can be made to the lysenin monomer in accordance with this invention. Preferred modifications are disclosed International Application No. PCT/GB2013/050667 (published as WO2013153359).

Any number of modifications may be made, such as 1, 2, 5, 10, 15, 20, 30 or more modifications. Any modification(s) can be made as long as the ability of the monomer to interact with a polynucleotide is altered. Suitable modifications include, but are not limited to, amino acid substitutions, amino acid additions and amino acid deletions. The one or more modifications are preferably one or more substitutions. This is discussed in more detail below.

The one or more modifications preferably (a) alter the steric effect of the monomer, or preferably alter the steric effect of the region, (b) alter the net charge of the monomer, or preferably alter the net charge of the region, (c) alter the ability of the monomer, or preferably of the region, to hydrogen bond with the polynucleotide, (d) introduce or remove chemical groups that interact through delocalized electron pi systems and/or (e) alter the structure of the monomer, or preferably alter the structure of the region. The one or more modifications more preferably result in any combination of (a) to (e), such as (a) and (b); (a) and (c); (a) and (d); (a) and (e); (b) and (c); (b) and (d); (b) and (e); (c) and (d); (c) and (e); (d) and (e), (a), (b) and (c); (a), (b) and (d); (a), (b) and (e); (a), (c) and (d); (a), (c) and (e); (a), (d) and (e); (b), (c) and (d); (b), (c) and (e); (b), (d) and (e); (c), (d) and (e); (a), (b), (c) and d); (a), (b), (c) and (e); (a), (b), (d) and (e); (a), (c), (d) and (e); (b), (c), (d) and (e); and (a), (b), (c) and (d).

A variant of SEQ ID NO: 31 is a protein that has an amino acid sequence which varies from that of SEQ ID NO: 31 and which retains its pore forming ability. The ability of a variant to form a pore can be assayed using any method known in the art and described above.

Over the entire length of the amino acid sequence of SEQ ID NO: 31, a variant will preferably be at least 50% homologous to that sequence based on amino acid similarity or identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid similarity or identity to the amino acid sequence of SEQ ID NO: 31 over the entire sequence. There may be at least 80%, for example at least 85%, 90% or 95%, amino acid similarity or identity over a stretch of 200 or more, for example 230, 250, 270 or 280 or more, contiguous amino acids (“hard homology”). Homology can be determined as discussed below.

The hetero-oligomeric pore may also be derived from or based on CsgG. The wild type CsgG pore is formed of at least 7, at least 8, at least 9 or at least 10, preferably nine, identical monomers or subunits (i.e. it is heptameric). The sequence of one monomer or subunit of CsgG is shown in SEQ ID NO: 32. The hetero-oligomeric pore may be derived from a transmembrane protein pore comprising at least 7, at least 8, at least 9 or at least 10, preferably nine, monomers each comprising the sequence shown in SEQ ID NO: 32 or a variant thereof. The hetero-oligomeric pore produced in accordance with the invention may comprise two different monomers each the sequence shown in SEQ ID NO: 32 or a variant thereof. The first different monomer preferably comprises the sequence shown in SEQ ID NO: 32 or a variant thereof. The second different monomer preferably comprises the sequence shown in SEQ ID NO: 32 or a variant thereof. Most preferably, the first and second different monomers comprise different variants of the sequence shown in SEQ ID NO: 32.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine(S), threonine (T), tryptophan (W), tyrosine (Y) and valine (V). Standard substitution notation is also used, i.e. Q42R means that Q at position 42 is replaced with R.

The variant of SEQ ID NO: 32 comprises one or more of the following (i) one or more mutations at the following positions (i.e. mutations at one or more of the following positions) N40, D43, E44, S54, S57, Q62, R97, E101, E124, E131, R142, T150 and R192, such as one or more mutations at the following positions (i.e. mutations at one or more of the following positions) N40, D43, E44, S54, S57, Q62, E101, E131 and T150 or N40, D43, E44, E101 and E131; (ii) mutations at Y51/N55, Y51/F56, N55/F56 or Y51/N55/F56; (iii) Q42R or Q42K; (iv) K49R; (v) N102R, N102F, N102Y or N102W; (vi) D149N, D149Q or D149R; (vii) E185N, E185Q or E185R; (viii) D195N, D195Q or D195R; (ix) E201N, E201Q or E201R; (x) E203N, E203Q or E203R; and (xi) deletion of one or more of the following positions F48, K49, P50, Y51, P52, A53, S54, N55, F56 and S57. The variant may comprise any combination of (i) to (xi).

If the variant comprises any one of (i) and (iii) to (xi), it may further comprise a mutation at one or more of Y51, N55 and F56, such as at Y51, N55, F56, Y51/N55, Y51/F56, N55/F56 or Y51/N55/F56.

In (i), the variant may comprises mutations at any number and combination of N40, D43, E44, S54, S57, Q62, R97, E101, E124, E131, R142, T150 and R192. In (i), the variant preferably comprises one or more mutations at the following positions (i.e. mutations at one or more of the following positions) N40, D43, E44, S54, S57, Q62, E101, E131 and T150. In (i), the variant preferably comprises one or more mutations at the following positions (i.e. mutations at one or more of the following positions) N40, D43, E44, E101 and E131. In (i), the variant preferably comprises a mutation at S54 and/or S57. In (i), the variant more preferably comprises a mutation at (a) S54 and/or S57 and (b) one or more of Y51, N55 and F56, such as at Y51, N55, F56, Y51/N55, Y51/F56, N55/F56 or Y51/N55/F56. If S54 and/or S57 are deleted in (xi), it/they cannot be mutated in (i) and vice versa. In (i), the variant preferably comprises a mutation at T150, such as T150I. Alternatively the variant preferably comprises a mutation at (a) T150 and (b) one or more of Y51, N55 and F56, such as at Y51, N55, F56, Y51/N55, Y51/F56, N55/F56 or Y51/N55/F56. In (i), the variant preferably comprises a mutation at Q62, such as Q62R or Q62K. Alternatively the variant preferably comprises a mutation at (a) Q62 and (b) one or more of Y51, N55 and F56, such as at Y51, N55, F56, Y51/N55, Y51/F56, N55/F56 or Y51/N55/F56. The variant may comprise a mutation at D43, E44, Q62 or any combination thereof, such as D43, E44, Q62, D43/E44, D43/Q62, E44/Q62 or D43/E44/Q62. Alternatively the variant preferably comprises a mutation at (a) D43, E44, Q62, D43/E44, D43/Q62, E44/Q62 or D43/E44/Q62 and (b) one or more of Y51, N55 and F56, such as at Y51, N55, F56, Y51/N55, Y51/F56, N55/F56 or Y51/N55/F56.

In (ii) and elsewhere in this application, the/symbol means “and” such that Y51/N55 is Y51 and N55. In (ii), the variant preferably comprises mutations at Y51/N55. It has been proposed that the constriction in CsgG is composed of three stacked concentric rings formed by the side chains of residues Y51, N55 and F56 (Goyal et al, 2014, Nature, 516, 250-253). Mutation of these residues in (ii) may therefore decrease the number of nucleotides contributing to the current as the polynucleotide moves through the pore and thereby make it easier to identify a direct relationship between the observed current (as the polynucleotide moves through the pore) and the polynucleotide. Y56 may be mutated in any of the ways discussed below with reference to variants and pores useful in the method of the invention.

In (v), the variant may comprise N102R, N102F, N102Y or N102W. The variant preferably comprises (a) N102R, N102F, N102Y or N102W and (b) a mutation at one or more of Y51, N55 and F56, such as at Y51, N55, F56, Y51/N55, Y51/F56, N55/F56 or Y51/N55/F56.

In (xi), any number and combination of K49, P50, Y51, P52, A53, S54, N55, F56 and S57 may be deleted. Preferably one or more of K49, P50, Y51, P52, A53, S54, N55 and S57 may be deleted. If any of Y51, N55 and F56 are deleted in (xi), it/they cannot be mutated in (ii) and vice versa.

In (i), the variant preferably comprises one of more of the following substitutions N40R, N40K, D43N, D43Q, D43R, D43K, E44N, E44Q, E44R, E44K, S54P, S57P, Q62R, Q62K, R97N, R97G, R97L, E101N, E101Q, E101R, E101K, E101F, E101Y, E101W, E124N, E124Q, E124R, E124K, E124F, E124Y, E124W, E131D, R142E, R142N, T150I, R192E and R192N, such as one or more of N40R, N40K, D43N, D43Q, D43R, D43K, E44N, E44Q, E44R, E44K, S54P, S57P, Q62R, Q62K, E101N, E101Q, E101R, E101K, E101F, E101Y, E101W, E131D and T150I, or one or more of N40R, N40K, D43N, D43Q, D43R, D43K, E44N, E44Q, E44R, E44K, E101N, E101Q, E101R, E101K, E101F, E101Y, E101W and E131D. The variant may comprise any number and combination of these substitutions. In (i), the variant preferably comprises S54P and/or S57P. In (i), the variant preferably comprises (a) S54P and/or S57P and (b) a mutation at one or more of Y51, N55 and F56, such as at Y51, N55, F56, Y51/N55, Y51/F56, N55/F56 or Y51/N55/F56. The mutations at one or more of Y51, N55 and F56 may be any of those discussed below. In (i), the variant preferably comprises F56A/S57P or S54P/F56A. The variant preferably comprises T150I. Alternatively the variant preferably comprises a mutation at (a) T150I and (b) one or more of Y51, N55 and F56, such as at Y51, N55, F56, Y51/N55, Y51/F56, N55/F56 or Y51/N55/F56.

In (i), the variant preferably comprises Q62R or Q62K. Alternatively the variant preferably comprises (a) Q62R or Q62K and (b) a mutation at one or more of Y51, N55 and F56, such as at Y51, N55, F56, Y51/N55, Y51/F56, N55/F56 or Y51/N55/F56. The variant may comprise D43N, E44N, Q62R or Q62K or any combination thereof, such as D43N, E44N, Q62R, Q62K, D43N/E44N, D43N/Q62R, D43N/Q62K, E44N/Q62R, E44N/Q62K, D43N/E44N/Q62R or D43N/E44N/Q62K. Alternatively the variant preferably comprises (a) D43N, E44N, Q62R, Q62K, D43N/E44N, D43N/Q62R, D43N/Q62K, E44N/Q62R, E44N/Q62K, D43N/E44N/Q62R or D43N/E44N/Q62K and (b) a mutation at one or more of Y51, N55 and F56, such as at Y51, N55, F56, Y51/N55, Y51/F56, N55/F56 or Y51/N55/F56.

In (i), the variant preferably comprises D43N.

In (i), the variant preferably comprises E101R, E101S, E101F or E101N.

In (i), the variant preferably comprises E124N, E124Q, E124R, E124K, E124F, E124Y, E124W or E124D, such as E124N.

In (i), the variant preferably comprises R142E and R142N.

In (i), the variant preferably comprises R97N, R97G or R97L.

In (i), the variant preferably comprises R192E and R192N.

In (ii), the variant preferably comprises F56N/N55Q, F56N/N55R, F56N/N55K, F56N/N55S, F56N/N55G, F56N/N55A, F56N/N55T, F56Q/N55Q, F56Q/N55R, F56Q/N55K, F56Q/N55S, F56Q/N55G, F56Q/N55A, F56Q/N55T, F56R/N55Q, F56R/N55R, F56R/N55K, F56R/N55S, F56R/N55G, F56R/N55A, F56R/N55T, F56S/N55Q, F56S/N55R, F56S/N55K, F56S/N55S, F56S/N55G, F56S/N55A, F56S/N55T, F56G/N55Q, F56G/N55R, F56G/N55K, F56G/N55S, F56G/N55G, F56G/N55A, F56G/N55T, F56A/N55Q, F56A/N55R, F56A/N55K, F56A/N55S, F56A/N55G, F56A/N55A, F56A/N55T, F56K/N55Q, F56K/N55R, F56K/N55K, F56K/N55S, F56K/N55G, F56K/N55A, F56K/N55T, F56N/Y51L, F56N/Y51V, F56N/Y51A, F56N/Y51N, F56N/Y51Q, F56N/Y51S, F56N/Y51G, F56Q/Y51L, F56Q/Y51V, F56Q/Y51A, F56Q/Y51N, F56Q/Y51Q, F56Q/Y51S, F56Q/Y51G, F56R/Y51L, F56R/Y51V, F56R/Y51A, F56R/Y51N, F56R/Y51Q, F56R/Y51S, F56R/Y51G, F56S/Y51L, F56S/Y51V, F56S/Y51A, F56S/Y51N, F56S/Y51Q, F56S/Y51S, F56S/Y51G, F56G/Y51L, F56G/Y51V, F56G/Y51A, F56G/Y51N, F56G/Y51Q, F56G/Y51S, F56G/Y51G, F56A/Y51L, F56A/Y51V, F56A/Y51A, F56A/Y51N, F56A/Y51Q, F56A/Y51S, F56A/Y51G, F56K/Y51L, F56K/Y51V, F56K/Y51A, F56K/Y51N, F56K/Y51Q, F56K/Y51S, F56K/Y51G, N55Q/Y51L, N55Q/Y51V, N55Q/Y51A, N55Q/Y51N, N55Q/Y51Q, N55Q/Y51S, N55Q/Y51G, N55R/Y51L, N55R/Y51V, N55R/Y51A, N55R/Y51N, N55R/Y51Q, N55R/Y51S, N55R/Y51G, N55K/Y51L, N55K/Y51V, N55K/Y51A, N55K/Y51N, N55K/Y51Q, N55K/Y51S, N55K/Y51G, N55S/Y51L, N55S/Y51V, N55S/Y51A, N55S/Y51N, N55S/Y51Q, N55S/Y51S, N55S/Y51G, N55G/Y51L, N55G/Y51V, N55G/Y51A, N55G/Y51N, N55G/Y51Q, N55G/Y51S, N55G/Y51G, N55A/Y51L, N55A/Y51V, N55A/Y51A, N55A/Y51N, N55A/Y51Q, N55A/Y51S, N55A/Y51G, N55T/Y51L, N55T/Y51V, N55T/Y51A, N55T/Y51N, N55T/Y51Q, N55T/Y51S, N55T/Y51G, F56N/N55Q/Y51L, F56N/N55Q/Y51V, F56N/N55Q/Y51A, F56N/N55Q/Y51N, F56N/N55Q/Y51Q, F56N/N55Q/Y51S, F56N/N55Q/Y51G, F56N/N55R/Y51L, F56N/N55R/Y51V, F56N/N55R/Y51A, F56N/N55R/Y51N, F56N/N55R/Y51Q, F56N/N55R/Y51S, F56N/N55R/Y51G, F56N/N55K/Y51L, F56N/N55K/Y51V, F56N/N55K/Y51A, F56N/N55K/Y51N, F56N/N55K/Y51Q, F56N/N55K/Y51S, F56N/N55K/Y51G, F56N/N55S/Y51L, F56N/N55S/Y51V, F56N/N55S/Y51A, F56N/N55S/Y51N, F56N/N55S/Y51Q, F56N/N55S/Y51S, F56N/N55S/Y51G, F56N/N55G/Y51L, F56N/N55G/Y51V, F56N/N55G/Y51A, F56N/N55G/Y51N, F56N/N55G/Y51Q, F56N/N55G/Y51S, F56N/N55G/Y51G, F56N/N55A/Y51L, F56N/N55A/Y51V, F56N/N55A/Y51A, F56N/N55A/Y51N, F56N/N55A/Y51Q, F56N/N55A/Y51S, F56N/N55A/Y51G, F56N/N55T/Y51L, F56N/N55T/Y51V, F56N/N55T/Y51A, F56N/N55T/Y51N, F56N/N55T/Y51Q, F56N/N55T/Y51S, F56N/N55T/Y51G, F56Q/N55Q/Y51L, F56Q/N55Q/Y51V, F56Q/N55Q/Y51A, F56Q/N55Q/Y51N, F56Q/N55Q/Y51Q, F56Q/N55Q/Y51S, F56Q/N55Q/Y51G, F56Q/N55R/Y51L, F56Q/N55R/Y51V, F56Q/N55R/Y51A, F56Q/N55R/Y51N, F56Q/N55R/Y51Q, F56Q/N55R/Y51S, F56Q/N55R/Y51G, F56Q/N55K/Y51L, F56Q/N55K/Y51V, F56Q/N55K/Y51A, F56Q/N55K/Y51N, F56Q/N55K/Y51Q, F56Q/N55K/Y51S, F56Q/N55K/Y51G, F56Q/N55S/Y51L, F56Q/N55S/Y51V, F56Q/N55S/Y51A, F56Q/N55S/Y51N, F56Q/N55S/Y51Q, F56Q/N55S/Y51S, F56Q/N55S/Y51G, F56Q/N55G/Y51L, F56Q/N55G/Y51V, F56Q/N55G/Y51A, F56Q/N55G/Y51N, F56Q/N55G/Y51Q, F56Q/N55G/Y51S, F56Q/N55G/Y51G, F56Q/N55A/Y51L, F56Q/N55A/Y51V, F56Q/N55A/Y51A, F56Q/N55A/Y51N, F56Q/N55A/Y51Q, F56Q/N55A/Y51S, F56Q/N55A/Y51G, F56Q/N55T/Y51L, F56Q/N55T/Y51V, F56Q/N55T/Y51A, F56Q/N55T/Y51N, F56Q/N55T/Y51Q, F56Q/N55T/Y51S, F56Q/N55T/Y51G, F56R/N55Q/Y51L, F56R/N55Q/Y51V, F56R/N55Q/Y51A, F56R/N55Q/Y51N, F56R/N55Q/Y51Q, F56R/N55Q/Y51S, F56R/N55Q/Y51G, F56R/N55R/Y51L, F56R/N55R/Y51V, F56R/N55R/Y51A, F56R/N55R/Y51N, F56R/N55R/Y51Q, F56R/N55R/Y51S, F56R/N55R/Y51G, F56R/N55K/Y51L, F56R/N55K/Y51V, F56R/N55K/Y51A, F56R/N55K/Y51N, F56R/N55K/Y51Q, F56R/N55K/Y51S, F56R/N55K/Y51G, F56R/N55S/Y51L, F56R/N55S/Y51V, F56R/N55S/Y51A, F56R/N55S/Y51N, F56R/N55S/Y51Q, F56R/N55S/Y51S, F56R/N55S/Y51G, F56R/N55G/Y51L, F56R/N55G/Y51V, F56R/N55G/Y51A, F56R/N55G/Y51N, F56R/N55G/Y51Q, F56R/N55G/Y51S, F56R/N55G/Y51G, F56R/N55A/Y51L, F56R/N55A/Y51V, F56R/N55A/Y51A, F56R/N55A/Y51N, F56R/N55A/Y51Q, F56R/N55A/Y51S, F56R/N55A/Y51G, F56R/N55T/Y51L, F56R/N55T/Y51V, F56R/N55T/Y51A, F56R/N55T/Y51N, F56R/N55T/Y51Q, F56R/N55T/Y51S, F56R/N55T/Y51G, F56S/N55Q/Y51L, F56S/N55Q/Y51V, F56S/N55Q/Y51A, F56S/N55Q/Y51N, F56S/N55Q/Y51Q, F56S/N55Q/Y51S, F56S/N55Q/Y51G, F56S/N55R/Y51L, F56S/N55R/Y51V, F56S/N55R/Y51A, F56S/N55R/Y51N, F56S/N55R/Y51Q, F56S/N55R/Y51S, F56S/N55R/Y51G, F56S/N55K/Y51L, F56S/N55K/Y51V, F56S/N55K/Y51A, F56S/N55K/Y51N, F56S/N55K/Y51Q, F56S/N55K/Y51S, F56S/N55K/Y51G, F56S/N55S/Y51L, F56S/N55S/Y51V, F56S/N55S/Y51A, F56S/N55S/Y51N, F56S/N55S/Y51Q, F56S/N55S/Y51S, F56S/N55S/Y51G, F56S/N55G/Y51L, F56S/N55G/Y51V, F56S/N55G/Y51A, F56S/N55G/Y51N, F56S/N55G/Y51Q, F56S/N55G/Y51S, F56S/N55G/Y51G, F56S/N55A/Y51L, F56S/N55A/Y51V, F56S/N55A/Y51A, F56S/N55A/Y51N, F56S/N55A/Y51Q, F56S/N55A/Y51S, F56S/N55A/Y51G, F56S/N55T/Y51L, F56S/N55T/Y51V, F56S/N55T/Y51A, F56S/N55T/Y51N, F56S/N55T/Y51Q, F56S/N55T/Y51S, F56S/N55T/Y51G, F56G/N55Q/Y51L, F56G/N55Q/Y51V, F56G/N55Q/Y51A, F56G/N55Q/Y51N, F56G/N55Q/Y51Q, F56G/N55Q/Y51S, F56G/N55Q/Y51G, F56G/N55R/Y51L, F56G/N55R/Y51V, F56G/N55R/Y51A, F56G/N55R/Y51N, F56G/N55R/Y51Q, F56G/N55R/Y51S, F56G/N55R/Y51G, F56G/N55K/Y51L, F56G/N55K/Y51V, F56G/N55K/Y51A, F56G/N55K/Y51N, F56G/N55K/Y51Q, F56G/N55K/Y51S, F56G/N55K/Y51G, F56G/N55S/Y51L, F56G/N55S/Y51V, F56G/N55S/Y51A, F56G/N55S/Y51N, F56G/N55S/Y51Q, F56G/N55S/Y51S, F56G/N55S/Y51G, F56G/N55G/Y51L, F56G/N55G/Y51V, F56G/N55G/Y51A, F56G/N55G/Y51N, F56G/N55G/Y51Q, F56G/N55G/Y51S, F56G/N55G/Y51G, F56G/N55A/Y51L, F56G/N55A/Y51V, F56G/N55A/Y51A, F56G/N55A/Y51N, F56G/N55A/Y51Q, F56G/N55A/Y51S, F56G/N55A/Y51G, F56G/N55T/Y51L, F56G/N55T/Y51V, F56G/N55T/Y51A, F56G/N55T/Y51N, F56G/N55T/Y51Q, F56G/N55T/Y51S, F56G/N55T/Y51G, F56A/N55Q/Y51L, F56A/N55Q/Y51V, F56A/N55Q/Y51A, F56A/N55Q/Y51N, F56A/N55Q/Y51Q, F56A/N55Q/Y51S, F56A/N55Q/Y51G, F56A/N55R/Y51L, F56A/N55R/Y51V, F56A/N55R/Y51A, F56A/N55R/Y51N, F56A/N55R/Y51Q, F56A/N55R/Y51S, F56A/N55R/Y51G, F56A/N55K/Y51L, F56A/N55K/Y51V, F56A/N55K/Y51A, F56A/N55K/Y51N, F56A/N55K/Y51Q, F56A/N55K/Y51S, F56A/N55K/Y51G, F56A/N55S/Y51L, F56A/N55S/Y51V, F56A/N55S/Y51A, F56A/N55S/Y51N, F56A/N55S/Y51Q, F56A/N55S/Y51S, F56A/N55S/Y51G, F56A/N55G/Y51L, F56A/N55G/Y51V, F56A/N55G/Y51A, F56A/N55G/Y51N, F56A/N55G/Y51Q, F56A/N55G/Y51S, F56A/N55G/Y51G, F56A/N55A/Y51L, F56A/N55A/Y51V, F56A/N55A/Y51A, F56A/N55A/Y51N, F56A/N55A/Y51Q, F56A/N55A/Y51S, F56A/N55A/Y51G, F56A/N55T/Y51L, F56A/N55T/Y51V, F56A/N55T/Y51A, F56A/N55T/Y51N, F56A/N55T/Y51Q, F56A/N55T/Y51S, F56A/N55T/Y51G, F56K/N55Q/Y51L, F56K/N55Q/Y51V, F56K/N55Q/Y51A, F56K/N55Q/Y51N, F56K/N55Q/Y51Q, F56K/N55Q/Y51S, F56K/N55Q/Y51G, F56K/N55R/Y51L, F56K/N55R/Y51V, F56K/N55R/Y51A, F56K/N55R/Y51N, F56K/N55R/Y51Q, F56K/N55R/Y51S, F56K/N55R/Y51G, F56K/N55K/Y51L, F56K/N55K/Y51V, F56K/N55K/Y51A, F56K/N55K/Y51N, F56K/N55K/Y51Q, F56K/N55K/Y51S, F56K/N55K/Y51G, F56K/N55S/Y51L, F56K/N55S/Y51V, F56K/N55S/Y51A, F56K/N55S/Y51N, F56K/N55S/Y51Q, F56K/N55S/Y51S, F56K/N55S/Y51G, F56K/N55G/Y51L, F56K/N55G/Y51V, F56K/N55G/Y51A, F56K/N55G/Y51N, F56K/N55G/Y51Q, F56K/N55G/Y51S, F56K/N55G/Y51G, F56K/N55A/Y51L, F56K/N55A/Y51V, F56K/N55A/Y51A, F56K/N55A/Y51N, F56K/N55A/Y51Q, F56K/N55A/Y51S, F56K/N55A/Y51G, F56K/N55T/Y51L, F56K/N55T/Y51V, F56K/N55T/Y51A, F56K/N55T/Y51N, F56K/N55T/Y51Q, F56K/N55T/Y51S, F56K/N55T/Y51G, F56E/N55R, F56E/N55K, F56D/N55R, F56D/N55K, F56R/N55E, F56R/N55D, F56K/N55E or F56K/N55D.

In (ii), the variant preferably comprises Y51R/F56Q, Y51N/F56N, Y51M/F56Q, Y51L/F56Q, Y51I/F56Q, Y51V/F56Q, Y51A/F56Q, Y51P/F56Q, Y51G/F56Q, Y51C/F56Q, Y51Q/F56Q, Y51N/F56Q, Y51S/F56Q, Y51E/F56Q, Y51D/F56Q, Y51K/F56Q or Y51H/F56Q.

In (ii), the variant preferably comprises Y51T/F56Q, Y51Q/F56Q or Y51A/F56Q.

In (ii), the variant preferably comprises Y51T/F56F, Y51T/F56M, Y51T/F56L, Y51T/F56I, Y51T/F56V, Y51T/F56A, Y51T/F56P, Y51T/F56G, Y51T/F56C, Y51T/F56Q, Y51T/F56N, Y51T/F56T, Y51T/F56S, Y51T/F56E, Y51T/F56D, Y51T/F56K, Y51T/F56H or Y51T/F56R.

In (ii), the variant preferably comprises Y51T/N55Q, Y51T/N55S or Y51T/N55A.

In (ii), the variant preferably comprises Y51A/F56F, Y51A/F56L, Y51A/F56I, Y51A/F56V, Y51A/F56A, Y51A/F56P, Y51A/F56G, Y51A/F56C, Y51A/F56Q, Y51A/F56N, Y51A/F56T, Y51A/F56S, Y51A/F56E, Y51A/F56D, Y51A/F56K, Y51A/F56H or Y51A/F56R.

In (ii), the variant preferably comprises Y51C/F56A, Y51E/F56A, Y51D/F56A, Y51K/F56A, Y51H/F56A, Y51Q/F56A, Y51N/F56A, Y51S/F56A, Y51P/F56A or Y51V/F56A.

In (xi), the variant preferably comprises deletion of Y51/P52, Y51/P52/A53, P50 to P52, P50 to A53, K49 to Y51, K49 to A53 and replacement with a single proline (P), K49 to S54 and replacement with a single P, Y51 to A53, Y51 to S54, N55/F56, N55 to S57, N55/F56 and replacement with a single P, N55/F56 and replacement with a single glycine (G), N55/F56 and replacement with a single alanine (A), N55/F56 and replacement with a single P and Y51N, N55/F56 and replacement with a single P and Y51Q, N55/F56 and replacement with a single P and Y51S, N55/F56 and replacement with a single G and Y51N, N55/F56 and replacement with a single G and Y51Q, N55/F56 and replacement with a single G and Y51S, N55/F56 and replacement with a single A and Y51N, N55/F56 and replacement with a single A/Y51Q or

N55/F56 and replacement with a single A and Y51S.

Preferred variants of SEQ ID NO: 32 are disclosed in International Application No. PCT/EP2015/069965.

A variant of SEQ ID NO: 32 is a protein that has an amino acid sequence which varies from that of SEQ ID NO: 32 and which retains its pore forming ability. The ability of a variant to form a pore can be assayed using any method known in the art and described above.

Over the entire length of the amino acid sequence of SEQ ID NO: 32, a variant will preferably be at least 50% homologous to that sequence based on amino acid similarity or identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid similarity or identity to the amino acid sequence of SEQ ID NO: 32 over the entire sequence. There may be at least 80%, for example at least 85%, 90% or 95%, amino acid similarity or identity over a stretch of 200 or more, for example 230, 250, 270 or 280 or more, contiguous amino acids (“hard homology”). Homology can be determined as discussed below.

Amino acid substitutions may be made to the amino acid sequence of SEQ ID NO: 4, 31 or 32 in addition to those discussed above, for example up to 1, 2, 3, 4, 5, 10, 20 or 30 substitutions. Conservative substitutions may be made as discussed above.

One or more amino acid residues of the amino acid sequence of SEQ ID NO: 4, 31 or 32 may additionally be deleted from the polypeptides described above. Up to 1, 2, 3, 4, 5, 10, 20 or 30 residues may be deleted, or more.

Variants may be fragments of SEQ ID NO: 4, 31 or 32. Such fragments retain pore-forming activity. Fragments may be at least 50, 100, 200 or 250 amino acids in length. A fragment preferably comprises the pore-forming domain of SEQ ID NO: 4, 31 or 32. Fragments typically include residues 119, 121, 135. 113 and 139 of SEQ ID NO: 4.

One or more amino acids may be alternatively or additionally added to the polypeptides described above. An extension may be provided at the amino terminus or carboxy terminus of the amino acid sequence of SEQ ID NO: 4, 31 or 32 or a variant or fragment thereof. The extension may be quite short, for example from 1 to 10 amino acids in length. Alternatively, the extension may be longer, for example up to 50 or 100 amino acids. A carrier protein may be fused to a pore or variant.

As discussed above, a variant of SEQ ID NO: 4, 31 or 32 is a subunit that has an amino acid sequence which varies from that of SEQ ID NO: 4, 31 or 32 and which retains its ability to form a pore. A variant typically contains the regions of SEQ ID NO: 4, 31 or 32 that are responsible for pore formation. The pore forming ability of α-HL, which contains a β-barrel, is provided by β-strands in each subunit. A variant of SEQ ID NO: 4 typically comprises the regions in SEQ ID NO: 4 that form β-strands. The amino acids of SEQ ID NO: 4 that form β-strands are discussed above. One or more modifications can be made to the regions of SEQ ID NO: 4 that form β-strands as long as the resulting variant retains its ability to form a pore.

Specific modifications that can be made to the β-strand regions of SEQ ID NO: 4 are discussed above.

A variant of SEQ ID NO: 4, 31 or 32 preferably includes one or more modifications, such as substitutions, additions or deletions, within its α-helices and/or loop regions. Amino acids that form α-helices and loops are discussed above.

The variant of SEQ ID NO: 4, 31 or 32 may be modified to affects its oligomerisation and/or to assist its identification or purification as discussed below.

In some embodiments, the hetero-oligomeric pore is chemically modified. The pore can be chemically modified in any way and at any site. Suitable modifications are discussed below. Such modifications can be applied to any of the pores produced in the invention.

### Constructs

The hetero-oligomeric pore of the invention may comprise a construct comprising two or more covalently attached monomers. The first different monomer may form part of a first different construct comprising two or more genetically fused monomers. The second different monomer may form part of a second different construct comprising two or more genetically fused monomers. The first and second different constructs may differ from one another in any of the ways discussed above and below.

The first different construct and second different construct retain their ability to form a pore. This may be determined as discussed above. Each construct may comprise at least 2, at least 3, at least 4, at least 5, at least 6, at least 7, at least 8, at least 9 or at least 10 monomers. Each construct preferably comprises two monomers. The two or more monomers may be the same or different.

The monomers in the construct are preferably approximately the same length or are the same length. The barrels of the monomers in the construct are preferably approximately the same length or are the same length. Length may be measured in number of amino acids and/or units of length. The monomers in the construct preferably have the same number of amino acids deleted from the barrel, such as from positions 72 to 82 and/or positions 111 to 121 in MspA (SEQ ID NO: 2). As discussed below, one or more of the different constructs may be attached to a tag or BasTL sequence or fragment thereof which makes it longer than the other constructs in the pore.

The monomers in the construct are genetically fused. Monomers are genetically fused if the whole construct is expressed from a single polynucleotide sequence. The coding sequences of the monomers may be combined in any way to form a single polynucleotide sequence encoding the construct.

The monomers may be genetically fused in any configuration. The monomers may be fused via their terminal amino acids. For instance, the amino terminus of the one monomer may be fused to the carboxy terminus of another monomer. The second and subsequent monomers in the construct (in the amino to carboxy direction) may comprise a methionine at their amino terminal ends (each of which is fused to the carboxy terminus of the previous monomer). For instance, if M is a monomer (without an amino terminal methionine) and mM is a monomer with an amino terminal methionine, the construct may comprise the sequence mM-mM, mM-mM-mM or mM-mM-mM-mM. The presences of these methionines typically results from the expression of the start codons (i.e. ATGs) at the 5′ end of the polynucleotides encoding the monomers within the polynucleotide encoding the entire construct. The second and subsequent monomer in the construct (in the amino to carboxy direction) may lack a methionine (e.g. mM-M, mM-M-M or mM-M-M-M).

The two or more monomers may be genetically fused directly together. The monomers are preferably genetically fused using a linker. The linker may be designed to constrain the mobility of the monomers. Preferred linkers are amino acid sequences (i.e. peptide linkers). Any of the peptide linkers discussed below may be used.

The pore contains sufficient constructs and, if necessary, monomers to form the pore. For instance, an octameric pore may comprise (a) four constructs each comprising two monomers, (b) two constructs each comprising four monomers or (b) one construct comprising two monomers and six monomers that do not form part of a construct. For instance, a nonameric pore may comprise (a) four constructs each comprising two monomers and one monomer that does not form part of a construct, (b) two constructs each comprising four monomers and a monomer that does not form part of a construct or (b) one construct comprising two monomers and seven monomers that do not form part of a construct. Other combinations of constructs and monomers can be envisaged by the skilled person.

A pore of the invention typically contains (a) one construct comprising two monomers and (b) 5, 6, 7 or 8 monomers. The construct may be any of those discussed above.

Another typical pore comprises more than one construct of the invention, such as two, three or four constructs of the invention. If necessary, such pores further comprise sufficient additional monomers or constructs to form the pore. The additional monomer(s) may be any of those discussed above.

A further pore of the invention comprises only constructs comprising 2 monomers, for example a pore may comprise 4, 5, 6, 7 or 8 constructs comprising 2 monomers.

One or more of the constructs may be chemically-modified as discussed above. In all of the discussion below, embodiments which relate to first different monomer and/or the second different monomer, especially the specific stoichiometric ratios, equally apply to the first different construct and/or and second different construct. The term monomer and construct are interchangeable in all of the discussion below.

### Specific Stoichiometric Ratio

The hetero-oligomeric pore produced by the method of the invention comprises two different monomers in a specific stoichiometric ratio. The specific stoichiometric ratio is typically pre-determined. It may also be called the desired stoichiometric ratio.

The specific stoichiometric ratio is the ratio of the first different monomer to the second different monomer in the hetero-oligomeric pore. If the hetero-oligomeric pore comprises 3 monomers, the specific stoichiometric ratio of the first different monomer to the second different monomer may be 1:2 or 2:1. If the hetero-oligomeric pore comprises 4 monomers, the specific stoichiometric ratio of the first different monomer to the second different monomer may be 1:3, 2:2 or 3:1. If the hetero-oligomeric pore comprises 5 monomers, the specific stoichiometric ratio of the first different monomer to the second different monomer may be 1:4, 2:3, 3:2 or 4:1. If the hetero-oligomeric pore comprises 6 monomers, the specific stoichiometric ratio of the first different monomer to the second different monomer may be 1:5, 2:4, 3:3, 4:2 or 5:1. If the hetero-oligomeric pore comprises 7 monomers, the specific stoichiometric ratio of the first different monomer to the second different monomer may be 1:6, 2:5, 3:4, 4:3, 5:2 or 6:1. If the hetero-oligomeric pore comprises 8 monomers, the specific stoichiometric ratio of the first different monomer to the second different monomer may be 1:7, 2:6, 3:5, 4:4, 5:3, 6:2 or 7:1. If the hetero-oligomeric pore comprises 9 monomers, the specific stoichiometric ratio of the first different monomer to the second different monomer may be 1:8, 2:7, 3:6, 4:5, 5:4, 6:3, 7:2 or 8:1. If the hetero-oligomeric pore comprises 10 monomers, the specific stoichiometric ratio of the first different monomer to the second different monomer may be 1:9, 2:8, 3:7, 4:6, 5:5, 6:4, 7:3, 8:2 or 9:1.

The specific stoichiometric ratio of the first different monomer to the second different monomer is at least 5:1, such as at least 6:1, at least 7:1, at least 8:1 or at least 9:1. The specific stoichiometric ratio of the first different monomer to the second different monomer is most preferably 6:1 or 7:1.

The method of the invention produces a hetero-oligomeric pore comprising two different monomers in a specific stoichiometric ratio. This means that the majority of the pores produced in the cell comprise the two different monomers in the specific stoichiometric ratio. For instance, at least 55% of the hetero-oligomeric pores produced by the cell comprise the two different monomers in the specific stoichiometric ratio. At least 60%, such at least 65%, at least 70%, at least 75%, at least 80%, at least 85%, at least 90%, at least 95%, at least 97%, at least 98% or at least 99%, of the hetero-oligomeric pores produced by the cell comprise the two different monomers in the specific stoichiometric ratio. In some instances, all, i.e. 100%, of the hetero-oligomeric pores produced by the cell comprise the two different monomers in the specific stoichiometric ratio. The % of hetero-oligomeric pores produced by the cell which comprise the two different monomers in the specific stoichiometric ratio can be measured using methods in the art. For instance, if one of the different monomers is tagged as discussed below, hetero-oligomeric pores comprising different ratios of the different monomers will have different sizes. The different sized hetero-oligomeric pores can be identified and quantified using gel electrophoresis.

### Vectors

The method of the invention comprises transfecting or transforming a cell with the two different monomers. Transfection or transformation involves the introduction of a polynucleotide, such as a nucleic acid, into the cell. Transfection or transformation typically concerns non-viral methods.

The cell is transfected or transformed with the first different monomer in a first inducible vector or first inducible expression vector. The cell is also transfected or transformed with the second different monomer in a second inducible vector or second inducible expression vector. The method may comprise transfecting or transforming the cell with the first and second inducible vectors or inducible expression vectors in any order. The method may comprise transfecting or transforming the cell with the first inducible vector or first inducible expression vector before the second inducible vector or the second inducible expression vector. The method may comprise transfecting or transforming the cell with the second inducible vector or second inducible expression vector before the first inducible vector or the first inducible expression vector. The method may comprise transfecting or transforming the cell with the first and second inducible vectors or inducible expression vectors at the same time. Before the first and second inducible vectors are induced such that the cell produces the hetero-oligomeric pore comprising the first and second different monomers in the specific stoichiometric ratio, the cell is transfected or transformed with both the first and second inducible vectors or inducible expression vectors.

The cell is typically transfected or transformed with a polynucleotide sequence encoding the first different monomer in a first inducible vector or first inducible expression vector. The cell is also typically transfected or transformed with a polynucleotide sequence encoding the second different monomer in a second inducible vector or second inducible expression vector.

Polynucleotide sequences conceding the monomers may be derived and replicated using standard methods in the art. Chromosomal DNA encoding wild-type pores may be extracted from a pore producing organism, such as Staphylococcus aureus, Mycobacterium smegmatis or Escherichia coli. The gene encoding the pore subunit may be amplified using PCR involving specific primers. The amplified sequence may then undergo site-directed mutagenesis. Suitable methods of site-directed mutagenesis are known in the art and include, for example, combined chain reaction. Polynucleotides can be made using well-known techniques, such as those described in Sambrook, J. and Russell, D. (2001). Molecular Cloning: A Laboratory Manual, 3rd Edition. Cold Spring Harbor Laboratory Press, Cold Spring Harbor, NY. They can also be obtained commercially.

The resulting polynucleotide sequence may then be incorporated into a recombinant replicable vector such as a cloning vector. The vector may be used to replicate the polynucleotide in a compatible host cell. Thus polynucleotide sequences may be made by introducing a polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into suitable expression vector. In an expression vector, the polynucleotide sequence is typically operably linked to a control sequence which is capable of providing for the expression of the coding sequence by the host cell. Such expression vectors can be used to express a monomer. The polynucleotide sequence encoding the first different monomer is preferably operably linked to a first inducible control sequence, such as a first inducible promoter, in the first inducible vector or first inducible expression vector. The polynucleotide sequence encoding the second different monomer is preferably operably linked to a second inducible control sequence, such as a second inducible promoter, in the second inducible vector or second inducible expression vector.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide sequences may be introduced into the vector.

The first inducible vector and the second inducible vector are typically inducible because they each comprise an inducible promoter. The first inducible vector typically comprises a first inducible promoter and the second inducible vector typically comprises a second inducible promoter. The first and/or second inducible promoter may be an arabinose promoter, a proprionate promoter, a rhamnose-inducible promoter, a xylose promoter or a lactose promoter. The polynucleotide sequence encoding the first different monomer is preferably operably linked to an arabinose promoter, a proprionate promoter, a rhamnose-inducible promoter, a xylose promoter or a lactose promoter. The polynucleotide sequence encoding the second different monomer is preferably operably linked to an arabinose promoter, a proprionate promoter, a rhamnose-inducible promoter, a xylose promoter or a lactose promoter.

The promoters in the first inducible vector and the second inducible vector are preferably different. The first inducible vector preferably comprises a rhamnose-inducible promoter and the second inducible vector comprises a lactose promoter. The polynucleotide sequence encoding the first different monomer is preferably operably linked to a rhamnose-inducible promoter and the polynucleotide sequence encoding the second different monomer is preferably operably linked to a lactose promoter. The first inducible vector or first inducible promoter is preferably induced by rhamnose. The second inducible vector or the second inducible promoter is preferably induced by Isopropyl β-D-thiogalactopyranoside (IPTG).

The first and second inducible vectors are preferably differentially inducible. Differential inducibility is discussed in more detail below.

The first inducible vector and/or the second inducible vector may be for example, plasmid, virus or phage vectors. The first inducible vector and/or the second inducible vector may comprise an origin of replication.

The first inducible vector and/or the second inducible vector may contain one or more selection marker genes, for example a tetracycline resistance gene, triclosan resistance gene, ampicillin resistance gene or kanamycin resistance gene. The first inducible vector preferably comprises a first selection marker and the second inducible vector preferably comprises a second selection marker. The first and second selection markers are preferably different from one another. If the selection markers are different, it is straightforward to confirm that the cell has been transfected or transformed with both vectors. The first selection marker is preferably a gene providing resistance to kanamycin. The second selection marker is preferably a gene providing resistance to ampicillin.

The cell transfected or transformed in accordance with the invention may be referred to as the host cell. The method may comprise contacting the cell with the first and second inducible vectors under conditions which will transfect or transform the cell. Suitable conditions are known in the art (see, for instance, Sambrook, J. and Russell, D. supra).

The cell typically expresses the first and/or second different monomers at a high level. Cells will typically be chosen to be compatible with the expression vectors used to transfect or transform the cell. Suitable cells for use in the invention include prokaryotic cells and eukaryotic cells. The prokaryotic cell is preferably a bacterial cell. Suitable bacterial cells include, but are not limited to, Escherichia coli, Corynebacterium and Pseudomonas fluorescens. Any E. coli cell with a DE3 lysogen, for example C41 (DE3), BL21 (DE3), JM109 (DE3), B834 (DE3), TUNER, Origami and Origami B, can express a vector comprising the T7 promoter.

Suitable eukaryotic cells include, but are not limited to, Saccharomyces cerevisiae, Pichia pastoris, filamentous fungi, such as Aspergillus, Trichoderma and Myceliophthora thermophila C1, baculovirus-infected insect cells, such as Sf9, Sf21 and High Five strains, non-lytic insect cells, Leishmania cells, plant cells, such as tobacco plant cells, and mammalian cells, such as Bos primigenius cells (Bovine), Mus musculus cells (Mouse), Chinese Hamster Ovary (CHO) cells, Human Embryonic Kidney (HEK) cells, Baby Hamster Kidney (BHK) cells and HeLa cells. Other preferred mammalian cells include, but are not limited to, PC12, HEK293, HEK293A, HEK293T, CHO, BHK-21, HeLa, ARPE-19, RAW264.7 and COS cells.

The host cell is preferably Escherichia coli.

The recombinantly-expressed first and second different monomers typically self-assemble into a hetero-oligomeric pore in the cell membrane. In step (c), the first and second monomers are preferably expressed in the cell in the specific stoichiometric ratio and the hetero-oligomeric pore forms in cell membrane.

### Differential Induction

The method preferably comprises differentially inducing the first and second inducible vectors. This typically involves inducing the first and second inducible vectors in different ways. For instance, the first inducible vector may be induced by rhamnose and the second inducible vector may be induced by isopropyl β-D-thiogalactopyranoside (IPTG). A skilled person can perform control experiments to determine the effect of differential induction on the stoichiometric ratio of the first and second different monomers in the hetero-oligomeric pore.

The method preferably comprises inducing the first and second inducible vectors to differing extents. This typically involves inducing the first and second inducible vectors to differing degrees. The first and second inducible vectors are typically induced to differing extents or degrees using different concentrations of the inducing chemicals or molecules. A skilled person can perform control experiments to determine the effect of inducing the vectors to differing degrees or extents on the stoichiometric ratio of the first and second different monomers in the hetero-oligomeric pore.

The first and second inducible vectors may be differentially induced to differing extents.

### Ratio of Expression

The hetero-pore produced using the invention may comprise the first and second different monomers in the specific stoichiometric ratio because the cell expresses the first and second different monomers in a specific ratio. In step (c), the first and second different monomers are preferably expressed by the cell in a ratio that allows the formation of the hetero-oligomeric pore comprising the first and second different monomers in the specific stoichiometric ratio. However, the first and second different monomers do not need to be expressed in the specific stoichiometric ratio. The ratio in which the first and second different monomers are expressed may allow the formation of different hetero-oligomeric pores comprising different ratios of the first and second different monomers including the specific stoichiometric ratio. For instance, if the hetero-oligomeric pore comprises 8 monomers and the specific stoichiometric ratio of the first different monomer to the second different monomer is 7:1, the different monomers may be expressed in a ratio that allows the formation of the hetero-oligomeric pores comprising the first and second different monomers in ratio 6:2, 7:1 and 8:0. The hetero-oligomeric pore having the specific stoichiometric ratio may be purified as discussed in more detail below.

The ratio of expression of the first different monomer to the second different monomer can be measured using routine methods, such as SDS-PAGE, western blotting or mass spectroscopy. The ratio of expression can be affected by differentially inducing the first and second inducible vectors and/or inducing them to different extents as discussed above.

The ratio of expression of the first and second different monomers may also be affected by modifying one or both of them. At least one of, such as both of, the first and second different monomers is preferably modified to affect its expression compared with its expression in the absence of the modification. The first different monomer may be modified to increase or decrease its expression compared with its expression in the absence of the modification. The second different monomer may be modified to increase or decrease its expression compared with its expression in the absence of the modification. Preferred combinations of modification or lack thereof are shown in Table 1 below.

TABLE 1

| First different monomer | Second different monomer |
|---|---|
| Modification which increases | No modification |
| its expression | |
| No modification | Modification which increases |
| | its expression |
| Modification which decreases | No modification |
| its expression | |
| No modification | Modification which decreases |
| | its expression |
| Modification which increases | Modification which decreases |
| its expression | its expression |
| Modification which decreases | Modification which increases |
| its expression | its expression |
| Modification which increases | Modification which increases |
| its expression | its expression |
| Modification which decreases | Modification which decreases |
| its expression | its expression |

The expression of the first different monomer and/or second different monomer can be affected to any degree. For instance, the expression of the first different monomer and/or second different monomer can be increased or decreased by at least 5%, such as by at least 10%, at least 20%, at least 30%, at least 40%, at least 50%, at least 60%, at least 70%, at least 80%, at least 90%, at least 100%, at least 150%, at least 200%, at least 300%, at least 400%, at least 500%. The level of expression of a monomer can be determined using routine methods, such as SDS-PAGE, western blotting, UV280 concentration measurement or mass spectroscopy. The ability of a modification to affect the expression of a monomer can be determined by comparing the expression of the modified monomer with the expression of the unmodified monomer in the same cell under the same conditions.

Increasing or decreasing the expression of the two monomers to different extents can help to ensure that the monomers are expressed in approximately the specific stoichiometric ratio.

A skilled person will be able to design modifications which affect the expression of the monomer(s). The modification is preferably the genetic fusion of a peptide, polypeptide or protein sequence to the monomer such that the peptide, polypeptide or protein sequence is expressed with the monomer. The monomer and the peptide, polypeptide or protein sequence are genetically fused if they are expressed from a single polynucleotide sequence. Preferred methods of decreasing the expression of a monomer are discussed in more detail below with reference to tags.

Modification which Affects Oligomerisation

The hetero-pore produced using the invention may comprise the first and second different monomers in the specific stoichiometric ratio because one or both of the first and second different monomers are modified to affect their ability to oligomerise. At least one of, such as both of, the first and second different monomers is preferably modified to affect its ability to oligomerise with itself or the other different monomer. The first different monomer may be modified to increase or decrease its ability to oligomerise with the second different monomer. The second different monomer may be modified to increase or decrease its ability to oligomerise with the first different monomer. Increasing the ability of a monomer to oligomerise will increase its incidence in the hetero-oligomeric pores produced using the method and can therefore influence the specific stoichiometric ratio. Conversely, decreasing the ability of a monomer to oligomerise will decrease its incidence in the hetero-oligomeric pores produced using the method.

Preferred combinations of modifications or a lack thereof are shown in the Table 2 below.

TABLE 2

| First different monomer | Second different monomer |
|---|---|
| Modification which increases | No modification |
| its ability to oligomerise | |
| No modification | Modification which increases |
| | its ability to oligomerise |
| Modification which decreases | No modification |
| its ability to oligomerise | |
| No modification | Modification which decreases |
| | its ability to oligomerise |
| Modification which increases | Modification which decreases |
| its ability to oligomerise | its ability to oligomerise |
| Modification which decreases | Modification which increases |
| its ability to oligomerise | its ability to oligomerise |
| Modification which increases | Modification which increases |
| its ability to oligomerise | its ability to oligomerise |
| Modification which decreases | Modification which decreases |
| its ability to oligomerise | its ability to oligomerise |

The ability of the first different monomer and/or second different monomer to oligomerise with the other monomer can be affected to any degree. For instance, the ability of the first different monomer and/or second different monomer to oligomerise with the other monomer can be increased or decreased by at least 5%, such as by at least 10%, at least 20%, at least 30%, at least 40%, at least 50%, at least 60%, at least 70%, at least 80%, at least 90%, at least 100%, at least 150%, at least 200%, at least 300%, at least 400%, at least 500%. The ability of a monomer to oligomerise can be determined using routine methods such as SDS-PAGE, western blotting, UV280 concentration measurement, size exclusion chromatography or mass spectroscopy. The ability of a modification to affect the oligomerisation of a monomer can be determined by comparing the oligomerisation of the modified monomer with the oligomerisation of the unmodified monomer with the same monomer under the same conditions.

Increasing or decreasing the ability of the two monomers to oligomerise to different extents can help to ensure that the hetero-oligomeric pore comprises the two different monomers in the specific stoichiometric ratio.

A skilled person will be able to design modifications which affect the ability of the monomer(s) to oligomerise. The modification is preferably the genetic fusion of a peptide, polypeptide or protein sequence to the monomer such that the peptide, polypeptide or protein sequence is expressed with the monomer. Genetic fusion is defined above. Genetic fusion may be at the amino (N) and/or carboxy (C) terminus of the monomer(s). Preferred methods of decreasing the ability of a monomer to oligomerise are discussed in more detail below with reference to tags. The modification is preferably a truncation of the monomer(s). The truncation may be at the amino (N) and/or carboxy (C) terminus of the monomer(s). The modification is preferably one or more mutations of the amino acids/regions of the monomer(s) responsible for oligomerisation.

The amino acids and regions of Staphylococcal alpha-hemolysin responsible for oligomerisation are known in the art. Walker and Bayley, Journal of Biological Chemistry, Vol. 270, No. 39, Issue of September 29, pp. 23065-23071, 1995 discloses key Residues for membrane binding, oligomerisation, and pore forming activity of Staphylococcal alpha-hemolysin identified by cysteine scanning mutagenesis and targeted chemical modification. Panchal and Bayley, Journal of Biological Chemistry, Vol. 270, No. 39, Issue of September 29, pp. 23072-23076, 1995 discloses interactions between residues in Staphylococcal alpha-hemolysin revealed by reversion mutagenesis. Cheley et al., Protein Engineering vol. 10 no. 12 pp. 1433-1443, 1997 discloses spontaneous oligomerisation of a staphylococcal α-hemolysin conformationally constrained by removal of residues that form the transmembrane beta-barrel. Jayasinghe et al., The Journal Of Biological Chemistry Vol. 281, No. 4, pp. 2195-2204 Jan. 27, 2006 discloses the role of the amino latch of Staphylococcal alpha-hemolysin in pore formation and reveals a co-operative interaction between the n terminus and position 217.

### Tags

Tags may also be used to allow the first and second different monomers to be expressed in the cell in the specific stoichiometric ratio and/or to affect the ability of the monomer to oligomerise. The tag is preferably a peptide or a polypeptide. One or more peptide or polypeptide tags may be genetically fused to the first and/or second different monomer. The monomer and the tag(s) are genetically fused if they are expressed from a single polynucleotide sequence. The presence of the one or more tags may increase or decrease the ability of a different monomer to be expressed. The presence of the one or more tags may increase or decrease the ability of a different monomer to oligomerise with itself or the other monomers in the hetero-oligomeric pore.

The second different monomer is preferably genetically fused to a peptide or polypeptide tag which reduces its ability to oligomerise with itself or reduces its expression compared with its expression in the absence of the tag. The peptide or polypeptide tag is preferably genetically fused at the carboxy (C) terminus of the second different monomer and reduces its ability to oligomerise with itself. The ability of the second different monomer to oligomerise in the presence and absence of the tag can be measured as discussed above.

The peptide or polypeptide tag is preferably genetically fused at the amino (N) terminus of the second different monomer and reduces its expression compared with its expression in the absence of the tag. The expression of the second different monomer in the presence and absence of the tag can be measured as discussed above.

The peptide or polypeptide tag may be any length. The peptide is preferably 1, 2, 3, 4 or 5 amino acids in length. The polypeptide is preferably greater than 5 amino acids in length, such as 8, 10, 12, 20, 30, 40, 50 or 100 amino acids in length or more. The peptide or polypeptide may comprise any naturally-occurring or non-naturally occurring amino acids.

The tag preferably comprises two or more consecutive arginine (R) residues or aspartic acid (D) residues. The tag more preferably comprises (a) 1, 2, 3, 4, 5, 6, 7, 8, 9, 10, 11, 12, 13, 14, 15, 16, 17, 18, 19 or 20 consecutive arginine (R) residues or aspartic acid (D) residues and/or (b) 1, 2, 3, 4, 5, 6, 7, 8, 9, 10, 11, 12, 13, 14, 15, 16, 17, 18, 19 or 20 consecutive histidine (H) residues. The tag most preferably comprises (a) 4, 6, 8, or 10 consecutive arginine (R) residues or aspartic acid (D) residues and/or (b) 6 or 9 consecutive histidine (H) residues.

The tag preferably further comprises serine-glycine (SG), asparagine-glycine-aspartic acid-serine (NGDS) or glycine-aspartic acid-serine-glycine (GDSG).

Preferred tags include, but are not limited to, R8SG, D6SG, R6SG, R8, NGDSD6SG, D4SG, R4SG, D4, D6, GDSGD4SG, R4H6, D4H6, D8, R6, D10, R4, D4, D8H6, R8H9, D10H6, R6H6 and D6.

The second different monomer preferably has one of the following tags genetically fused at its amino (N) terminus: R8SG, D6SG, R6SG, R8, NGDSD6SG, D4SG, R4SG, D4, D6 and GDSGD4SG. The second different monomer preferably has one of the following tags genetically fused at its carboxy (C) terminus: R4H6, D4H6, D8, R6, D10, R4, D4, D8H6, R8, R8H9, D10H6, R6H6 and D6. The second different monomer preferably has one of the following tags genetically fused at its carboxy (C) terminus: D4H6, D8, D10, D4, D8H6, R8, R8H9, D10H6, R6H6 and D6.

The method of the invention preferably further comprises (d) purifying the hetero-oligomeric pore comprising the two different monomers in the specific stoichiometric ratio using the tag. For instance, consecutive histidine (H) residues (a hist-tag) may be used to purify the two different monomers in the specific stoichiometric ratio. For instance, if the hetero-oligomeric pore comprises 8 monomers, the specific stoichiometric ratio of the first different monomer to the second different monomer is 7:1 and the second different monomer is genetically fused to a his-tag, the his-tag may be used to purify pores containing the first and second different monomers in the ratio of 7:1. Pores containing the first and second different monomers in the ratio 8:0 if the his-tag is used and the elution concentration of the his-tag can be designed such that the different monomers are not purified in the ratio of 6:2.

The second different monomer is preferably genetically fused to the BasTL sequence (SEQ ID NO: 26) or a fragment thereof. This may increase or decrease the ability of the second different monomer to oligomerise with itself compared with the absence of the BasTL sequence (SEQ ID NO: 26) or a fragment thereof. The fragment may be any length, such as 40 or more, 50 or more, 60 or more, 70 or more, 80 or more or 90 or more amino acids in length. The fragment preferably comprises consecutive amino acids in the BasTL sequence (SEQ ID NO: 26). The fragment is preferably formed by deletion of 20 amino acids or 40 amino acids from the carboxy (C) terminus end. The BasTL sequence or fragment thereof is preferably genetically fused at the carboxy (C) terminus of the second different monomer. The BasTL sequence or fragment thereof is preferably genetically fused at the carboxy (C) terminus of the second different monomer and separates a tag as defined above from the second different monomer. For instance, the second different monomer may have the following structure from N to C: Monomer-SEQ ID NO: 26-H6.

### Hetero-Oligomeric Pores

The invention provides hetero-oligomeric pores produced using the method of the invention. Any of the embodiments discussed above apply to the pores of the invention.

The pores have improved polynucleotide reading properties i.e. display improved polynucleotide capture and nucleotide discrimination. In particular, the pores of the invention capture nucleotides and polynucleotides more easily than the wild type. In addition, the pores of the invention display an increased current range, which makes it easier to discriminate between different nucleotides, and a reduced variance of states, which increases the signal-to-noise ratio. In addition, the number of nucleotides contributing to the current as the polynucleotide moves through pores constructed from the mutants is decreased. This makes it easier to identify a direct relationship between the observed current as the polynucleotide moves through the pore and the polynucleotide sequence. The pores of the invention may also display improved movement of the polynucleotide as discussed in more detail below.

The pores of the invention can also discriminate between different nucleotides under a range of conditions. In particular, the pores will discriminate between nucleotides under conditions that are favourable to the characterising, such as sequencing, of nucleic acids. The extent to which the pores of the invention can discriminate between different nucleotides can be controlled by altering the applied potential, the salt concentration, the buffer, the temperature and the presence of additives, such as urea, betaine and DTT. This allows the function of the pores to be fine-tuned, particularly when sequencing. This is discussed in more detail below. The pores of the invention may also be used to identify polynucleotide polymers from the interaction with one or more monomers rather than on a nucleotide by nucleotide basis.

In general, the main advantage of hetero-oligomeric pores over homo-oligomeric pores is the ability to change, alter or mutate one or more of the monomers of the pore relative to (or differently from) the other monomers. Any part of the one or more monomers may be altered or mutated, for instance the part of the one or more monomers which forms the part of the pore which interacts with the polynucleotide or the part of the one or more monomers which forms the top of the pore and which interacts with a polynucleotide binding protein. By mutating the pore asymmetrically (i.e. differently in one or more of the monomers), the range, shape and level of the current signal obtained from the pore, as well as the signal to noise ratio, can be altered in a way that cannot be achieved by mutating all of the monomers.

A pore of the invention may be isolated, substantially isolated, purified or substantially purified. A pore of the invention is isolated or purified if it is completely free of any other components, such as lipids or other pores. A pore is substantially isolated if it is mixed with carriers or diluents which will not interfere with its intended use. For instance, a pore is substantially isolated or substantially purified if it is present in a form that comprises less than 10%, less than 5%, less than 2% or less than 1% of other components, such as triblock copolymers, lipids or other pores. Alternatively, a pore of the invention may be present in a membrane. Suitable membranes are discussed below.

A pore of the invention may be present as an individual or single pore. Alternatively, a pore of the invention may be present in a homologous or heterologous population of two or more pores.

The pores of the invention are hetero-oligomeric. The hetero-oligomeric pore contains sufficient monomers to form the pore. The monomers may be of any type. The pore typically comprises at least 3, at least 4, at least 5, at least 6, at least 7, at least 8, at least 9, at least 10 monomers, at least 11 monomers, at least 12 monomers, at least 13 monomers or at least 14 monomers, such as 3, 4, 5, 6, 7, 8, 9, 10, 11, 12, 13 or 14 monomers. The pore preferably comprises seven, eight or nine monomers.

A pore is hetero-oligomeric if at least one of the monomers differs from the rest of the monomers in the pore. The at least one monomer may be different in any way. The at least one monomer is typically different from the others on the basis of its amino acid sequence. The at least one monomer may be different from the rest of the monomers on the basis of 1, 2, 3, 4, 5, 10, 15, 20 or more amino acid differences. Two, three or four of the monomers may be the same and different from the rest of the monomers in the pore. All of the monomers in the pore may be different from one another. Preferably, only one monomer is different from the rest of the monomers in the pore, i.e. the rest of the monomers are the same.

The hetero-oligomeric pore is preferably derived from Msp and comprises a narrowing having a net negative charge. Such pores are disclosed in the UK Application 1502809.5 being co-filed with this application. Preferably, only one monomer is different from the rest of the monomers in the pore and the only one monomer differs from the rest of the monomers on the basis of one or more negatively charged amino acids in its region which forms part of the narrowing.

The narrowing has a net negative charge. The narrowing is typically the narrowest part of the channel of the pore. The narrowing of the pore is typically not part of the cap region or the barrel region. The internal diameter of the narrowing (i.e. the diameter of the channel through the narrowing) is typically about 25 ångströms (Å) or less, such as about 22 Å or less, about 20 Å or less, about 18 Å or less, about 16 Å or less, about 14 Å or less or about 12 Å or less.

The narrowing has a net negative charge. The narrowing has a net negative charge at physiological pH. The narrowing typically has a net negative charge when the pH is in the range of 2 to 12, 2.5 to 11, 3 to 10, more preferably in the range of 4 to 9, 5 to 8.5 or even more preferably in the range of 6 to 8 or 6.5 to 7.5.

The net charge of the narrowing can be measured using methods known in the art. For instance, the net charge of the narrowing can be calculated at a specific pH using routine methods.

The narrowing typically comprises one or more negatively charged amino acids. The narrowing may comprise any number of negatively charged amino acids, such as 1, 2, 3, 4, 5, 6, 7, 8, 9, 10, 11, 12 or more negatively charged amino acids. The narrowing preferably comprises one, two, three or four negatively charged amino acids. A negatively charged amino acid is an amino acid with a net negative charge. Negatively charged amino acids include, but are not limited to, aspartic acid (D) and glutamic acid (E). The skilled person can also design other negatively charged amino acids. For instance, a cysteine (C) can be modified with a negatively charged molecule.

If the narrowing contains more than one negatively charged amino acid, such as two, three or four negatively charged amino acids, they are preferably in different monomers, i.e. are not all in the same monomer. For instance, if there are two negatively charged amino acids, each may be in a different monomer. If there are three negatively charged amino acids, they may be in two or three different monomers. If the narrowing contains more than one negatively charged amino acid, such as two, three or four negatively charged amino acids, they may be in the same monomer.

The remaining amino acids in the narrowing are preferably not charged. Amino acids which are not charged are typically uncharged, non-polar and/or aromatic amino acids. The uncharged amino acids, non-polar amino acids and/or aromatic amino acids can be naturally occurring or non-naturally-occurring. They may be synthetic or modified. Uncharged amino acids have no net charge. Suitable uncharged amino acids include, but are not limited to, cysteine (C), serine(S), threonine (T), methionine (M), asparagines (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y). Any number and combination of these amino acids may be present in the narrowing in addition to one or more negatively charged amino acids.

If the narrowing comprises one or more positively charged amino acids, there are preferably fewer positively charged amino acids than negatively charged amino acids in the narrowing. The narrowing preferably does not comprise any positively charged amino acids. Positively charged amino acids include, but are not limited to, histidine (H), lysine (K) and arginine (R).

The monomers in the pore are preferably approximately the same length or are the same length. The barrels of the monomers in the pore are preferably approximately the same length or are the same length. Length may be measured in number of amino acids and/or units of length. Barrel deletions are discussed in more detail below. The monomers in the pore preferably have the same number of amino acids deleted from positions 72 to 82 and/or positions 111 to 121 of SEQ ID NO: 2. As discussed above, one or more of the monomers may be attached to a tag or BasTL sequence or fragment thereof which makes it longer than the other monomers in the pore.

In all the embodiments discussed above, one or more, such as 2, 3, 4, 5, 6, 7, 8, 9 or 10, of the mutant monomers may be chemically modified as discussed below.

The pore preferably comprises eight monomers each comprising the sequence shown in SEQ ID NO: 2 or a variant thereof. SEQ ID NO: 2 is the wild-type MspA monomer. The pore preferably comprises two different monomers each comprising the sequence shown in SEQ ID NO: 2 or a variant thereof and wherein the specific stoichiometric ratio of the first different monomer to the second different monomer is 7:1 or 8:1, most preferably 7:1.

A variant of SEQ ID NO: 2 is a polypeptide that has an amino acid sequence which varies from that of SEQ ID NO: 2 and which retains its ability to form a pore. A variant or each variant may comprise only amino acid substitutions compared with SEQ ID NO: 2 or may comprise amino acid deletions. Variants are discussed in more detail below.

The hetero-oligomeric pore may comprise any number of monomers comprising SEQ ID NO: 2, such as 1, 2, 3 or 4 monomers comprising SEQ ID NO: 2. The pore may comprise any number of monomers comprising a variant of SEQ ID NO: 2, such as 1, 2, 3, 4, 5, 6, 7 or 8 monomers comprising a variant of SEQ ID NO: 2. All of the monomers in the pore preferably comprise a variant of SEQ ID NO: 2, i.e. all of the monomers in the pore have been modified in some way compared with the wild type sequence shown in SEQ ID NO: 2. Suitable modifications are discussed below.

The pore may comprise seven first different monomers each comprising a variant of SEQ ID NO: 2 comprising D90N, D91N and D93N and one second different monomer comprising SEQ ID NO: 2.

Amino acids 83 to 111 in SEQ ID NO: 2 contribute to the narrowing of a pore comprising monomers comprising SEQ ID NO: 2. The narrowing in a pore of the invention preferably comprises the amino acids in each monomer which correspond to positions 83 to 111 of SEQ ID NO: 2. Amino acids in a monomer comprising a variant of SEQ ID NO: 2 correspond to positions in SEQ ID NO: 2 with which they align. Any method of alignment may be used, including any of those discussed below. If only amino acids substitutions are made to SEQ ID NO: 2 to produce a variant, the amino acids in the variant which correspond to positions 83 to 111 in SEQ ID NO: 2 will be numbered 83 to 111, i.e. position 90 in the variant corresponds with position 90 in SEQ ID NO: 2. If parts of SEQ ID NO: 2 are deleted to produce the variant or amino acids are added to SEQ ID NO: 2 to form the variant, the amino acids in the variant which correspond to positions 83 to 111 in SEQ ID NO: 2 will not be numbered 83 to 111. By way of illustration, positions 71 to 111 of SEQ ID NO: 2 (i.e. SEQ ID NO: 3) are shown below. The corresponding part of a variant of SEQ ID NO: 2, which only differs from SEQ ID NO: 2 by the deletion of positions 72 and 73 (from the barrel) and the substitutions D91N and D93N (in bold), is shown in SEQ ID NO: 4.

(SEQ ID NO: 3)
..PWSLGVGINFSYTTPNILIDDGDITAPPFGLNSVITPNLFPGVS
ISADLGN...
(SEQ ID NO: 4)
...P--LGVGINFSYTTPNILIDNGNITAPPFGLNSVITPNLFPGVS
ISADLGN...

Positions D90, N91 and N93 in the variant (underlined in SEQ ID NO: 4) correspond to positions D88, D89 and D91 in SEQ ID NO: 2 respectively (because amino acids 72 and 73 in SEQ ID NO: 2 are deleted from the variant). Based on this, a skilled person can determine which amino acids in a variant correspond with positions 83 to 111 in SEQ ID NO: 2.

The narrowing more preferably comprises the amino acids in each monomer which correspond to positions 88, 90, 91, 92, 93, 102, 103 and 105 of SEQ ID NO: 2. If a monomer or each monomer comprises a variant of SEQ ID NO: 2 in which only amino acid substitutions are made, the narrowing preferably comprises the amino acids at positions 88, 90, 91, 92, 93, 102, 103 and 105 in the variant.

The narrowing more preferably comprises the amino acids in each monomer which correspond to positions 90, 91, 93 and 105 of SEQ ID NO: 2. If a monomer or each monomer comprises a variant of SEQ ID NO: 2 in which only amino acids substitutions are made, the narrowing more preferably comprises the amino acids at positions 90, 91, 93 and 105 in the variant.

The second different monomer preferably differs from the first different monomer by comprising a negatively charged amino acid at one or more of the positions which correspond to positions 88, 90, 91, 92, 93, 102, 103 and 105 of SEQ ID NO: 2. A negatively charged amino acid may be present at any number and combination of the positions which correspond to positions 88, 90, 91, 92, 93, 102, 103 and 105 of SEQ ID NO: 2. Preferred combinations are discussed in more detail below. Negatively charge amino acids are discussed above. If each monomer in the pore comprises a variant of SEQ ID NO: 2 in which only amino acids substitutions are made, the second different monomer preferably differs from the first different monomer by comprising a negatively charged amino acid at one or more of the positions 88, 90, 91, 92, 93, 102, 103 and 105.

The second different monomer preferably differs from the first different monomer by comprising a negatively charged amino acid at one or more of the positions which correspond to positions 90, 91, 93 and 105 of SEQ ID NO: 2. A negatively charged amino acid may be present at any number and combination of positions which correspond to positions 90, 91, 93 and 105 of SEQ ID NO: 2, namely (i) 90; (ii) 91; (iii) 93; (iv) 105; (v) 90 and 91; (vi) 90 and 93; (vii) 90 and 105; (viii) 91 and 93; (ix) 91 and 105; (x) 93 and 105; (xi) 90, 91 and 93; (xii) 90, 91 and 105; (xiii) 90, 93 and 105; (xiv) 91, 93 and 105; or (xv) 90, 91, 93 and 105. The second different monomer preferably differs from the first different monomer by comprising a negatively charged amino acid at the position(s) which correspond(s) to position(s) (x) 90; (xi) 91; (xii) 93; (xiii) 90 and 91; (xiv) 90 and 93; (xv) 91 and 93; (xvi) 105; (xvii) 90 and 105; or (xviii) 90, 91 and 105 of SEQ ID NO: 2. Negatively charged amino acids are discussed above. If each monomer in the pore comprises a variant of SEQ ID NO: 2 in which only amino acid substitutions are made, the second different monomer preferably differs from the first different monomer by comprising a negatively charged amino acid at one or more of the positions 90, 91, 93 and 105. The one or more monomers may differ by comprising a negatively charged amino acid at any combination of positions 90, 91, 93 and 105 discussed in (i) to (xv) or (x) to (xviii) above.

Preferably, the second different monomer differs from the first different monomer by comprising one or more negatively charged amino acids, such as 1, 2, 3 or 4 negatively charged amino acids, in its narrowing forming region. For instance, the second different monomer preferably differs from the first different monomer by comprising a negatively charged amino acid at one or more of the positions which correspond to positions 90, 91, 93 and 105 of SEQ ID NO: 2. The second different monomer may comprise a negatively charged amino acid at any number and combination of the positions which correspond to positions 90, 91, 93 and 105 of SEQ ID NO: 2 discussed above in (i) to (xv) or (x) to (xviii).

In any of the embodiments discussed above, the first different monomer preferably comprises an amino acid which is not charged at the position(s) which correspond(s) to position(s) 90 and/or 91 of SEQ ID NO: 2. The first different monomer preferably comprises an amino acid which is not charged at the position(s) which correspond(s) to position(s) 90, 91 and/or 93 of SEQ ID NO: 2. If each monomer in the pore comprises a variant of SEQ ID NO: 2 in which only amino acids substitutions are made, the first different monomer preferably comprises an amino acid which is not charged at position(s) 90 and/or 91 or positions 90, 91 and/or 93. Any of the amino acids which are not charged and which are discussed above may be present. The amino acid is preferably asparagine (N) or glutamine (Q).

A preferred pore of the invention is one in which:
(a) the second different monomer comprises an aspartic acid (D) at the position which corresponds to position 90 of SEQ ID NO: 2 and the first different monomer comprises an asparagine (N) at the position which corresponds to position 90 of SEQ ID NO: 2;
(b) the second different monomer comprises an aspartic acid (D) at the position which corresponds to position 91 of SEQ ID NO: 2 and the first different monomer comprises an asparagine (N) at the position which corresponds to position 91 of SEQ ID NO: 2;
(c) the second different monomer comprises an aspartic acid (D) at the positions which correspond to positions 90 and 91 of SEQ ID NO: 2 and the first different monomer comprises an asparagine (N) at the positions which correspond to positions 90 and 91 of SEQ ID NO: 2;
(d) the second different monomer comprises a glutamic acid (E) at the position which corresponds to position 90 of SEQ ID NO: 2 and the first different monomer comprises an asparagine (N) at the position which corresponds to position 90 of SEQ ID NO: 2;
(e) the second different monomer comprises a glutamic acid (E) at the position which corresponds to position 91 of SEQ ID NO: 2 and the first different monomer comprises an asparagine (N) at the position which corresponds to position 91 of SEQ ID NO: 2;
(f) the second different monomer comprises an glutamic acid (E) at the positions which correspond to positions 90 and 91 of SEQ ID NO: 2 and the first different monomer comprises an asparagine (N) at the positions which correspond to positions 90 and 91 of SEQ ID NO: 2;
(g) the second different monomer comprises an aspartic acid (D) at the position which corresponds to position 90 of SEQ ID NO: 2 and the first different monomer comprises a glutamine (Q) at the position which corresponds to position 90 of SEQ ID NO: 2;
(h) the second different monomer comprises an aspartic acid (D) at the position which corresponds to position 91 of SEQ ID NO: 2 and the first different monomer comprises an glutamine (Q) at the position which corresponds to position 91 of SEQ ID NO: 2;
(i) the second different monomer comprises an aspartic acid (D) at the positions which correspond to positions 90 and 91 of SEQ ID NO: 2 and the first different monomer comprises an glutamine (Q) at the positions which correspond to positions 90 and 91 of SEQ ID NO: 2;
(j) the second different monomer comprises a glutamic acid (E) at the position which corresponds to position 90 of SEQ ID NO: 2 and the first different monomer comprises an glutamine (Q) at the position which corresponds to position 90 of SEQ ID NO: 2;
(k) the second different monomer comprises a glutamic acid (E) at the position which corresponds to position 91 of SEQ ID NO: 2 and the first different monomer comprises an glutamine (Q) at the position which corresponds to position 91 of SEQ ID NO: 2;
(l) the second different monomer comprises an glutamic acid (E) at the positions which correspond to positions 90 and 91 of SEQ ID NO: 2 and the first different monomer comprises an glutamine (Q) at the positions which correspond to positions 90 and 91 of SEQ ID NO: 2;
(m) the second different monomer comprises an aspartic acid (D) at the position which corresponds to position 88 of SEQ ID NO: 2 and the first different monomer comprises an asparagine (N) at the position which corresponds to position 88 of SEQ ID NO: 2;
(n) the second different monomer comprises an aspartic acid (D) at the position which corresponds to position 88 of SEQ ID NO: 2 and the first different monomer comprises a glutamine (Q) at the position which corresponds to position 88 of SEQ ID NO: 2;
(o) the second different monomer comprises an aspartic acid (D) at the position which corresponds to position 103 of SEQ ID NO: 2 and the first different monomer comprises a serine(S) at the position which corresponds to position 103 of SEQ ID NO: 2;
(p) the second different monomer comprises an aspartic acid (D) at the position which corresponds to position 105 of SEQ ID NO: 2 and the first different monomer comprises an isoleucine (I) at the position which corresponds to position 105 of SEQ ID NO: 2;
(q) the second different monomer comprises an aspartic acid (D) at the positions which correspond to positions 88 and 90 of SEQ ID NO: 2 and the first different monomer comprises an asparagine (N) at the positions which correspond to positions 88 and 90 of SEQ ID NO: 2;
(r) the second different monomer comprises an aspartic acid (D) at the positions which correspond to positions 88 and 90 of SEQ ID NO: 2 and the first different monomer comprises a glutamine (Q) at the positions which correspond to positions 88 and 90 of SEQ ID NO: 2; (s) the second different monomer comprises an aspartic acid (D) at the positions which correspond to positions 90 and 103 of SEQ ID NO: 2 and the first different monomer comprises an asparagine (N) and serine (S) at the positions which correspond to positions 90 and 103 of SEQ ID NO: 2 respectively;
(t) the second different monomer comprises an aspartic acid (D) at the positions which correspond to positions 90 and 103 of SEQ ID NO: 2 and the first different monomer comprises a glutamine (Q) and serine(S) at the positions which correspond to positions 90 and 103 of SEQ ID NO: 2 respectively;
(u) the second different monomer comprises an aspartic acid (D) at the positions which correspond to positions 90 and 105 of SEQ ID NO: 2 and the first different monomer comprises an asparagine (N) and isoleucine (I) at the positions which correspond to positions 90 and 105 of SEQ ID NO: 2 respectively;
(v) the second different monomer comprises an aspartic acid (D) at the positions which correspond to positions 90 and 105 of SEQ ID NO: 2 and the first different monomer comprises a glutamine (Q) and isoleucine (I) at the positions which correspond to positions 90 and 105 of SEQ ID NO: 2 respectively;
(w) the second different monomer comprises an aspartic acid (D) at the positions which correspond to positions 90 and 93 of SEQ ID NO: 2 and the first different monomer comprises an asparagine (N) at the positions which correspond to positions 90 and 93 of SEQ ID NO: 2;
(x) the second different monomer comprises an aspartic acid (D) at the positions which correspond to positions 90 and 93 of SEQ ID NO: 2 and the first different monomer comprises a glutamine (Q) at the positions which correspond to positions 90 and 93 of SEQ ID NO: 2;
(y) the second different monomer comprises an aspartic acid (D) at the positions which correspond to positions 91 and 93 of SEQ ID NO: 2 and the first different monomer comprises an asparagine (N) at the positions which correspond to positions 91 and 93 of SEQ ID NO: 2;
(z) the second different monomer comprises an aspartic acid (D) at the positions which correspond to positions 91 and 93 of SEQ ID NO: 2 and the first different monomer comprises a glutamine (Q) at the positions which correspond to positions 91 and 93 of SEQ ID NO: 2;
(aa) the second different monomer comprises an aspartic acid (D) at the position which corresponds to position 93 of SEQ ID NO: 2 and the first different monomer comprises an asparagine (N) at the position which corresponds to position 93 of SEQ ID NO: 2;
(ab) the second different monomer comprises an aspartic acid (D) at the position which corresponds to position 93 of SEQ ID NO: 2 and the first different monomer comprises a glutamine (Q) at the position which corresponds to position 93 of SEQ ID NO: 2;
(ac) the second different monomer comprises an aspartic acid (D) at the position which corresponds to position 90 of SEQ ID NO: 2 and the first different monomer comprises an aspartic acid (D) at the position which corresponds to position 93 of SEQ ID NO: 2;
(ad) the second different monomer comprises an aspartic acid (D) at the positions which correspond to positions 90 and 93 of SEQ ID NO: 2 and the first different monomer comprises an aspartic acid (D) at the position which corresponds to position 93 of SEQ ID NO: 2;
(ae) the second different monomer comprises an aspartic acid (D) at the positions which correspond to positions 91 and 93 of SEQ ID NO: 2 and the first different monomer comprises an aspartic acid (D) at the position which corresponds to position 93 of SEQ ID NO: 2;
(af) the second different monomer comprises an aspartic acid (D) at the position which corresponds to position 90 of SEQ ID NO: 2 and the first different monomer comprises an aspartic acid (D) at the position which corresponds to position 93 of SEQ ID NO: 2;
(ag) the second different monomer comprises an aspartic acid (D) at the positions which correspond to positions 88 and 105 of SEQ ID NO: 2 and the first different monomer comprises an asparagine (N) and isoleucine (I) at the positions which correspond to positions 88 and 105 of SEQ ID NO: 2 respectively; or
(ah) the second different monomer comprises an aspartic acid (D) at the positions which correspond to positions 88 and 105 of SEQ ID NO: 2 and the first different monomer comprises a glutamine (Q) and isoleucine (I) at the positions which correspond to positions 88 and 105 of SEQ ID NO: 2 respectively.

In any of (a) to (ah) above, if each monomer in the pore comprises a variant of SEQ ID NO: 2 in which only amino acids substitutions are made, the positions which correspond to positions in SEQ ID NO: 2 have the same number as the positions in SEQ ID NO: 2. For instance, if in (a) each monomer in the pore comprises a variant of SEQ ID NO: 2 in which only amino acids substitutions are made, the second different monomer comprises an aspartic acid (D) at position 90 and the first different monomer comprises an asparagine (N) at position 90.

A variant of SEQ ID NO: 2 is a polypeptide that has an amino acid sequence which varies from that of SEQ ID NO: 2 and which retains its ability to form a pore. The ability of a variant to form a pore can be assayed using any method known in the art. For instance, the variant may be inserted into an amphiphilic layer along with other appropriate subunits and its ability to oligomerise to form a pore may be determined. Methods are known in the art for inserting subunits into membranes, such as amphiphilic layers. For example, subunits may be suspended in a purified form in a solution containing a triblock copolymer membrane such that it diffuses to the membrane and is inserted by binding to the membrane and assembling into a functional state. A variant may be modified in various ways.

A pore of the invention may comprise any number of monomers each comprising a variant of SEQ ID NO: 2, such as 1, 2, 3, 4, 5, 6, 7 or 8 monomers each comprising a variant of SEQ ID NO: 2. In a preferred embodiment, all of the monomers in the pore comprise a variant of SEQ ID NO: 2.

The or each variant of SEQ ID NO: 2 preferably comprises a mutation or substitution at one or more of the positions which correspond to positions G75, G77, L88, D118, Q126, D134 and E139 of SEQ ID NO: 2. If the or each monomer in the pore comprises a variant of SEQ ID NO: 2 in which only amino acids substitutions are made, the or each variant preferably comprises a mutation or substitution at one or more of positions G75, G77, L88, D118, Q126, D134 and E139. The or each variant more preferably comprises one or more of G75S, G77S, L88N, D118R, Q126R, D134R and E139K. The purpose of these mutations is discussed in more detail below.

### Rigidity

The or each variant of SEQ ID NO: 2 preferably comprises proline (P) at the position which corresponds to position 108 in SEQ ID NO: 2. If the or each monomer in the pore comprises a variant of SEQ ID NO: 2 in which only amino acids substitutions are made, the or each variant preferably comprises P at position 108.

### Barrel Deletions

In the or each variant of SEQ ID NO: 2, (a) 2, 4, 6, 8 or 10 of the amino acids which correspond to positions 72 to 82 of SEQ ID NO: 2 have preferably been deleted and (b) 2, 4, 6, 8 or 10 of the amino acids which correspond to positions 111 to 121 of SEQ ID NO: 2 have preferably been deleted. In other words, 2, 4, 6, 8 or 10 amino acids are preferably deleted from the downward strand (positions 72 to 82) and the upward strand (positions 111 to 121) of the barrel region of SEQ ID NO: 2 when forming the variant. Deletion of amino acids from positions 72 to 82 and 11 to 121 alters the numbering of the subsequent amino acids in the variant as discussed above.

The number of amino acids deleted from positions 72 to 82 may be different from the number of amino acids deleted from positions 111 to 121. The number of amino acids deleted from positions 72 to 82 is preferably the same as the number of amino acids deleted from positions 111 to 121.

Any combination of amino acids from positions 72 to 82 and amino acids from positions 111 to 121 may be deleted. The majority of the amino acids in the downward and upwards strands of the barrel in SEQ ID NO: 2 alternate between hydrophobic and hydrophilic. The hydrophobic amino acids are selected from tryptophan (W), leucine (L), valine (V), isoleucine (I), phenylalanine (F) and tyrosine (Y). The hydrophilic amino acids are selected from serine (S), glycine (G), asparagine (N), proline (P) and aspartic acid (D). The alternation between hydrophobic and hydrophilic amino acids results in the beta-sheet which forms part of the barrel of the pore.

The amino acids from positions 72 to 82 remaining after deletion (i.e. after 2, 4, 6, 8 or 10 amino acids have been deleted from positions 72 to 82) preferably comprise 3, 5, 7 or 9 consecutive amino acids which alternate between hydrophobic and hydrophilic.

The amino acids from positions 111 to 121 remaining after deletion (i.e. after 2, 4, 6, 8 or 10 amino acids have been deleted from positions 111 to 121) preferably comprise 3, 5, 7 or 9 consecutive amino acids which alternate between hydrophobic and hydrophilic.

The amino acids deleted from positions 72 to 82 may correspond to the amino acids deleted from positions 111 to 121 as shown in Table 3 below. For instance, if L74 and G75 are deleted from positions 72 to 82, D118 and L119 may be deleted from positions 111 to 121.

TABLE 3
Corresponding amino acids in the barrel of SEQ ID NO: 2

| Position in (a) | Corresponding position in (b) |
|---|---|
| W72 | N121 |
| S73 | G120 |
| L74 | L119 |
| G75 | D118 |
| V76 | A117 |
| G77 | S116 |
| I78 | I115 |
| N79 | S114 |
| F80 | V113 |
| S81 | G112 |
| Y82 | P111 |

One or more positions of the amino acids that have been deleted from positions 72 to 82 may not correspond to the one or more positions of the amino acids that have been deleted from positions 111 to 121 as shown in Table 3. For instance, if L74 and G75 are deleted from positions 72 to 82, A117 and D118 may be deleted from positions 111 to 121.

The positions of (all of) the amino acids that have been deleted from positions 72 to 82 may not correspond to the positions of (all of) the amino acids that have been deleted from positions 111 to 121 as shown in Table 3. For instance, if L74 and G75 are deleted from positions 72 to 82, 1115 and S116 may be deleted from positions 111 to 121.

The amino acids deleted from positions 72 to 82 are preferably consecutive. The amino acids deleted from positions 111 to 121 are preferably consecutive. The amino acids deleted from positions 72 to 82 and the amino acids deleted from positions 111 to 121 are preferably consecutive.

The or each variant of the sequence shown in SEQ ID NO: 2 is preferably one in which (i) L74, G75, D118 and L119 have been deleted, (ii) G75, V76, A117 and D118 have been deleted, (iii) V76, G77, S116 and A117 have been deleted, (iv) G77, I78, I115 and S116 have been deleted, (v) I78, N79, S114 and I115 have been deleted, (vi) N79, F80, V113 and S114 have been deleted or (vii) F80, S81, G112 and V113 have been deleted. The or each variant of the sequence shown in SEQ ID NO: 2 is preferably one in which L74, G75, V76, G77, S116, A117, D118 and L119 have been deleted. The or each variant of the sequence shown in SEQ ID NO: 2 is preferably one in which L74, G75, N79, F80, V113, S114, D118 and L119 have been deleted or L74, G75, F80, S81, G112, V113, D118 and L119 have been deleted.

The skilled person can identify other combinations of amino acids that may be deleted in accordance with the invention.

### Positions 90 and 91

In SEQ ID NO: 2, amino acids 90 and 91 are both aspartic acid (D). A variant may comprise a negatively charged amino acid, such as aspartic acid (D) or glutamic acid (E), at the position(s) which correspond(s) to position(s) 90 and/or 91 of SEQ ID NO: 2 if the monomer comprising the variant is contributing to the negative charge in the narrowing. If the variant comprises glutamic acid (E) or a non-natural negatively charged amino acid at the position(s) which correspond(s) to position(s) 90 and/or 91 of SEQ ID NO: 2, it or they may be introduced by substitution. If a monomer comprises a variant of SEQ ID NO: 2 in which only amino acids substitutions are made, the variant may comprise a negatively charged amino acid, such as aspartic acid (D) or glutamic acid (E), at the position(s) 90 and/or 91.

A variant may not comprise a negatively charged amino acid at the position(s) which corresponds to position(s) 90 and/or 91 of SEQ ID NO: 2 if the monomer comprising the variant is not contributing to the negative charge in the narrowing. A variant may comprise any of the amino acids which are not charged and which are discussed above at the position(s) which corresponds to position(s) 90 and/or 91 of SEQ ID NO: 2. If a monomer comprises a variant of SEQ ID NO: 2 in which only amino acids substitutions are made, the variant may not comprise a negatively charged amino acid at the position(s) which corresponds to position(s) 90 and/or 91. The variant preferably may comprise serine(S), glutamine (Q), leucine (L), methionine (M), isoleucine (I), alanine (A), valine (V), glycine (G), phenylalanine (F), tryptophan (W), tyrosine (Y), histidine (H), threonine (T), arginine (R), lysine (K), asparagine (N) or cysteine (C) at the position(s) which corresponds to position(s) 90 and/or 91 of SEQ ID NO: 2 or at position(s) 90 and/or 91. Any combinations of these amino acids at positions 90 and 91 are envisaged by the invention. The variant preferably comprises asparagine (N) or glutamine (Q) at the position(s) which corresponds to position(s) 90 and/or 91 of SEQ ID NO: 2 or at position(s) 90 and/or 91. These amino acids are preferably inserted at position 90 and/or 91 by substitution.

### Position 93

In wild-type MspA, amino acid 93 is aspartic acid (D). A variant may comprise a negatively charged amino acid, such as aspartic acid (D) or glutamic acid (E), at the position which corresponds to position 93 of SEQ ID NO: 2 if the monomer comprising the variant is contributing to the negative charge in the narrowing. If the variant comprises glutamic acid (E) or a non-natural negatively charged amino acid at the position which corresponds to position 93 of SEQ ID NO: 2, it may be introduced by substitution. If a monomer in the pore comprises a variant of SEQ ID NO: 2 in which only amino acid substitutions are made, the variant may comprise a negatively charged amino acid, such as aspartic acid (D) or glutamic acid (E), at the position 93.

A variant may not comprise a negatively charged amino acid at the position which corresponds to position 93 of SEQ ID NO: 2 if the monomer comprising the variant is not contributing to the negative charge in the narrowing. If a monomer in the pore comprises a variant of SEQ ID NO: 2 in which only amino acids substitutions are made, the variant may not comprise a negatively charged amino acid, such as aspartic acid (D) or glutamic acid (E), at the position 93. A variant may comprise any of the amino acids which are not charged and which are discussed above at the position which corresponds to position 93 of SEQ ID NO: 2 or at position 93. The variant preferably may comprise serine(S), glutamine (Q), leucine (L), methionine (M), isoleucine (I), alanine (A), valine (V), glycine (G), phenylalanine (F), tryptophan (W), tyrosine (Y), histidine (H), threonine (T), arginine (R), lysine (K), asparagine (N) or cysteine (C) at the position which corresponds to position 93 of SEQ ID NO: 2 or at position 93. The variant preferably comprises asparagine (N) at the position which corresponds to position 93 of SEQ ID NO: 2 or at position 93. These amino acids are preferably inserted at position 93 by substitution.

### Cap Forming Region

In SEQ ID NO: 2, amino acids 1 to 72 and 122 to 184 form the cap of the pore. Of these amino acids, V9, Q12, D13, R14, T15, W40, I49, P53, G54, D56, E57, E59, T61, E63, Y66, Q67, 168, F70, P123, I125, Q126, E127, V128, A129, T130, F131, S132, V133, D134, S136, G137, E139, V144, H148, T150, V151, T152, F163, R165, 1167, S169, T170 and S173 face inwards into the channel of the pore.

### Barrel Forming Region

In SEQ ID NO: 2, amino acids 72 to 82 and 112 to 121 form the barrel of the pore. Of these amino acids, S73, G75, G77, N79, S81, G112, S114, S116, D118 and G120 face inwards into the channel of the pore. S73, G75, G77, N79, S81 face inwards in the downwards strand and G112, S114, S116, D118 and G120 face inwards in the upwards strand.

### Decreased Net Negative Charge

The or each variant preferably comprises one or more modifications which decrease the net negative charge of the inward facing amino acids in the cap forming region and/or the barrel forming region of the monomer. The variant preferably comprises two or more modifications which decrease the net negative charge of the inward facing amino acids in the cap forming region and/or the barrel forming region of the monomer. Any such modifications to the barrel forming region are in addition to the deletions of the invention discussed above.

The variant may comprise any number of modifications, such as 1 or more, 2 or more, 3 or more, 4 or more, 5 or more, 6 or more, 7 or more, 8 or more, 9 or more, 10 or more, 15 or more, 20 or more, 30 or more, or 40 or more modifications.

The net negative charge may be decreased by any means known in the art. The net negative charge is decreased in a manner that does not interfere with the ability of the mutant monomer to form a pore. This can be measured as discussed above.

The net negative charge of the inward facing amino acids in the cap forming region and/or the barrel forming region may be decreased. This means that the inward facing amino acids in the cap forming region and/or the barrel forming region comprise fewer negatively charged amino acids than in SEQ ID NO: 2 and/or comprises more positively charged amino acids than in SEQ ID NO: 2. The one or more modifications may lead to a net positive charge in the inward facing amino acids in the cap forming region and/or the barrel forming region

The net charge can be measured using methods known in the art. For instance, the net charge of the inward facing amino acids in the cap forming region and/or the barrel forming region may be calculated using routine methods.

The one or more modifications are preferably one or more deletions of negatively charged amino acids. Removal of one or more negatively charged amino acids reduces the net negative charge of the inward facing amino acids in the cap forming region and/or barrel forming region. A negatively charged amino acid is an amino acid with a net negative charge. Negatively charged amino acids include, but are not limited to, aspartic acid (D) and glutamic acid (E). Methods for deleting amino acids from proteins, such as MspA monomers, are well known in the art.

The one or more modifications are preferably one or more substitutions of negatively charged amino acids with one or more positively charged, uncharged, non-polar and/or aromatic amino acids. A positively charged amino acid is an amino acid with a net positive charge. The positively charged amino acid(s) can be naturally-occurring or non-naturally-occurring. The positively charged amino acid(s) may be synthetic or modified. For instance, modified amino acids with a net positive charge may be specifically designed for use in the invention. A number of different types of modification to amino acids are well known in the art.

Preferred naturally-occurring positively charged amino acids include, but are not limited to, histidine (H), lysine (K) and arginine (R). Any number and combination of H, K and/or R may be substituted for the inward facing amino acids in the cap forming region and/or barrel forming region.

The uncharged amino acids, non-polar amino acids and/or aromatic amino acids can be naturally occurring or non-naturally-occurring. They may be synthetic or modified. Uncharged amino acids have no net charge. Suitable uncharged amino acids include, but are not limited to, cysteine (C), serine(S), threonine (T), methionine (M), asparagines (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y). Any number and combination of these amino acids may be substituted into the inward facing amino acids in the cap forming region and/or the barrel forming region.

The one or more negatively charged amino acids are preferably substituted with alanine (A), valine (V), asparagine (N), glutamine (Q) or glycine (G). Preferred substitutions include, but are not limited to, substitution of D with A, substitution of D with V, substitution of D with N, substitution of D with Q and substitution of D with G.

The one or more modifications are preferably one or more introductions of positively charged amino acids. The introduction of positive charge decreases the net negative charge. The one or more positively charged amino acids may be introduced by addition or substitution. Any amino acid may be substituted with a positively charged amino acid. One or more uncharged amino acids, non-polar amino acids and/or aromatic amino acids may be substituted with one or more positively charged amino acids. Any number of positively charged amino acids may be introduced.

Wild-type MspA comprises a polar glutamine (Q) at position 126. The one or more modifications preferably reduce the net negative charge at the position in a variant which corresponds to position 126 in SEQ ID NO: 2. If the monomer comprises a variant of SEQ ID NO: 2 in which only amino acids substitutions are made, one or more modifications preferably reduce the net negative charge at the position in a variant which corresponds to position 126. The one or more modifications preferably increase the net positive charge at the position which corresponds to position 126 or at position 126. This can be achieved by replacing the polar amino acid at position 126 or an adjacent or a nearby inward facing amino acid with a positively charged amino acid. The or each variant preferably comprises a positively charged amino acid at the position which corresponds to position 126 or at position 126. The or each variant preferably comprises a positively charged amino acid at one or more of the positions which correspond to positions 123, 125, 127 and 128 in SEQ ID NO: 2 or at one or more of positions 123, 125, 127 and 128. The or each variant may comprise any number and combination of positively charged amino acids at the positions which correspond to positions 123, 125, 127 and 128 or at one or more of positions 123, 125, 127 and 128. The positively charged amino acid(s) may be introduced by addition or substitution.

The one or more modifications are preferably one or more introductions of positively charged amino acids which neutralise one or more negatively charged amino acids. The neutralisation of negative charge decreases the net negative charge. The one or more positively charged amino acids may be introduced by addition or substitution. Any amino acid may be substituted with a positively charged amino acid. One or more uncharged amino acids, non-polar amino acids and/or aromatic amino acids may be substituted with one or more positively charged amino acids. Any number of positively charged amino acids may be introduced. The number is typically the same as the number of negatively charged amino acids being neutralised.

The one or more positively charged amino acids may be introduced at any position in the cap forming region and/or the barrel forming region as long as they neutralise the negative charge of the one or more inward facing negatively charged amino acids. To effectively neutralise the negative charge in the cap forming region, there is typically 5 or fewer amino acids in the variant between each positively charged amino acid that is introduced and the negatively charged amino acid it is neutralising. There are preferably 4 or fewer, 3 or fewer or 2 or fewer amino acids in the cap forming region of the variant between each positively charged amino acid that is introduced and the negatively charged amino acid it is neutralising. There is more preferably two amino acids in the cap forming region of the variant between each positively charged amino acid that is introduced and the negatively charged amino acid it is neutralising. Each positively charged amino acid is most preferably introduced adjacent in the cap forming region of the variant to the negatively charged amino acid it is neutralising.

To effectively neutralise the negative charge in the barrel forming region, there is typically 5 or fewer inward facing amino acids between each positively charged amino acid that is introduced and the negatively charged amino acid it is neutralising. There is preferably 4 or fewer, 3 or fewer or 2 or fewer inward facing amino acids in the barrel forming region of the variant between each positively charged amino acid that is introduced and the negatively charged amino acid it is neutralising. There is more preferably one inward facing amino acid in the barrel forming region of the variant between each positively charged amino acid that is introduced and the negatively charged amino acid it is neutralising. Each positively charged amino acid is most preferably introduced at the inward facing position adjacent in the barrel forming region of the variant to the negatively charged amino acid it is neutralising.

SEQ ID NO: 2 comprises aspartic acid (D) at positions 118 and 134 and glutamic acid (E) at position 139. Amino acid 118 in each monomer is present within the barrel of the pore. The or each variant preferably comprises a positively charged amino acid at one or more of the positions which correspond to positions 114, 116, 120, 123, 70, 73, 75, 77 and 79 of SEQ ID NO: 2. If the or each monomer in the pore comprises a variant of SEQ ID NO: 2 in which only amino acids substitutions are made, the variant preferably comprises a positively charged amino acid at one or more of positions 114, 116, 120, 123, 70, 73, 75, 77 and 79. Positive charges at one or more of these positions neutralise the negative charge at position 118. Positively charged amino acids may be present at any number and combination of the positions which correspond to positions 114, 116, 120, 123, 70, 73, 75, 77 and 79 or at positions 114, 116, 120, 123, 70, 73, 75, 77 and 79. The amino acids may be introduced by addition or substitution.

Amino acids 134 and 139 in each monomer are part of the cap. The or each variant preferably comprises a positively charged amino acid at one or more of positions which correspond to positions 129, 132, 136, 137, 59, 61 and 63 in SEQ ID NO: 2. If the or each monomer in the pore comprises a variant of SEQ ID NO: 2 in which only amino acids substitutions are made, the or each variant preferably comprises a positively charged amino acid at one or more of positions 129, 132, 136, 137, 59, 61 and 63. Positive charges at one or more of these positions neutralise the negative charge at position 134. Positively charged amino acids may be present at any number and combination of the positions which correspond to positions 129, 132, 136, 137, 59, 61 and 63 in SEQ ID NO: 2 or at positions 129, 132, 136, 137, 59, 61 and 63. The amino acids may be introduced by addition or substitution.

The variant preferably comprises a positively charged amino acid at one or more of positions which correspond to positions 137, 138, 141, 143, 45, 47, 49 and 51 of SEQ ID NO: 2 or at positions 137, 138, 141, 143, 45, 47, 49 and 51. Positive charges at one or more of these positions neutralise the negative charge at position 139. Positively charged amino acids may be present at any number and combination of the positions which correspond to positions 137, 138, 141, 143, 45, 47, 49 and 51 of SEQ ID NO: 2 or at positions 137, 138, 141, 143, 45, 47, 49 and 51. The amino acids may be introduced by addition or substitution.

### Positions 118, 126, 134 and 139

The one or more modifications preferably reduce the net negative charge at one or more of the positions which correspond to positions 118, 126, 134 and 139 in SEQ ID NO: 2 or at positions 118, 126, 134 and 139. The one or more modifications preferably reduce the net negative charge at the position(s) which corresponds to position(s) or at position(s) 118; 126; 134; 139; 118 and 126; 118 and 134; 118 and 139; 126 and 134; 126 and 139; 134 and 139; 118, 126 and 134; 118, 126 and 139; 118, 134 and 139; 126, 134 and 139; or 118, 126, 134 and 139 in SEQ ID NO: 2.

The variant preferably does not comprise aspartic acid (D) or glutamic acid (E) at one or more of the positions which correspond to positions 118, 126, 134 and 139 of SEQ ID NO: 2 or at positions 118, 126, 134 and 139. The variant preferably does not comprise aspartic acid (D) or glutamic acid (E) at any of the combination of positions which correspond to positions 118, 126, 134 and 139 or at positions 118, 126, 134 and 139 disclosed above. The variant more preferably comprises arginine (R), glycine (G) or asparagine (N) at one or more of the positions which correspond to positions 118, 126, 134 and 139 of SEQ ID NO: 2 or at positions 118, 126, 134 and 139, such as any of the combinations of positions 118, 126, 134 and 139 disclosed above. The variant most preferably comprises D118R, Q126R, D134R and E139K.

Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. The polynucleotide can then be expressed as discussed above.

The one or more modifications are preferably one or more chemical modifications of one or more negatively charged amino acids which neutralise their negative charge. For instance, the one or more negatively charged amino acids may be reacted with a carbodiimide.

### Other Modifications

The variant preferably comprises one or more of:
(e) serine(S) at the position which corresponds to position 75 of SEQ ID NO: 2 or at position 75;
(f) serine(S) at the position which corresponds to position 77 of SEQ ID NO: 2 or at position 77; and
(g) asparagine (N) or lysine (K) at the position which corresponds to position 88 in SEQ ID NO: 2 or at position 88.

The variant may comprise any number and combination of (e) to (g), including (e), (f), (g), (e) and (f), (f) and (g), (e) and (g) and (e), (f) and (g). The variant preferably comprises G75S, G77S and L88N.

The variant most preferably comprises (a) D90N, D91N, D93N, D118R, D134R and E139K, (b) L88N, D90N, D91N, D93N, D118R, D134R and E139K, (c) G75S, G77S, L88N, D90N, D91N, D93N, D118R, Q126R, D134R and E139K or (d) G75S, G77S, L88N, D90N, D91N, D118R, Q126R, D134R and E139K. Where the positions in (a) to (d) correspond to those in SEQ ID NO: 2 or are the positions in the variant.

The variant preferably further comprises one or more of:
(i) phenylalanine (F) at the position which corresponds to position 89 of SEQ ID NO: 2 or at position 89;
(j) glutamic acid (E) at the position which corresponds to position 95 of SEQ ID NO: 2 or at position 95 and lysine (K) at the position which corresponds to position 98 of SEQ ID NO: 2 or at position 98;
(l) aspartic acid (D) at the position which corresponds to position 96 of SEQ ID NO: 2 or at position 96;
(m) glycine (G) at the position which corresponds to position 102 of SEQ ID NO: 2 or at position 102;
(n) alanine (A) at the position which corresponds to position 103 of SEQ ID NO: 2 or at position 103; and
(o) alanine (A), serine(S) or proline (P) at the position which corresponds to position 108 or at position 108.

The variant may comprise any number and combination of (i) to (o).

### Improved Movement

The or each variant preferably comprises one or more modifications in a part of the variant which interacts with a polynucleotide binding protein. This improves the movement of a target polynucleotide with respect to a pore comprising the variant when the movement is controlled by a polynucleotide binding protein. These modifications and their advantages are discussed in International Application No. PCT/GB2015/051291 (published as WO 2015/166276). The one or more modifications preferably provide more consistent movement of the target polynucleotide with respect to, such as through, a transmembrane pore comprising the variant. The one or more modifications preferably reduce the noise associated with the movement of the target polynucleotide with respect to, such as through, a transmembrane pore comprising the variant. If the target polynucleotide is double stranded, the one or more modifications preferably reduce the noise associated with movement of the complement strand relative to the template strand and/or provide more consistent movement of the complement strand relative to the template strand. This is advantageous for strand sequencing of double stranded target polynucleotides. The two stands of the double stranded polynucleotide are preferably linked by a bridging moiety, such as a hairpin loop or hairpin loop adaptor. This is discussed in more detail below.

Any number of modifications can be made, such as 2 or more, 3 or more, 5 or more, 10 or more, 15 or more, 20 or more, 25 or more, 30 or more, 50 or more or 100 or more modifications.

The part of the variant which interacts with the polynucleotide binding protein typically comprises the amino acids which correspond to positions 12, 14, 48, 52, 53, 54, 55, 56, 57, 58, 59, 60, 134, 135, 136, 137, 138, 139, 169 and 170 of SEQ ID NO: 2 or at positions 12, 14, 48, 52, 53, 54, 55, 56, 57, 58, 59, 60, 134, 135, 136, 137, 138, 139, 169 and 170.

The part of the variant which interacts with the polynucleotide binding protein preferably comprises the amino acids
(a) which correspond to positions 12, 14, 52, 54, 56, 57, 59, 134, 136, 138, 139 and 169 in SEQ ID NO: 2 or at positions 12, 14, 52, 54, 56, 57, 59, 134, 136, 138, 139 and 169;
(b) 12, 14, 56, 57, 59, 134, 136, 139 and 169 in SEQ ID NO: 2 or at positions 12, 14, 56, 57, 59, 134, 136, 139 and 169;
(c) 56, 57, 59, 134, 136, 139 and 169 in SEQ ID NO: 2 or at positions 56, 57, 59, 134, 136, 139 and 169; or
(d) 56, 57, 59, 134 and 139 in SEQ ID NO: 2 or at positions 56, 57, 59, 134 and 139.

Any modifications may be made in accordance with the invention. The variant may comprise one or more modifications which (a) alter the charge, (b) alter the sterics, (c) alter the hydrogen bonding, (d) alter the x stacking or (e) alter the structure of the part of the variant which interacts with the polynucleotide binding protein. Any number and combination of these may be altered. For instance, the method may involve making one of more modifications which {a}; {b}; {c}; {d}; {e}; {a,b}; {a,c}; {a,d}; {a,e}; {b,c}; {b,d}; {b,e}; {c,d}; {c,e}; {d,e}; {a,b,c}; {a,b,d}; {a,b,e}; {a,c,d}; {a,c,e}; {a,d,e}; {b,c,d}; {b,c,e}; {b,d,e}; {c,d,e}; {a,b,c,d}; {a,b,c,e}; {a,b,d,e}; {a,c,d,e}; {b,c,d,e}; or {a,b,c,d,e}.

When modifying the variant, the one or more modifications typically involve introducing or replacing one or more amino acids. The invention typically involves making one or more amino acid substitutions.

Modifications which alter the charge may involve increasing the net negative charge or decreasing the net negative charge. The method preferably comprises making one or more modifications which decrease the net negative charge of the part of the variant which interacts with the polynucleotide binding protein. Modifications which decrease net negative charge are discussed in more detail above. In a preferred embodiment, the variant does not comprise aspartic acid (D) or glutamic acid (E) at one or more of the positions which correspond to positions 56, 57, 59, 134 and 139 of SEQ ID NO: 2 or at positions 56, 57, 59, 134 and 139. The variant preferably comprises one or more of (a) D56N or D56R, (b) E57N or E57R, (c) E59N or E59R, (d) D134N or D134R and (e) E139N, E139R or E139K. The variant may comprise any number and combination of these modifications. For instance, one or more of the monomers may comprise {a}; {b}; {c}; {d}; {e}; {a,b}; {a,c}; {a,d}; {a,e}; {b,c}; {b,d}; {b,e}; {c,d}; {c,e}; {d,e}; {a,b,c}; {a,b,d}; {a,b,e}; {a,c,d}; {a,c,e}; {a,d,e}; {b,c,d}; {b,c,e}; {b,d,e}; {c,d,e}; {a,b,c,d}; {a,b,c,e}; {a,b,d,e}; {a,c,d,e}; {b,c,d,e}; or {a,b,c,d,e}.

Modifications which alter the sterics may involve increasing or decreasing the size of amino acid residues, for instance by substitution. For instance, sterics can be increased by the introduction of one or more bulky amino acids, such as phenylalanine (F), tryptophan (W), tyrosine (Y) and histidine (H).

Modifications which alter the hydrogen bonding may involve the introduction or replacement of one or more amino acids which can hydrogen bond.

Modifications which alter the x stacking may involve the introduction or replacement of amino acids that interact through delocalised electron x systems. For instance, α stacking can be increased by the introduction of one or more aromatic amino acids, such as phenylalanine (F), tryptophan (W), tyrosine (Y) and histidine (H).

### Variants

In addition to the specific mutations discussed above, the variant may include other mutations. Over the entire length of the amino acid sequence of SEQ ID NO: 2, a variant will preferably be at least 50% homologous to that sequence based on amino acid similarity or identity. More preferably, the variant may be at least 55%, at least 60%, at least 65%, at least 70%, at least 75%, at least 80%, at least 85%, at least 90% and more preferably at least 95%, 97% or 99% homologous based on amino acid similarity or identity to the amino acid sequence of SEQ ID NO: 2 over the entire sequence. There may be at least 80%, for example at least 85%, 90% or 95%, amino acid similarity or identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

Similarity can be measured using pairwise identity or by applying a scoring matrix such as BLOSUM62 and converting to an equivalent identity. Since they represent functional rather than evolved changes, deliberately mutated positions would be masked when determining homology. Similarity may be determined more sensitively by the application of position-specific scoring matrices using, for example, PSIBLAST on a comprehensive database of protein sequences. A different scoring matrix could be used that reflect amino acid chemico-physical properties rather than frequency of substitution over evolutionary time scales (e.g. charge).

SEQ ID NO: 2 is the mature form of the wild-type MspA monomer. The variant may comprise any of the mutations in the MspB, C or D monomers compared with MspA. The mature forms of MspB, C and D are shown in SEQ ID NOs: 5 to 7. In particular, the variant may comprise the following substitution present in MspB: A138P. The variant may comprise one or more of the following substitutions present in MspC: A96G, N102E and A138P. The variant may comprise one or more of the following mutations present in MspD: Deletion of G1, L2V, E5Q, L8V, D13G, W21A, D22E, K47T, I49H, I68V, D91G, A96Q, N102D, S103T, V104I, S136K and G141A. The variant may comprise combinations of one or more of the mutations and substitutions from Msp B, C and D.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 4 below. Where amino acids have similar polarity, this can also be determined by reference to the hydropathy scale for amino acid side chains in Table 5.

TABLE 4
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, |
| | | | charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, |
| | | | neutral |
| Lys | polar, hydrophilic, charged(+) | Trp | aromatic, hydrophobic, |
| | | | neutral |
| Leu | aliphatic, hydrophobic, neutral | Tyr | aromatic, polar, hydrophobic |

TABLE 5
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 2 may additionally be deleted from the polypeptides described above. Up to 1, 2, 3, 4, 5, 10, 20 or 30 residues may be deleted, or more.

Variants may include fragments of SEQ ID NO: 2. Such fragments retain pore forming activity. Fragments may be at least 50, 100, 150 or 200 amino acids in length. Such fragments may be used to produce the pores. A fragment preferably comprises the pore forming domain of SEQ ID NO: 2. Fragments must include one of residues which correspond to positions 88, 90, 91, 105, 118 and 134 of SEQ ID NO: 2. Typically, fragments include all of residues 88, 90, 91, 105, 118 and 134 of SEQ ID NO: 2.

One or more amino acids may be alternatively or additionally added to the polypeptides described above. An extension may be provided at the amino terminal or carboxy terminal of the amino acid sequence of SEQ ID NO: 2 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 2 and which retains its ability to form a pore. A variant typically contains the regions of SEQ ID NO: 2 that are responsible for pore formation. The pore forming ability of Msp, which contains a β-barrel, is provided by β-sheets in each subunit. A variant of SEQ ID NO: 2 typically comprises the regions in SEQ ID NO: 2 that form β-sheets. One or more modifications can be made to the regions of SEQ ID NO: 2 that form β-sheets as long as the resulting variant retains its ability to form a pore. A variant of SEQ ID NO: 2 preferably includes one or more modifications, such as substitutions, additions or deletions, within its α-helices and/or loop regions.

The monomers may be modified to assist their identification or purification, for example by the addition of a streptavidin tag or by the addition of a signal sequence to promote their secretion from a cell where the monomer does not naturally contain such a sequence. Other suitable tags are discussed in more detail below. The monomers may be labelled with a revealing label. The revealing label may be any suitable label which allows the monomers to be detected. Suitable labels are described below.

The monomers may also be produced using D-amino acids. For instance, the monomers may comprise a mixture of L-amino acids and D-amino acids. This is conventional in the art for producing such proteins or peptides.

The monomers typically contain one or more specific modifications to facilitate nucleotide discrimination. The monomers may also contain other non-specific modifications as long as they do not interfere with pore formation. A number of non-specific side chain modifications are known in the art and may be made to the side chains of the monomer derived from Msp. Such modifications include, for example, reductive alkylation of amino acids by reaction with an aldehyde followed by reduction with NaBH₄, amidination with methylacetimidate or acylation with acetic anhydride.

The monomers can be produced using standard methods known in the art. The monomer may be made synthetically or by recombinant means. For example, the monomers may be synthesized by in vitro translation and transcription (IVTT). Suitable methods for producing pores and monomers are discussed in International Application Nos. PCT/GB09/001690 (published as WO 2010/004273), PCT/GB09/001679 (published as WO 2010/004265) or PCT/GB10/000133 (published as WO 2010/086603). Methods for inserting pores into membranes are known in the art.

### Chemical Modification

In some embodiments, a monomer is chemically modified. The monomer is typically chemically modified once it has formed a hetero-oligomeric pore in accordance with the invention. The monomer can be chemically modified in any way and at any site. The monomer is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The monomer may be chemically modified by the attachment of any molecule. For instance, the monomer may be chemically modified by attachment of a dye or a fluorophore.

In other embodiment, the monomer may be attached to a polynucleotide binding protein. This forms a modular sequencing system that may be used in the methods of sequencing of the invention. Polynucleotide binding proteins are discussed below.

The polynucleotide binding protein is preferably covalently attached to the monomer. The protein can be covalently attached to the monomer using any method known in the art. The monomer and protein may be chemically fused or genetically fused. The monomer and protein are genetically fused if the whole construct is expressed from a single polynucleotide sequence. Genetic fusion of a monomer to a polynucleotide binding protein is discussed in International Application No. PCT/GB09/001679 (published as WO 2010/004265).

If the polynucleotide binding protein is attached via cysteine linkage, the one or more cysteines have preferably been introduced to the monomer by substitution. The monomer may comprise cysteine residues at one or more of the positions which correspond to positions 10 to 15, 51 to 60, 136 to 139 and 168 to 172 of SEQ ID NO: 2 or at positions 10 to 15, 51 to 60, 136 to 139 and 168 to 172. These positions are present in loop regions which have low conservation amongst homologues indicating that mutations or insertions may be tolerated. They are therefore suitable for attaching a polynucleotide binding protein. The reactivity of cysteine residues may be enhanced by modification as described above.

The polynucleotide binding protein may be attached directly to the monomer or via one or more linkers. The molecule may be attached to the monomer using the hybridization linkers described in International Application No. PCT/GB 10/000132 (published as WO 2010/086602). Alternatively, peptide linkers may be used. Peptide linkers are amino acid sequences. The length, flexibility and hydrophilicity of the peptide linker are typically designed such that it does not to disturb the functions of the monomer and molecule. Preferred flexible peptide linkers are stretches of 2 to 20, such as 4, 6, 8, 10 or 16, serine and/or glycine amino acids. More preferred flexible linkers include (SG) 1, (SG) 2, (SG) 3, (SG) 4, (SG) 5 and (SG) 8 wherein S is serine and G is glycine. Preferred rigid linkers are stretches of 2 to 30, such as 4, 6, 8, 16 or 24, proline amino acids. More preferred rigid linkers include (P) 12 wherein P is proline.

The monomer may be chemically modified with a molecular adaptor and a polynucleotide binding protein.

The reactivity of cysteine residues may be enhanced by modification of the adjacent residues. For instance, the basic groups of flanking arginine, histidine or lysine residues will change the pKa of the cysteines thiol group to that of the more reactive S group. The reactivity of cysteine residues may be protected by thiol protective groups such as dTNB. These may be reacted with one or more cysteine residues of the monomer before a linker is attached.

The molecule (with which the monomer is chemically modified) may be attached directly to the monomer or attached via a linker as disclosed in International Application Nos. PCT/GB09/001690 (published as WO 2010/004273), PCT/GB09/001679 (published as WO 2010/004265) or PCT/GB10/000133 (published as WO 2010/086603).

Any of the proteins described herein, such as the monomers and pores of the invention, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the protein. An example of this would be to react a gel-shift reagent to a cysteine engineered on the outside of the protein. This has been demonstrated as a method for separating hemolysin hetero-oligomers (Chem Biol. 1997 July; 4(7): 497-505).

Any of the proteins described herein, such as the monomers and pores of the invention, may be labelled with a revealing label. The revealing label may be any suitable label which allows the protein to be detected. Suitable labels include, but are not limited to, fluorescent molecules, radioisotopes, e.g. ¹²⁵I, ³⁵S, enzymes, antibodies, antigens, polynucleotides and ligands such as biotin.

Any of the proteins described herein, such as the monomers or pores of the invention, may be made synthetically or by recombinant means. For example, the protein may be synthesized by in vitro translation and transcription (IVTT). The amino acid sequence of the protein may be modified to include non-naturally occurring amino acids or to increase the stability of the protein. When a protein is produced by synthetic means, such amino acids may be introduced during production. The protein may also be altered following either synthetic or recombinant production.

Proteins may also be produced using D-amino acids. For instance, the protein may comprise a mixture of L-amino acids and D-amino acids. This is conventional in the art for producing such proteins or peptides.

The protein may also contain other non-specific modifications as long as they do not interfere with the function of the protein. A number of non-specific side chain modifications are known in the art and may be made to the side chains of the protein(s). Such modifications include, for example, reductive alkylation of amino acids by reaction with an aldehyde followed by reduction with NaBH₄, amidination with methylacetimidate or acylation with acetic anhydride.

Any of the proteins described herein, including the monomers and pores of the invention, can be produced using the method of the invention or standard methods known in the art. Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

### Constructs

The hetero-oligomeric pore of the invention may comprise a first different construct comprising two or more covalently attached monomers and a second different construct comprising two or more covalently attached monomers. Any of the construct embodiments discussed above apply to the pore of the invention.

### Polynucleotide Characterisation

The invention provides a method of characterising a target polynucleotide. The method involves measuring one or more characteristics of the target polynucleotide. The target polynucleotide may also be called the template polynucleotide or the polynucleotide of interest.

### Polynucleotide

A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag. Suitable labels are described below. The polynucleotide may comprise one or more spacers.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose.

The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The polynucleotide can comprise one strand of RNA hybridised to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety.

The polynucleotide is most preferably ribonucleic nucleic acid (RNA) or deoxyribonucleic acid (DNA).

The polynucleotide can be any length. For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length.

Any number of polynucleotides can be investigated. For instance, the method of the invention may concern characterising 2, 3, 4, 5, 6, 7, 8, 9, 10, 20, 30, 50, 100 or more polynucleotides. If two or more polynucleotides are characterized, they may be different polynucleotides or two instances of the same polynucleotide.

The polynucleotide can be naturally occurring or artificial. For instance, the method may be used to verify the sequence of a manufactured oligonucleotide. The method is typically carried out in vitro.

### Sample

Each analyte is typically present in any suitable sample. The invention is typically carried out on two or more samples that are known to contain or suspected to contain the analytes. Alternatively, the invention may be carried out on two or more samples to confirm the identity of two or more analytes whose presence in the samples is known or expected.

The sample may be a biological sample. The invention may be carried out in vitro using at least one sample obtained from or extracted from any organism or microorganism. The organism or microorganism is typically archaeal, prokaryotic or eukaryotic and typically belongs to one of the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep, fish, chickens or pigs or may alternatively be pets such as cats or dogs. Alternatively, the first sample and/or second sample may be of plant origin, such as a sample obtained from a commercial crop, such as a cereal, legume, fruit or vegetable, for example wheat, barley, oats, canola, maize, soya, rice, rhubarb, bananas, apples, tomatoes, potatoes, grapes, tobacco, beans, lentils, sugar cane, cocoa, cotton.

The sample may be a non-biological sample. The non-biological sample is preferably a fluid sample. Examples of non-biological samples include surgical fluids, water such as drinking water, sea water or river water, and reagents for laboratory tests.

The sample is typically processed prior to being used in the invention, for example by centrifugation or by passage through a membrane that filters out unwanted molecules or cells, such as red blood cells. The sample may be measured immediately upon being taken. The sample may also be typically stored prior to assay, preferably below −70° C.

### Characterisation

The method may involve measuring two, three, four or five or more characteristics of the polynucleotide. The one or more characteristics are preferably selected from (i) the length of the polynucleotide, (ii) the identity of the polynucleotide, (iii) the sequence of the polynucleotide, (iv) the secondary structure of the polynucleotide and (v) whether or not the polynucleotide is modified. Any combination of (i) to (v) may be measured in accordance with the invention, such as {i}, {ii}, {iii}, {iv}, {v}, {i,ii}, {i,iii}, {i,iv}, {i,v}, {ii, iii}, {ii,iv}, {ii,v}, {iii,iv}, {iii,v}, {iv,v}, {i,ii,iii}, {i,ii,iv}, {i,ii,v}, {i,iii,iv}, {i,iii,v}, {i,iv,v}, {ii,iii,iv}, {ii,iii,v}, {ii,iv,v}, {iii,iv,v}, {i,ii,iii,iv}, {i,ii,iii,v}, {i,ii,iv,v}, {i,iii,iv,v}, {ii,iii,iv,v} or {i,ii,iii,iv,v}. Different combinations of (i) to (v) may be measured for the first polynucleotide compared with the second polynucleotide, including any of those combinations listed above.

For (i), the length of the polynucleotide may be measured for example by determining the number of interactions between the polynucleotide and the pore or the duration of interaction between the polynucleotide and the pore.

For (ii), the identity of the polynucleotide may be measured in a number of ways. The identity of the polynucleotide may be measured in conjunction with measurement of the sequence of the polynucleotide or without measurement of the sequence of the polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively, the measurement of a particular electrical and/or optical signal in the method may identify the polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12; 106 (19): 7702-7, Lieberman K R et al, J Am Chem Soc. 2010; 132 (50): 17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the polynucleotide is modified by methylation, by oxidation, by damage, with one or more proteins or with one or more labels, tags or spacers. Specific modifications will result in specific interactions with the pore which can be measured using the methods described below. For instance, methylcytosine may be distinguished from cytosine on the basis of the current flowing through the pore during its interaction with each nucleotide.

The target polynucleotide is contacted with a hetero-oligomeric pore of the invention. The pore is typically present in a membrane. Suitable membranes are discussed below. The method may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane in which the pore is present.

The method may be carried out using the apparatus described in International Application No. PCT/GB08/000562 (WO 2008/102120).

A variety of different types of measurements may be made. This includes without limitation: electrical measurements and optical measurements. Possible electrical measurements include: current measurements, impedance measurements, tunnelling measurements (Ivanov A P et al., Nano Lett. 2011 Jan. 12; 11 (1): 279-85), and FET measurements (International Application WO 2005/124888). Optical measurements may be combined with electrical measurements (Soni G V et al., Rev Sci Instrum. 2010 January; 81 (1): 014301). The measurement may be a transmembrane current measurement such as measurement of ionic current flowing through the pore.

Electrical measurements may be made using standard single channel recording equipment as described in Stoddart D et al., Proc Natl Acad Sci, 12; 106 (19): 7702-7, Lieberman K R et al, J Am Chem Soc. 2010; 132 (50): 17961-72, and International Application WO 2000/28312. Alternatively, electrical measurements may be made using a multi-channel system, for example as described in International Application WO 2009/077734 and

### International Application WO 2011/067559.

The method is preferably carried out with a potential applied across the membrane. The applied potential may be a voltage potential. Alternatively, the applied potential may be a chemical potential. An example of this is using a salt gradient across a membrane, such as an amphiphilic layer. A salt gradient is disclosed in Holden et al., J Am Chem Soc. 2007 Jul. 11; 129 (27): 8650-5. In some instances, the current passing through the pore as a polynucleotide moves with respect to the pore is used to estimate or determine the sequence of the polynucleotide. This is strand sequencing.

The method may involve measuring the current passing through the pore as the polynucleotide moves with respect to the pore. Therefore the apparatus used in the method may also comprise an electrical circuit capable of applying a potential and measuring an electrical signal across the membrane and pore. The methods may be carried out using a patch clamp or a voltage clamp. The methods preferably involve the use of a voltage clamp.

The method of the invention may involve the measuring of a current passing through the pore as the polynucleotide moves with respect to the pore. Suitable conditions for measuring ionic currents through transmembrane protein pores are known in the art and disclosed in the Example. The method is typically carried out with a voltage applied across the membrane and pore. The voltage used is typically from +5 V to −5 V, such as from +4 V to −4 V, +3 V to −3 V or +2 V to −2 V. The voltage used is typically from −600 mV to +600 mV or −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The method is typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The method is typically carried out in the presence of a buffer. In the exemplary apparatus discussed above, the buffer is present in the aqueous solution in the chamber. Any buffer may be used in the method of the invention. Typically, the buffer is phosphate buffer. Other suitable buffers are HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The method may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

### Polynucleotide Binding Protein

Step (a) preferably comprises contacting the polynucleotide with a polynucleotide binding protein such that the protein controls the movement of the polynucleotide through the pore.

More preferably, the method comprises (a) contacting the polynucleotide with the pore of the invention and a polynucleotide binding protein such that the protein controls the movement of the polynucleotide through the pore and (b) taking one or more measurements as the polynucleotide moves with respect to the pore, wherein the measurements are indicative of one or more characteristics of the polynucleotide, and thereby characterising the polynucleotide.

The polynucleotide binding protein may be any protein that is capable of binding to the polynucleotide and controlling its movement through the pore. It is straightforward in the art to determine whether or not a protein binds to a polynucleotide. The protein typically interacts with and modifies at least one property of the polynucleotide. The protein may modify the polynucleotide by cleaving it to form individual nucleotides or shorter chains of nucleotides, such as di- or trinucleotides. The protein may modify the polynucleotide by orienting it or moving it to a specific position, i.e. controlling its movement.

The polynucleotide binding protein is preferably derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide handling enzyme does not need to display enzymatic activity as long as it is capable of binding the polynucleotide and controlling its movement through the pore. For instance, the enzyme may be modified to remove its enzymatic activity or may be used under conditions which prevent it from acting as an enzyme. Such conditions are discussed in more detail below.

The polynucleotide handling enzyme is preferably derived from a nucleolytic enzyme. The polynucleotide handling enzyme used in the construct of the enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in International Application No. PCT/GB10/000133 (published as WO 2010/086603).

Preferred enzymes are polymerases, exonucleases, helicases and topoisomerases, such as gyrases. Suitable enzymes include, but are not limited to, exonuclease I from E. coli (SEQ ID NO: 11), exonuclease III enzyme from E. coli (SEQ ID NO: 13), RecJ from T. thermophilus (SEQ ID NO: 15) and bacteriophage lambda exonuclease (SEQ ID NO: 17), TatD exonuclease and variants thereof. Three subunits comprising the sequence shown in SEQ ID NO: 15 or a variant thereof interact to form a trimer exonuclease. The polymerase may be PyroPhage® 3173 DNA Polymerase (which is commercially available from Lucigen® Corporation), SD Polymerase (commercially available from Bioron®) or variants thereof. The enzyme is preferably Phi29 DNA polymerase (SEQ ID NO: 9) or a variant thereof. The topoisomerase is preferably a member of any of the Moiety Classification (EC) groups 5.99.1.2 and 5.99.1.3.

The enzyme is most preferably derived from a helicase, such as Hel308 Mbu (SEQ ID NO: 18), Hel308 Csy (SEQ ID NO: 19), Hel308 (Tga), Hel308 Mhu (SEQ ID NO: 21), TraI Eco (SEQ ID NO: 22), XPD Mbu (SEQ ID NO: 23) or a variant thereof. Any helicase may be used in the invention. The helicase may be or be derived from a Hel308 helicase, a RecD helicase, such as TraI helicase or a TrwC helicase, a XPD helicase or a Dda helicase. The helicase may be any of the helicases, modified helicases or helicase constructs disclosed in International Application Nos. PCT/GB2012/052579 (published as WO 2013/057495); PCT/GB2012/053274 (published as WO 2013/098562); PCT/GB2012/053273 (published as WO2013098561); PCT/GB2013/051925 (published as WO 2014/013260); PCT/GB2013/051924 (published as WO 2014/013259); PCT/GB2013/051928 (published as WO 2014/013262); PCT/GB2014/052736 (published as WO 2015/055981) and PCT/GB2015/052916.

The helicase preferably comprises the sequence shown in SEQ ID NO: 25 (Trwc Cba) or a variant thereof, the sequence shown in SEQ ID NO: 18 (Hel308 Mbu) or a variant thereof or the sequence shown in SEQ ID NO: 24 (Dda) or a variant thereof. Variants may differ from the native sequences in any of the ways discussed below for transmembrane pores. A preferred variant of SEQ ID NO: 24 comprises (a) E94C and A360C or (b) E94C, A360C, C109A and C136A and then optionally (AM1) G1G2 (i.e. deletion of M1 and then addition G1 and G2).

Any number of helicases may be used in accordance with the invention. For instance, 1, 2, 3, 4, 5, 6, 7, 8, 9, 10 or more helicases may be used. In some embodiments, different numbers of helicases may be used.

The method of the invention preferably comprises contacting the polynucleotide with two or more helicases. The two or more helicases are typically the same helicase. The two or more helicases may be different helicases.

The two or more helicases may be any combination of the helicases mentioned above. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. Preferred helicase constructs for use in the invention are described in International Application Nos. PCT/GB2013/051925 (published as WO 2014/013260); PCT/GB2013/051924 (published as WO 2014/013259); PCT/GB2013/051928 (published as WO 2014/013262); PCT/GB2014/052736 (published as WO 2015/055981) and PCT/GB2015/052916.

A variant of SEQ ID NOs: 9, 11, 13, 15, 17, 18, 19, 20, 21, 22, 23, 24 or 25 is an enzyme that has an amino acid sequence which varies from that of SEQ ID NO: 9, 11, 13, 15, 17, 18, 19, 20, 21, 22, 23, 24 or 25 and which retains polynucleotide binding ability. This can be measured using any method known in the art. For instance, the variant can be contacted with a polynucleotide and its ability to bind to and move along the polynucleotide can be measured. The variant may include modifications that facilitate binding of the polynucleotide and/or facilitate its activity at high salt concentrations and/or room temperature. Variants may be modified such that they bind polynucleotides (i.e. retain polynucleotide binding ability) but do not function as a helicase (i.e. do not move along polynucleotides when provided with all the necessary components to facilitate movement, e.g. ATP and Mg²⁺). Such modifications are known in the art. For instance, modification of the Mg²⁺ binding domain in helicases typically results in variants which do not function as helicases. These types of variants may act as molecular brakes (see below).

Over the entire length of the amino acid sequence of SEQ ID NO: 9, 11, 13, 15, 17, 18, 19, 20, 21, 22, 23, 24 or 25, a variant will preferably be at least 50% homologous to that sequence based on amino acid similarity or identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid similarity or identity to the amino acid sequence of SEQ ID NO: 9, 11, 13, 15, 17, 18, 19, 20, 21, 22, 23, 24 or 25 over the entire sequence. There may be at least 80%, for example at least 85%, 90% or 95%, amino acid similarity or identity over a stretch of 200 or more, for example 230, 250, 270, 280, 300, 400, 500, 600, 700, 800, 900 or 1000 or more, contiguous amino acids (“hard homology”). Homology is determined as described above. The variant may differ from the wild-type sequence in any of the ways discussed above with reference to SEQ ID NO: 2 and 4 above. The enzyme may be covalently attached to the pore. Any method may be used to covalently attach the enzyme to the pore.

A preferred molecular brake is TrwC Cba-Q594A (SEQ ID NO: 25 with the mutation Q594A). This variant does not function as a helicase (i.e. binds polynucleotides but does not move along them when provided with all the necessary components to facilitate movement, e.g. ATP and Mg²⁺).

In strand sequencing, the polynucleotide is translocated through the pore either with or against an applied potential. Exonucleases that act progressively or processively on double stranded polynucleotides can be used on the cis side of the pore to feed the remaining single strand through under an applied potential or the trans side under a reverse potential. Likewise, a helicase that unwinds the double stranded DNA can also be used in a similar manner. A polymerase may also be used. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

Any helicase may be used in the method. Helicases may work in two modes with respect to the pore. First, the method is preferably carried out using a helicase such that it moves the polynucleotide through the pore with the field resulting from the applied voltage. In this mode the 5′ end of the polynucleotide is first captured in the pore, and the helicase moves the polynucleotide into the pore such that it is passed through the pore with the field until it finally translocates through to the trans side of the membrane. Alternatively, the method is preferably carried out such that a helicase moves the polynucleotide through the pore against the field resulting from the applied voltage. In this mode the 3′ end of the polynucleotide is first captured in the pore, and the helicase moves the polynucleotide through the pore such that it is pulled out of the pore against the applied field until finally ejected back to the cis side of the membrane.

The method may also be carried out in the opposite direction. The 3′ end of the polynucleotide may be first captured in the pore and the helicase may move the polynucleotide into the pore such that it is passed through the pore with the field until it finally translocates through to the trans side of the membrane.

When the helicase is not provided with the necessary components to facilitate movement or is modified to hinder or prevent its movement, it can bind to the polynucleotide and act as a brake slowing the movement of the polynucleotide when it is pulled into the pore by the applied field. In the inactive mode, it does not matter whether the polynucleotide is captured either 3′ or 5′ down, it is the applied field which pulls the polynucleotide into the pore towards the trans side with the enzyme acting as a brake. When in the inactive mode, the movement control of the polynucleotide by the helicase can be described in a number of ways including ratcheting, sliding and braking. Helicase variants which lack helicase activity can also be used in this way.

The polynucleotide may be contacted with the polynucleotide binding protein and the pore in any order. It is preferred that, when the polynucleotide is contacted with the polynucleotide binding protein, such as a helicase, and the pore, the polynucleotide firstly forms a complex with the protein. When the voltage is applied across the pore, the polynucleotide/protein complex then forms a complex with the pore and controls the movement of the polynucleotide through the pore.

Any steps in the method using a polynucleotide binding protein are typically carried out in the presence of free nucleotides or free nucleotide analogues and an enzyme cofactor that facilitates the action of the polynucleotide binding protein. The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (CAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the construct to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

### Helicase(s) and Molecular Brake(s)

In a preferred embodiment, the method comprises:
(a) providing the polynucleotide with one or more helicases and one or more molecular brakes attached to the polynucleotide;
(b) contacting the polynucleotide with a pore of the invention and applying a potential across the pore such that the one or more helicases and the one or more molecular brakes are brought together and both control the movement of the polynucleotide through the pore;
(c) taking one or more measurements as the polynucleotide moves with respect to the pore wherein the measurements are indicative of one or more characteristics of the polynucleotide and thereby characterising the polynucleotide.

This type of method is discussed in detail in the International Application PCT/GB2014/052737 (published as WO2015/110777).

The one or more helicases may be any of those discussed above. The one or more molecular brakes may be any compound or molecule which binds to the polynucleotide and slows the movement of the polynucleotide through the pore. The one or more molecular brakes preferably comprise one or more compounds which bind to the polynucleotide. The one or more compounds are preferably one or more macrocycles. Suitable macrocycles include, but are not limited to, cyclodextrins, calixarenes, cyclic peptides, crown ethers, cucurbiturils, pillararenes, derivatives thereof or a combination thereof. The cyclodextrin or derivative thereof may be any of those disclosed in Eliseev, A. V., and Schneider, H-J. (1994) J. Am. Chem. Soc. 116, 6081-6088. The agent is more preferably heptakis-6-amino-β-cyclodextrin (am₇-βCD), 6-monodeoxy-6-monoamino-β-cyclodextrin (am₁-βCD) or heptakis-(6-deoxy-6-guanidino)-cyclodextrin (gu₇-βCD).

The one or more molecular brakes are preferably one or more single stranded binding proteins (SSB). The one or more molecular brakes are more preferably a single-stranded binding protein (SSB) comprising a carboxy-terminal (C-terminal) region which does not have a net negative charge or (ii) a modified SSB comprising one or more modifications in its C-terminal region which decreases the net negative charge of the C-terminal region. The one or more molecular brakes are most preferably any of the SSBs disclosed in International Application No. PCT/GB2013/051924 (published as WO 2014/013259).

The one or more molecular brakes are preferably one or more polynucleotide binding proteins. The polynucleotide binding protein may be any protein that is capable of binding to the polynucleotide and controlling its movement through the pore. It is straightforward in the art to determine whether or not a protein binds to a polynucleotide. The protein typically interacts with and modifies at least one property of the polynucleotide. The protein may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

The polynucleotide binding protein is preferably derived from a polynucleotide handling enzyme. The one or more molecular brakes may be derived from any of the polynucleotide handling enzymes discussed above. Modified versions of Phi29 polymerase (SEQ ID NO: 8) which act as molecular brakes are disclosed in U.S. Pat. No. 5,576,204. The one or more molecular brakes are preferably derived from a helicase.

Any number of molecular brakes derived from a helicase may be used. For instance, 1, 2, 3, 4, 5, 6, 7, 8, 9, 10 or more helicases may be used as molecular brakes. If two or more helicases are used as molecular brakes, the two or more helicases are typically the same helicase. The two or more helicases may be different helicases.

The two or more helicases may be any combination of the helicases mentioned above. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. The one or more molecular brakes derived from helicases are preferably modified to reduce the size of an opening in the polynucleotide binding domain through which in at least one conformational state the polynucleotide can unbind from the helicase. This is disclosed in WO 2014/013260.

Preferred helicase constructs for use in the invention are described in International Application Nos. PCT/GB2013/051925 (published as WO 2014/013260); PCT/GB2013/051924 (published as WO 2014/013259); PCT/GB2013/051928 (published as WO 2014/013262) and PCT/GB2014/052736 (published as WO 2015/055981).

### Spacers

The one or more helicases may be stalled at one or more spacers as discussed in International Application No. PCT/GB2014/050175 (published as WO 2014/135838). Any configuration of one or more helicases and one or more spacers disclosed in the International Application may be used in this invention.

### Membrane

The pore of the invention may be present in a membrane. In the method of the invention, the polynucleotide is typically contacted with the pore of the invention in a membrane. Any membrane may be used in accordance with the invention. Suitable membranes are well-known in the art. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesized, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customize polymer based membranes for a wide range of applications.

The membrane is most preferably one of the membranes disclosed in International Application No. PCT/GB2013/052766 (published as WO 2014/064443) or PCT/GB2013/052767 (published as WO 2014/064444).

The amphiphilic molecules may be chemically-modified or functionalised to facilitate coupling of the polynucleotide.

The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

### Coupling

The polynucleotide is preferably coupled to the membrane comprising the pore of the invention. The method may comprise coupling the polynucleotide to the membrane comprising the pore of the invention. The polynucleotide is preferably coupled to the membrane using one or more anchors. The polynucleotide may be coupled to the membrane using any known method. Suitable coupling methods are disclosed in International Application No. PCT/GB2012/051191 (published as WO 2012/164270) and PCT/GB2015/050991 (published as WO 2015/150786).

### Double Stranded Polynucleotide

The polynucleotide may be double stranded. If the polynucleotide is double stranded, the method preferably further comprises before the contacting step ligating a bridging moiety, such as a hairpin loop, to one end of the polynucleotide. The two strands of the polynucleotide may then be separated as or before the polynucleotide is contacted with the pore in accordance with the invention. The two strands may be separated as the polynucleotide movement through the pore is controlled by a polynucleotide binding protein, such as a helicase, or molecular brake.

Linking and interrogating both strands on a double stranded construct in this way increases the efficiency and accuracy of characterisation as described in PCT/GB2010/000160 (published as WO 2010/086622) and PCT/GB2012/051786 (published as WO 2013/014451).

### Modified Polynucleotides

Before characterisation, a target polynucleotide may be modified by contacting the polynucleotide with a polymerase and a population of free nucleotides under conditions in which the polymerase forms a modified polynucleotide using the target polynucleotide as a template, wherein the polymerase replaces one or more of the nucleotide species in the target polynucleotide with a different nucleotide species when forming the modified polynucleotide. The modified polynucleotide may then be provided with one or more helicases attached to the polynucleotide and one or more molecular brakes attached to the polynucleotide. This type of modification is described in International Application No. PCT/GB2015/050483 (published as WO2015/124935). Any of the polymerases discussed above may be used. The polymerase is preferably Klenow or 9° North.

### Other Characterisation Method

In another embodiment, a polynucleotide is characterised by detecting labelled species that are released as a polymerase incorporates nucleotides into the polynucleotide. The polymerase uses the polynucleotide as a template. Each labelled species is specific for each nucleotide. The polynucleotide is contacted with a pore of the invention, a polymerase and labelled nucleotides such that phosphate labelled species are sequentially released when nucleotides are added to the polynucleotide(s) by the polymerase, wherein the phosphate species contain a label specific for each nucleotide. The polymerase may be any of those discussed above. The phosphate labelled species are detected using the pore and thereby characterising the polynucleotide. This type of method is disclosed in European Application No. 13187149.3 (published as EP 2682460). Any of the embodiments discussed above equally apply to this method.

### Kits

The present invention also provides a kit for characterising a target polynucleotide. The kit comprises a hetero-oligomeric pore of the invention and the components of a membrane. The membrane is preferably formed from the components. The pore is preferably present in the membrane. The kit may comprise components of any of the membranes disclosed above, such as an amphiphilic layer or a triblock copolymer membrane.

The kit may further comprise a polynucleotide binding protein.

The kit may further comprise one or more anchors for coupling the polynucleotide to the membrane.

The kit is preferably for characterising a double stranded polynucleotide and preferably comprises a Y adaptor and a bridging moiety adaptor, such as a hairpin loop adaptor. The Y adaptor preferably has one or more helicases attached and the bridging moiety adaptor or hairpin loop adaptor preferably has one or more molecular brakes attached. The Y adaptor preferably comprises one or more first anchors for coupling the polynucleotide to the membrane, the bridging moiety adaptor or hairpin loop adaptor preferably comprises one or more second anchors for coupling the polynucleotide to the membrane and the strength of coupling of the bridging moiety adaptor or hairpin loop adaptor to the membrane is preferably greater than the strength of coupling of the Y adaptor to the membrane.

The kit of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used.

### Apparatus

The invention also provides an apparatus for characterising a target polynucleotide. The apparatus comprises a plurality of hetero-oligomeric pores of the invention and a plurality of membranes. The plurality of pores are preferably present in the plurality of membranes. The number of pores and membranes is preferably equal. Preferably, a single pore is present in each membrane.

The apparatus preferably further comprises instructions for carrying out the method of the invention. The apparatus may be any conventional apparatus for polynucleotide analysis, such as an array or a chip. Any of the embodiments discussed above with reference to the methods of the invention are equally applicable to the apparatus of the invention. The apparatus may further comprise any of the features present in the kit of the invention.

The apparatus is preferably set up to carry out the method of the invention.

The apparatus preferably comprises:
a sensor device that is capable of supporting the plurality of pores and membranes and being operable to perform polynucleotide characterisation using the pores and membranes; and
at least one port for delivery of the material for performing the characterisation.

Alternatively, the apparatus preferably comprises:
a sensor device that is capable of supporting the plurality of pores and membranes being operable to perform polynucleotide characterisation using the pores and membranes; and
at least one reservoir for holding material for performing the characterisation.

The apparatus more preferably comprises:
a sensor device that is capable of supporting the membrane and plurality of pores and membranes and being operable to perform polynucleotide characterising using the pores and membranes;
at least one reservoir for holding material for performing the characterising;
a fluidics system configured to controllably supply material from the at least one reservoir to the sensor device; and
one or more containers for receiving respective samples, the fluidics system being configured to supply the samples selectively from one or more containers to the sensor device.

The apparatus may be any of those described in International Application No. PCT/GB08/004127 (published as WO 2009/077734), PCT/GB10/000789 (published as WO 2010/122293), International Application No. PCT/GB10/002206 (published as WO 2011/067559) or International Application No. PCT/US99/25679 (published as WO 00/28312).

### Methods of Forming Sensors

The invention also provides a method of forming a sensor for characterising a target polynucleotide. The method comprises forming a complex between a hetero-oligomeric pore of the invention and a polynucleotide binding protein, such as a helicase or an exonuclease. The complex may be formed by contacting the pore and the protein in the presence of the target polynucleotide and then applying a potential across the pore. The applied potential may be a chemical potential or a voltage potential as described above. Alternatively, the complex may be formed by covalently attaching the pore to the protein. Methods for covalent attachment are known in the art and disclosed, for example, in International Application Nos. PCT/GB09/001679 (published as WO 2010/004265) and PCT/GB10/000133 (published as WO 2010/086603). The complex is a sensor for characterising the target polynucleotide. The method preferably comprises forming a complex between a hetero-oligomeric pore of the invention and a helicase. Any of the embodiments discussed above equally apply to this method.

The invention also provides a sensor for characterising a target polynucleotide. The sensor comprises a complex between a hetero-oligomeric pore of the invention and a polynucleotide binding protein. Any of the embodiments discussed above equally apply to the sensor of the invention.

The following Example illustrates the invention.

### Example 1

This example describes a scaled up E. coli purification method that was used to purify an MspA hetero-oligomeric pore. The production of the mutant hetero-oligomeric nanopore MspA 1=MspA—((Del-L74/G75/D118/L119) D56N/E59R/L88N/D90N/D91N/Q126R/D134R/E139K) 7 ((Del-L74/G75/D118/L119) D56N/E59R/L88N/D91N/Q126R/D134R/E139K/BasTL) 1 (SEQ ID NO: 2 with the following mutations in seven monomers D56N/E59R/L88N/D90N/D91N/Q126R/D134R/E139K and deletion, of the following amino acids in seven monomers L74/G75/D118/L119 and the following mutations in one monomer of D56N/E59R/L88N/D91N/Q126R/D134R/E139K/BasTL, where the BasTL has SEQ ID NO: 26 and is attached at the C-terminus, and deletions of the following amino acids in one monomer of L74/G75/D118/L119) is described below. This method is suitable for making other hetero-oligomeric nanopores.

DNA encoding the polypeptide (SEQ ID NO: 27) for MspA—((Del-L74/G75/D118/L119) D56N/E59R/L88N/D90N/D91N/Q126R/D134R/E139K) (SEQ ID NO: 2 with the following mutations in the monomer D56N/E59R/L88N/D90N/D91N/Q126R/D134R/E139K and deletion, of the following amino acids in the monomers L74/G75/D118/L119) was synthesised (GenScript USA Inc.) and cloned into a pRham vector containing kanamycin resistance gene. Protein expression of the pRham vector can be induced by rhamnose. DNA encoding the polypeptide (SEQ ID NO: 28) MspA—(Del-L74/G75/D118/L119) D56N/E59R/L88N/D91N/Q126R/D134R/E139K/BasTL/H6) (SEQ ID NO: 2 with the following mutations in the monomer of D56N/E59R/L88N/D91N/Q126R/D134R/E139K/BasTL, where the BasTL has SEQ ID NO: 26 and is attached at the C-terminus, the six histidines (H6) are attached at the 3′ end of the BasTL at the C-terminus and deletions of the following amino acids in one monomer of L74/G75/D118/L119) was synthesised (GenScript USA Inc) and cloned into a pT7 vector containing ampicillin resistance gene. Protein expression of the pT7 vector can be induced by IPTG (Isopropyl β-D-thiogalactopyranoside). Concentrations of both DNA solutions were adjusted to 400 ng/uL. The two DNA solutions were then mixed together in a 1:1 ratio and 1 μL of the mixture was used to transform Lemo21 (DE3) competent E. coli cells (50 μl, NEB, catalogue number C2528H). The transformed cells were then incubated with SOC media (100 μl) with agitation at 37° C. for 2 hours. Cells were then plated out on LB agar containing both ampicillin (0.1 mg/ml) and kanamycin (0.034 mg/ml) and incubated for approx 16 hours at 37° C.

Bacterial colonies which grew in LB plates containing both ampicillin and kanamycin had been transformed with both plasmids: PRham containing the DNA of MspA—((Del-L74/G75/D118/L119) D56N/E59R/L88N/D90N/D91N/Q126R/D134R/E139K) and the pT7 containing the DNA of MspA—(Del-L74/G75/D118/L119) D56N/E59R/L88N/D91N/Q126R/D134R/E139K/BasTL/H6). One such colony was used to inoculate a starter culture of Terrific Broth (TB) media (10 mL) containing both ampicillin (0.1 mg/ml) and kanamycin (0.034 mg/ml). The starter culture was grown at 37° C. with agitation until OD600 was reached to 0.8-1.0. 1 mL of the starter culture was then used to inoculate 500 mL of TB media containing both ampicillin (0.1 mg/ml) and kanamycin (0.034 mg/ml). The culture was grown at 37° C. with agitation until OD600 was reached to 0.8-1.0. The temperature of the culture was then adjusted to 18° C. and induction was initiated by the addition of both IPTG (0.5 mM final concentration) and rhamnose (1.5% final concentration). Induction was carried out for approximately 18 hours with agitation at 18° C.

Following induction, the culture was pelleted by centrifugation at 6,000 g for 30 minutes. The pellet was then resuspended in 50 mM Tris pH9.0 (approximately 12.5 ml of buffer per gram of pellet). The suspension was mixed well until it was fully homogeneous. Lysis of cells was carried out by sonication (6×30 seconds on, 6×30 seconds off). DDM was added to the cell lysate to 1% final concentration and the mixture was incubated at 37° C. for 45 minutes with frequent mixing. The lysate was pelleted by centrifugation at 20,000 g for 45 minutes and the supernatant was then separated. The supernatant which contained both forms of monomers and a mixture of different oligomers was purified by column chromatography as described below.

The sample was diluted 1:1 with 50 mM Tris, 500 mM NaCl, 0.1% DDM, pH 8.0 (buffer A) and applied to a 5 ml His Crude FF column (GE Healthcare). The column was washed until a stable baseline of 10 column volumes was maintained. Loosely bound protein was removed by washing the column with 15 mM Imidazole (Sigma/Aldrich Biopuriss grade) until a stable base line was maintained. Elution was carried out with 200 mM imidazole (Sigma/Aldrich Biopuriss grade).

The elution peak was pooled and heated to 85° C. for 15 minutes to remove heat unstable contaminated proteins. The heated solution was then subjected to centrifugation at 20,000 g for 10 minutes and the pellet discarded. The supernatant was subjected to gel filtration on a 120 ml Sephadex S200 column (GE Healthcare) in buffer A at 1 ml/min. The sample was eluted at approximately 60 ml volume. The elution peak was run on a 10% TGX (Bio Rad) to confirm the presence of the heteropore of interest MspA 1. Identified fractions were pooled and carried forward for further purification.

The sample was loaded onto a 1 ml His HP column (GE Healthcare) in buffer A. Once a stable baseline was maintained for 10 column volumes, the column was washed with 15 mM imidazole (Sigma/Aldrich Biopuriss grade). Elution of MspA 1 was carried out with a gradient of 15 mM imidazole to 80 mM imidazole (Sigma/Aldrich Biopuriss grade) at 1 ml/min over 60 minutes. Other conformations of hetero pores containing more than one His tag were eluted by increasing the imidazole concentration in the elution to 100 mM.

The hetero-oligomeric nanopores listed below were also made using this method. In each case, DNA encoding the non-tagged subunits was cloned in the pRham vector and the DNA encoding the subunit containing BasTL-H6 was cloned in the pT7 vector and purified as explained in the above example. MspA 2=MspA—((Del-L74/G75/D118/L119) D56N/E59R/L88N/D90N/D91N/N108P/Q126R/D134R/E139K) 7 ((Del-L74/G75/D118/L119) D56N/E59R/L88N/D91N/N108P/Q126R/D134R/E139K/BasTL/H6) 1 (SEQ ID NO: 2 with the following mutations in seven monomers D56N/E59R/L88N/D90N/D91N/N108P/Q126R/D134R/E139K and deletion, of the following amino acids in seven monomers L74/G75/D118/L119 and the following mutations in one monomer of D56N/E59R/L88N/D91N/N108P/Q126R/D134R/E139K/BasTL/H6, where the BasTL has SEQ ID NO: 26, the six histidines (H6) are attached at the 3′ end of the BasTL at the C-terminus and is attached at the C-terminus, deletions of the following amino acids in one monomer of L74/G75/D118/L119) MspA 3=MspA—((Del-L74/G75/D118/L119) D56N/E59R/L88N/D90N/D91N/N108P/Q126R/D134R/E139K) 7 ((Del-L74/G75/D118/L119) D56N/E59R/L88N/D91G/N108P/Q126R/D134R/E139K/BasTL/H6) 1 (SEQ ID NO: 2 with the following mutations in seven monomers D56N/E59R/L88N/D90N/D91N/N108P/Q126R/D134R/E139K and deletion, of the following amino acids in seven monomers L74/G75/D118/L119 and the following mutations in one monomer of D56N/E59R/L88N/D91G/N108P/Q126R/D134R/E139K/BasTL/H6, where the BasTL has SEQ ID NO: 26, the six histidines (H6) are attached at the 3′ end of the BasTL at the C-terminus and is attached at the C-terminus, deletions of the following amino acids in one monomer of L74/G75/D118/L119) MspA 4=MspA—((Del-L74/G75/D118/L119) D56F/E59R/L88N/D90N/D91N/N108P/Q126R/D134R/E139K) 7 ((Del-L74/G75/D118/L119) D56F/E59R/L88N/D91N/N108P/Q126R/D134R/E139K/BasTL/H6) 1 (SEQ ID NO: 2 with the following mutations in seven monomers D56F/E59R/L88N/D90N/D91N/N108P/Q126R/D134R/E139K and deletion, of the following amino acids in seven monomers L74/G75/D118/L119 and the following mutations in one monomer of D56F/E59R/L88N/D91N/N108P/Q126R/D134R/E139K/BasTL/H6, where the BasTL has SEQ ID NO: 26, the six histidines (H6) are attached at the 3′ end of the BasTL at the C-terminus and is attached at the C-terminus, deletions of the following amino acids in one monomer of L74/G75/D118/L119) MspA 5=MspA—((Del-L74/G75/D118/L119) D56F/E59R/L88N/D90N/D91N/N108P/Q126R/D134R/E139K) 7 ((Del-L74/G75/D118/L119) D56F/E59R/L88N/D91G/N108P/Q126R/D134R/E139K/BasTL/H6) 1 (SEQ ID NO: 2 with the following mutations in seven monomers D56F/E59R/L88N/D90N/D91N/N108P/Q126R/D134R/E139K and deletion, of the following amino acids in seven monomers L74/G75/D118/L119 and the following mutations in one monomer of D56F/E59R/L88N/D91G/N108P/Q126R/D134R/E139K/BasTL/H6, where the BasTL has SEQ ID NO: 26, the six histidines (H6) are attached at the 3′ end of the BasTL at the C-terminus and is attached at the C-terminus, deletions of the following amino acids in one monomer of L74/G75/D118/L119) MspA 7=MspA—((Del-L74/G75/D118/L119) (D56N/E59R/L88N/D90N/D91N/N108P/Q126R/D134R/E139K) 7 ((Del-L74/G75/D118/L119) (D56N/E59R/L88N/D91Q/N108P/Q126R/D134R/E139K/BasTL/H6) 1 (SEQ ID NO: 2 with the following mutations in seven monomers D56N/E59R/L88N/D90N/D91N/N108P/Q126R/D134R/E139K and deletion, of the following amino acids in seven monomers L74/G75/D118/L119 and the following mutations in one monomer of D56N/E59R/L88N/D91Q/N108P/Q126R/D134R/E139K/BasTL/H6, where the BasTL has SEQ ID NO: 26, the six histidines (H6) are attached at the 3′ end of the BasTL at the C-terminus and is attached at the C-terminus, deletions of the following amino acids in one monomer of L74/G75/D118/L119)

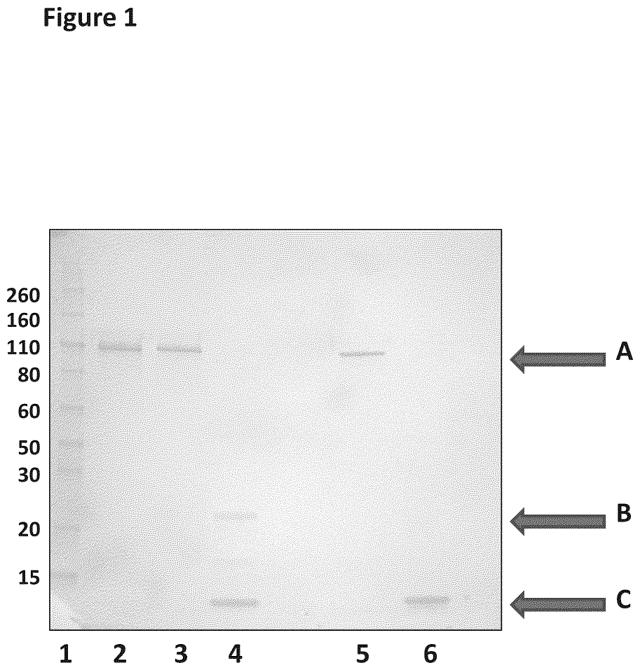

An example of the purified MspA 2 is shown in FIG. 1. Proteins were analysed in a 10% TGX gel (BioRad) and visualised with coomasie staining. Lane 3 shows the purified MspA—2 with a band corresponding to a hetero-oligomeric pore made up of 8 monomer units which have oligomerised. Lane 4 shows the purified MspA—2 after heat treatment which broke the pore down into its monomer units. Two bands were observed in Lane 4-one which corresponded to the monomer unit with the BasTL-H6 attached (band B) and one which corresponded to the monomer unit without the BasTL-H6 attached. This illustrated that the purified pore in lane 8 broke down into two different monomer units which was evidence that the MspA—2 heteropore had been formed.

### Example 2

This example describes a control experiment which showed that co-transformation and subsequent controlled expression of the two monomers was necessary to obtain the hetero pores of interest. Two subunits which were transformed individually, but grown together in the same culture did not produce hetero-oligomeric pores.

DNA encoding polypeptide (SEQ ID NO: 27) MspA—((Del-L74/G75/D118/L119) D56N/E59R/L88N/D90N/D91N/Q126R//D134R/E139K) (SEQ ID NO: 2 with the following mutations in the monomer D56N/E59R/L88N/D90N/D91N/Q126R//D134R/E139K and deletion, of the following amino acids in the monomers L74/G75/D118/L119) and DNA encoding polypeptide (SEQ ID NO: 28) MspA—((Del-L74/G75/D118/L119) D56N/E59R/L88N/D91N/Q126R//D134R/E139K) (SEQ ID NO: 2 with the following mutations in the monomer D56N/E59R/L88N/D91N/Q126R//D134R/E139K and deletion, of the following amino acids in the monomers L74/G75/D118/L119) were cloned into the pT7 vector containing ampicillin resistance gene. The two DNA encoding polypeptides were transformed separately into BL21 DE3 PlysS competent cells. Starter cultures were made by inoculating 10 ml of TB media containing 0.1 mg/ml ampicillin with a single colony from each transformant. Starter cultures were grown at 37° C. with agitation to an OD600 of 0.8. 500 ml of TB media containing 0.1 mg/ml ampicillin was inoculated with both subunits by adding 1 ml of starter cultures of each subunit. The inoculated culture was grown to an OD600 of 0.8. Once the desired OD600 was reached, the culture was allowed to cool to 18° C. and induced by the addition of 1 mM IPTG. The culture was then incubated at 18° C. with agitation for 8 hours.

Following incubation, the sample was lysed as standard-culture was pelleted by centrifugation at 6,000 g for 30 minutes and the pellet was resuspended in 50 mM Tris pH 9 (approximately 12.5 ml per gram of pellet). The suspension was mixed until fully homogeneous. Lysis was carried out by sonication (6×30 seconds on, 6×30 seconds off). DDM was added to the lysate (1% final concentration) before incubation at 37° C. for 45 minutes with frequent mixing. The lysate was pelleted by centrifugation at 20,000 g for 45 minutes. The samples were then visualised on 7.5% Tris HCl gel pre and post heating to 85° C. to determine whether hetero pore formation occurred.

For all of the samples tested no hetero pores were observed. This confirmed that co-transformation of the same bacterial cell with the DNA encoding both subunits and subsequent induction of the expression in a controlled manner was necessary to obtain the heteropore of interest.

### Example 3

This example described a number of different conditions which were investigated in order to identify the optimised ratio of IPTG:Rhamnose that favoured stable 7:1 hetero pore formation.

In brief, pRham vector containing DNA encoding the polypeptide (SEQ ID NO: 29) for MspA—((Del-L74/G75/D118/L119) D56N/E59R/L88N/D90N/D91N/N108P/Q126R/D134R/E139K) (SEQ ID NO: 2 with the following mutations in the monomer D56N/E59R/L88N/D90N/D91N/Q126R/D134R/E139K and deletion, of the following amino acids in the monomers L74/G75/D118/L119) and the pT7 vector containing DNA encoding the polypeptide (SEQ ID NO: 30) for MspA—((Del-L74/G75/D118/L119) D56N/E59R/L88N/D91N/N108P/Q126R/D134R/E139K/BastL/H6) (SEQ ID NO: 2 with the following mutations in the monomer D56N/E59R/L88N/D90N/D91N/Q126R/D134R/E139K where the BasTL has SEQ ID NO: 26 and is attached at the C-terminus, the six histidines (H6) are attached at the 3′ end of the BasTL at the C-terminus and deletions of the following amino acids in the monomers L74/G75/D118/L119) were co-transformed to Lemo21 (DE3) competent E. coli cells and grown in 500 ml of TB media containing 0.1 mg/ml ampicillin and 0.034 mg/ml kanamycin as detailed above in Example 1. Once the culture had reached an OD600 of 0.85, it was aliquoted into 14×10 ml subcultures. Each subculture was induced with differing amounts of Rhamnose and IPTG as shown below:
1) 0.5% Rhamnose/1 mM IPTG
2) 1% Rhamnose/1 mM IPTG
3) 1% Rhamnose/0.5 mM IPTG
4) 1.5% Rhamnose/1 mM IPTG
5) 1.5% Rhamnose/0.5 mM IPTG
6) 2% Rhamnose/1 mM IPTG
7) 2% Rhamnose/0.5 mM IPTG
8) 3% Rhamnose/1 mM IPTG
9) 3% Rhamnose/0.5 mM IPTG
10) 1% Rhamnose/0.25 mM IPTG
11) 0.5% Rhamnose/0.5 mM IPTG
12) 0.5% Rhamnose/0.25 mM IPTG
13) 0% Rhamnose/1 mM IPTG
14) 0.5% Rhamnose/0 mM IPTG

Induction was carried out for approximately 18 hours with agitation at 18° C. The cultures were pelleted by centrifugation at 6,000 g for 30 minutes. Pellets were resuspended in 50 mM Tris pH 9 (approx 12.5 ml per gram of pellet). Suspensions were mixed until fully homogeneous. Lysis was carried out by sonication (6×30 seconds on, 6×30 seconds off). DDM was added to the lysates (1% final concentration) before incubation at 37° C. for 45 minutes with frequent mixing. The lysates were pelleted by centrifugation at 20,000 g for 45 minutes.

Following solubilisation the samples were batch purified on Qiagen NiNTA sepharose beads as instructed by the manufacturer. The elution was split into two aliquots. One aliquot was kept unheated and the other aliquot was heated to 85° C. to assess heat stability before both samples were visualised on 10% TGX (bio rad) gel. Gel analysis confirmed that condition 5 with 1.5% Rhamnose and 0.5 mM IPTG gave the most amount of stable heteropore of interest MspA—2.

### Example 4

This example describes how a number of different tags were investigated for their ability to alter the oligomerisation efficiency of monomers. For the purpose of this experiment, different tags of various lengths and charges were added to the N or C termini of the MspA monomer MspA—(G75S/G77S/L88N/D90N/D91N/D93N/D118R/Q126R/D134R/E139K) (SEQ ID NO: 2 with the following mutations G75S/G77S/L88N/D90N/D91N/D93N/D118R/Q126R/D134R/E139K). DNA for such constructs was generated by PCR methods known in the art. The circular DNA plasmid containing the T7 promoter and DNA of MspA—(G75S/G77S/L88N/D90N/D91N/D93N/D118R/Q126R/D134R/E139K) (SEQ ID NO: 2 with the following mutations G75S/G77S/L88N/D90N/D91N/D93N/D118R/Q126R/D134R/E139K) with or without tags was prepared by QIAGEN Plasmid Midi Kit (catalogue number 12145) and the concentration of samples adjusted to 400 ng/uL. Proteins were generated by coupled in vitro transcription and translation (IVTT) by using an Escherichia coli T7-S30 extract system for circular DNA (Promega, no. L1130). Prepared rabbit red blood cell membranes (rRBCM) were used to facilitate oligomerisation of monomers into their oligomer. DNA (16 μl, 400 ng/μl) of the monomer of interest was used in a 100 μL IVTT reaction which generated homo-oligomeric pores where all monomers in the oligomer are identical. DNA for two monomers (eg: a tagged and untagged versions) were mixed together in specific ratios and the DNA mixture (16 μl, 400 ng/uL) was used in a 100 μL IVTT reaction to generate hetero-oligomeric pores. Depending on the ratio of DNA (untagged:tagged) used, a range of hetero-oligomers was produced (eg: 8:0, 7:1, 6:2, 5:3 etc.) within the reaction. Therefore, the ratio of DNA between the two monomers was adjusted to bias the desired oligomeric pores produced (eg: more of 8:0, 7:1 rather than 3:5, 2:6).

To generate proteins in IVTT, 50 μL of rRBCM (10 mM Mops pH 7.4, 2 mg/mL) were centrifuged in 1.5 mL Eppendorf tube and the supernatant was removed. The pellet was resuspended in IVTT reagents required to carry out a 100 μL IVTT reaction. The complete amino acid mixture (1 mM) minus cysteine and the complete amino acid mixture (1 mM) minus methionine, supplied in the kit, were mixed in equal volumes which produced the working amino acid solution required to generate high concentrations of the proteins. 10 μL of this amino acid mixture was mixed with the premix solution (40 μL), L-[35S] methionine (2 μL, PerkinElmer, product code NEG009A001MC), plasmid DNA as mentioned above (16 μL, 400 ng/uL), and T7-S30 extract (30 μL) supplemented with rifampicin (20 μg/ml final). Protein synthesis was carried out for 1.5 hours at 37° C. to produce radiolabelled IVTT protein. Due to the presence of rRBCM and E. coli cell membranes in the IVTT reaction mixture, monomers synthesized within the reaction assembled on the membranes to produce homo or hetero-oligomeric nanopores. After centrifugation, the resulting membrane pellets were washed with MBSA (100 μL, 10 mM MOPS titrated with NaOH, 150 mM NaCl, pH 7.4, containing 1 mg/ml BSA) and the pellets were resuspended in 100 μL of 1× laemmli sample buffer. The sample was divided into two and one sample was heated at 85° C. for 15 minutes to assess the heat stability of the oligomers. Heated and/or unheated samples were subjected to electrophoresis in 5% or 7.5% Tris HCl gels with TGS running buffer for 16 hours at 50 mV. The gels were dried and subjected to autoradiography to visualize the proteins.

### 4.1—Effect of the Length of the Tag

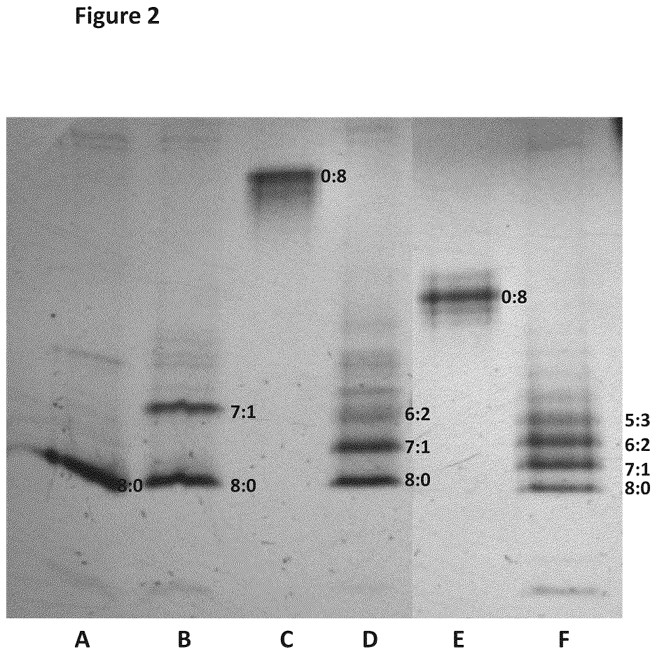

By changing the length of the tag, it was possible to bias the production of the desired oligomer. In the following example, MspA—(G75S/G77S/L88N/D90N/D91N/D93N/D118R/Q126R/D134R/E139K) (SEQ ID NO: 2 with the following mutations G75S/G77S/L88N/D90N/D91N/D93N/D118R/Q126R/D134R/E139K) was tagged with full length BasTL (SEQ ID NO: 26) and truncated versions of the BasTL. Tagged monomers were either oligomerised into their homo-oligomers or mixed with untagged MspA—(G75S/G77S/L88N/D90N/D91N/D93N/D118R/Q126R/D134R/E139K) (SEQ ID NO: 2 with the following mutations G75S/G77S/L88N/D90N/D91N/D93N/D118R/Q126R/D134R/E139K) to produce hetero-oligomers. Heated samples were run on a 7.5% Tris HCl gel (see FIG. 2).

Lane A of FIG. 2 shows the MspA—(G75S/G77S/L88N/D90N/D91N/D93N/D118R/Q126R/D134R/E139K) 8 homo-oligomer. Lanes C and E show homo-oligomers for MspA—(G75S/G77S/L88N/D90N/D91N/D93N/D118R/Q126R/D134R/E139K/BasTL (minus20 aa)) 8 and MspA—(G75S/G77S/L88N/D90N/D91N/D93N/D118R/Q126R/D134R/E139K/BasTL (minus40 aa)) 8 respectively. Although the same amount of DNA was used in each experiment, the amount of homo-oligomer produced was diminished when a long polypeptide tag (BasTL in this instance) was attached (lanes C and E of FIG. 2) compared to the untagged version (lane A which showed a much darker band in FIG. 2). Lanes B, D and F of FIG. 2 showed hetero-oligomers produced when MspA—(G75S/G77S/L88N/D90N/D91N/D93N/D118R/Q126R/D134R/E139K) was mixed with MspA—(G75S/G77S/L88N/D90N/D91N/D93N/D118R/Q126R/D134R/E139K/BasTL), MspA—(G75S/G77S/L88N/D90N/D91N/D93N/D118R/Q126R/D134R/E139K/BasTL (minus20 aa)) and MspA—(G75S/G77S/L88N/D90N/D91N/D93N/D118R/Q126R/D134R/E139K/BasTL (minus40 aa)) respectively. Each band in each lane of FIG. 2 has the ratio of the monomer units which produced the band in the gel (untagged monomer: tagged monomer) marked next to the corresponding band. The results showed that by changing the length of the tag, it was possible to manipulate the production of desired hetero-oligomers.

### 4.2 Effect of Charges in the Tag

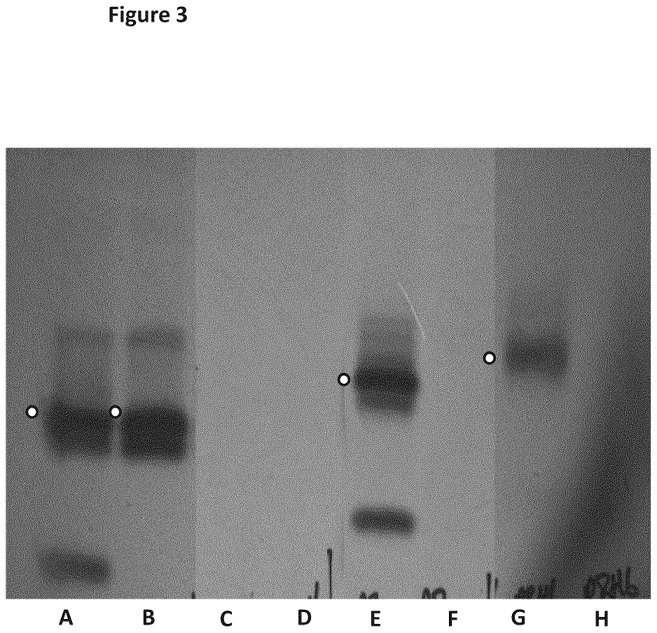

MspA—(G75S/G77S/L88N/D90N/D91N/D93N/D118R/Q126R/D134R/E139K) monomer was modified with various charged tags of different lengths at the N or C termini. Ability of these monomers to form pores was analysed in IVTT experiments. The full list of different tags tested included R8SG, D6SG, R6SG, R8, NGDSD6SG, D4SG, R4SG, D4, D6, GDSGD4SG, R4H6, D4H6, D8, R6, D10, R4, D4, D8H6, R8H9, D10H6, and R6H6. The MspA—(G75S/G77S/L88N/D90N/D91N/D93N/D118R/Q126R/D134R/E139K) monomer labelled with D10H6, R8 or R8H6 tags described in more detail below and in FIG. 3.

FIG. 3 shows 5% Tris HCl gel which compares oligomerisation of MspA—(G75S/G77S/L88N/D90N/D91N/D93N/D118R/Q126R/D134R/E139K) monomers when a variety of different tags were attached to the C terminus. White circles in FIG. 3 indicate the MspA homo-oligomer formed in each lane. Lanes A shows that the MspA—(G75S/G77S/L88N/D90N/D91N/D93N/D118R/Q126R/D134R/E139K) monomer without any tag at the C terminus oligomerised well. Lane B shows that the oligomer formed from the MspA—(G75S/G77S/L88N/D90N/D91N/D93N/D118R/Q126R/D134R/E139K) monomer was stable at least up to 85° C. Lane C shows that D10H6 tag at the C terminus of the MspA—(G75S/G77S/L88N/D90N/D91N/D93N/D118R/Q126R/D134R/E139K) monomer prevented it from oligomerising completely or at least the oligomer formed was not SDS stable. Lanes E and F show that although R8 tag at the C terminus of the MspA—(G75S/G77S/L88N/D90N/D91N/D93N/D118R/Q126R/D134R/E139K) monomer oligomerised well, heat stability of the oligomer had been compromised by that addition of the tag. Lane G shows that R8H6 tag at the C terminus of the MspA—(G75S/G77S/L88N/D90N/D91N/D93N/D118R/Q126R/D134R/E139K) monomer diminished its oligomerisation ability compared to the untagged monomer in lane A. Lane H shows that similar to the R8 tag, the R8H6 tag also compromised the heat stability of its oligomer.

1-44. (canceled)

45. A hetero-oligomeric transmembrane pore comprising a first monomer and a second monomer at a specific stoichiometric ratio in the range of 1:13 to 13:1, wherein the specific stoichiometric ratio is not 1:1, wherein the first monomer is different from the second monomer, and wherein:
(i) the first monomer comprises SEQ ID NO: 2 while the second monomer comprises a variant of SEQ ID NO: 2, or the first monomer comprises a variant of SEQ ID NO: 2 while the second monomer comprises SEQ ID NO: 2, or the first monomer comprises a first variant of SEQ ID NO: 2 while the second monomer comprises a second variant of SEQ ID NO: 2 that is different from the first variant; or
(ii) the first monomer comprises SEQ ID NO: 32 while the second monomer comprises a variant of SEQ ID NO: 32, or the first monomer comprises a variant of SEQ ID NO: 32 while the second monomer comprises SEQ ID NO: 32, or the first monomer comprises a first variant of SEQ ID NO: 32 while the second monomer comprises a second variant of SEQ ID NO: 32 that is different from the first variant.

46. The hetero-oligomeric transmembrane pore of claim 45, wherein the hetero-oligomeric transmembrane pore is in a membrane.

47. The hetero-oligomeric transmembrane pore of claim 45, wherein the membrane is an amphiphilic layer, a copolymer or a solid state layer.

48. The hetero-oligomeric transmembrane pore of claim 45, wherein the specific stoichiometric ratio of the first monomer to the second monomer is at least 2:1, 3:1, 4:1, 5:1, 6:1, 7:1, or 8:1.

49. The hetero-oligomeric transmembrane pore of claim 45, wherein the specific stoichiometric ratio of the first monomer to the second monomer is 1:2, 2:1, 1:3, 3:1, 1:4, 2:3, 3:2, 4:1, 1:5, 1:6, 2:5, 3:4, 4:3, 5:2, 1:7, 3:5, 5:3, 1:8, 2:7, 4:5, 5:4, 7:2, 1:9, 3:7, 7:3, 5:1, 6:1, 7:1, 8:1, 9:1, 1:10, 2:9, 3:8, 4:7, 5:6, 6:5, 7:4, 8:3, 9:2, 10:1, 1:11, 5:7, 7:5, 11:1, 1:12, 2:11, 3:10, 4:9, 5:8, 6:7, 7:6, 8:5, 9:4, 10:3, 11:2, 12:1, 1:13, 3:11, 5:9, 9:5, 11:3, or 13:1.

50. The hetero-oligomeric transmembrane pore of claim 45, wherein the variant of SEQ ID NO: 2 comprises an amino acid mutation at one or more of the positions corresponding to positions 88, 90, 91, 92, 93, 102, 103 and 105 of SEQ ID NO: 2.

51. The hetero-oligomeric transmembrane pore of claim 45, wherein the variant of SEQ ID NO: 2 comprises a negatively charged amino acid at one or more of the positions corresponding to positions 90, 91, 93 and 105 of SEQ ID NO: 2

52. The hetero-oligomeric transmembrane pore of claim 45, wherein the variant of SEQ ID NO: 32 comprises an amino acid mutation at one or more of the positions corresponding to positions N40, D43, E44, S54, S57, Q62, R97, E101, E124, E131, R142, T150 and R192 of SEQ ID NO: 32.

53. A kit for characterising a target analyte comprising:
(a) a hetero-oligomeric transmembrane pore of claim 45; and
(b) the components to form a membrane.

54. The kit of claim 53, wherein the membrane is an amphiphilic layer, a triblock copolymer membrane, or a solid state layer.

55. An apparatus for characterising target analytes in a sample, comprising:
(a) a plurality of the hetero-oligomeric transmembrane pores of claim 45; and
(b) a plurality of membranes.

56. The apparatus of claim 55, wherein the membrane is an amphiphilic layer, a triblock copolymer membrane, or a solid state layer.


SEQUENCE LISTING (32 sequences)

SEQ ID NO: 1 - DNA, 558 bp, Mycobacterium smegmatis
  source 1..558: mol_type = genomic DNA
atgggcctggataacgaacttagcctggtggacggccaagatcgcacgctgacggtgcaacaatgggataccttcctgaatggtgtgtttccgctggatcgtaaccgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcgatgacggtgatattaccgcaccgccgtttggcctgaacagcgtgattacgccgaacctgtttccgggtgttagcatctctgccgatctgggcaacggtccgggcattcaagaagtggcaacctttagtgtggacgtttccggcgctgaaggcggtgtcgcggtgtctaatgcccacggtaccgttacgggcgcggccggcggtgtcctgctgcgtccgttcgcgcgcctgattgcgagcaccggcgactctgttacgacctatggcgaaccgtggaatatgaactaa

SEQ ID NO: 2 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGAEGGVAVSNAHGTVTGAAGGVLLRPFARLIASTGDSVTTYGEPWNMN

SEQ ID NO: 3 - DNA, 885 bp, synthetic construct
  misc_feature 1..885: note = Synthetic polynucleotide
  source 1..885: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaaaactatatgagtactttaacttatggattcaacggtaatgttactggtgatgatacaggaaaaattggcggccttattggtgcaaatgtttcgattggtcatacactgaactatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 4 - protein, 293 aa, synthetic construct
  REGION 1..293: note = Synthetic polypeptide
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYMSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 6 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 183 aa, Mycobacterium smegmatis
VDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 8 - DNA, 1830 bp, Bacillus subtilis
  source 1..1830: mol_type = genomic DNA; note = phage phi29
atgaaacacatgccgcgtaaaatgtatagctgcgcgtttgaaaccacgaccaaagtggaagattgtcgcgtttgggcctatggctacatgaacatcgaagatcattctgaatacaaaatcggtaacagtctggatgaatttatggcatgggtgctgaaagttcaggcggatctgtacttccacaacctgaaatttgatggcgcattcattatcaactggctggaacgtaatggctttaaatggagcgcggatggtctgccgaacacgtataataccattatctctcgtatgggccagtggtatatgattgatatctgcctgggctacaaaggtaaacgcaaaattcataccgtgatctatgatagcctgaaaaaactgccgtttccggtgaagaaaattgcgaaagatttcaaactgacggttctgaaaggcgatattgattatcacaaagaacgtccggttggttacaaaatcaccccggaagaatacgcatacatcaaaaacgatatccagatcatcgcagaagcgctgctgattcagtttaaacagggcctggatcgcatgaccgcgggcagtgatagcctgaaaggtttcaaagatatcatcacgaccaaaaaattcaaaaaagtgttcccgacgctgagcctgggtctggataaagaagttcgttatgcctaccgcggcggttttacctggctgaacgatcgtttcaaagaaaaagaaattggcgagggtatggtgtttgatgttaatagtctgtatccggcacagatgtacagccgcctgctgccgtatggcgaaccgatcgtgttcgagggtaaatatgtttgggatgaagattacccgctgcatattcagcacatccgttgtgaatttgaactgaaagaaggctatattccgaccattcagatcaaacgtagtcgcttctataagggtaacgaatacctgaaaagctctggcggtgaaatcgcggatctgtggctgagtaacgtggatctggaactgatgaaagaacactacgatctgtacaacgttgaatacatcagcggcctgaaatttaaagccacgaccggtctgttcaaagatttcatcgataaatggacctacatcaaaacgacctctgaaggcgcgattaaacagctggccaaactgatgctgaacagcctgtatggcaaattcgcctctaatccggatgtgaccggtaaagttccgtacctgaaagaaaatggcgcactgggttttcgcctgggcgaagaagaaacgaaagatccggtgtataccccgatgggtgttttcattacggcctgggcacgttacacgaccatcaccgcggcccaggcatgctatgatcgcattatctactgtgataccgattctattcatctgacgggcaccgaaatcccggatgtgattaaagatatcgttgatccgaaaaaactgggttattgggcccacgaaagtacgtttaaacgtgcaaaatacctgcgccagaaaacctacatccaggatatctacatgaaagaagtggatggcaaactggttgaaggttctccggatgattacaccgatatcaaattcagtgtgaaatgcgccggcatgacggataaaatcaaaaaagaagtgaccttcgaaaacttcaaagttggtttcagccgcaaaatgaaaccgaaaccggtgcaggttccgggcggtgtggttctggtggatgatacgtttaccattaaatctggcggtagtgcgtggagccatccgcagttcgaaaaaggcggtggctctggtggcggttctggcggtagtgcctggagccacccgcagtttgaaaaataataa

SEQ ID NO: 9 - protein, 608 aa, Bacillus subtilis
  source 1..608: note = phage phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 10 - DNA, 1380 bp, Escherichia coli
  source 1..1380: mol_type = genomic DNA
atgatgaacgatggcaaacagcagagcaccttcctgtttcatgattatgaaaccttcggtacccatccggccctggatcgtccggcgcagtttgcggccattcgcaccgatagcgaattcaatgtgattggcgaaccggaagtgttttattgcaaaccggccgatgattatctgccgcagccgggtgcggtgctgattaccggtattaccccgcaggaagcgcgcgcgaaaggtgaaaacgaagcggcgtttgccgcgcgcattcatagcctgtttaccgtgccgaaaacctgcattctgggctataacaatgtgcgcttcgatgatgaagttacccgtaatatcttttatcgtaacttttatgatccgtatgcgtggagctggcagcatgataacagccgttgggatctgctggatgtgatgcgcgcgtgctatgcgctgcgcccggaaggcattaattggccggaaaacgatgatggcctgccgagctttcgtctggaacatctgaccaaagccaacggcattgaacatagcaatgcccatgatgcgatggccgatgtttatgcgaccattgcgatggcgaaactggttaaaacccgtcagccgcgcctgtttgattatctgtttacccaccgtaacaaacacaaactgatggcgctgattgatgttccgcagatgaaaccgctggtgcatgtgagcggcatgtttggcgcctggcgcggcaacaccagctgggtggccccgctggcctggcacccggaaaatcgtaacgccgtgattatggttgatctggccggtgatattagcccgctgctggaactggatagcgataccctgcgtgaacgcctgtataccgccaaaaccgatctgggcgataatgccgccgtgccggtgaaactggttcacattaacaaatgcccggtgctggcccaggcgaacaccctgcgcccggaagatgcggatcgtctgggtattaatcgccagcattgtctggataatctgaaaatcctgcgtgaaaacccgcaggtgcgtgaaaaagtggtggcgatcttcgcggaagcggaaccgttcaccccgagcgataacgtggatgcgcagctgtataacggcttctttagcgatgccgatcgcgcggcgatgaaaatcgttctggaaaccgaaccgcgcaatctgccggcgctggatattacctttgttgataaacgtattgaaaaactgctgtttaattatcgtgcgcgcaattttccgggtaccctggattatgccgaacagcagcgttggctggaacatcgtcgtcaggttttcaccccggaatttctgcagggttatgcggatgaactgcagatgctggttcagcagtatgccgatgataaagaaaaa

SEQ ID NO: 11 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 12 - DNA, 804 bp, Escherichia coli
  source 1..804: mol_type = genomic DNA
atgaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggatatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 13 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 14 - DNA, 1275 bp, Thermus thermophilus
  source 1..1275: mol_type = genomic DNA
atgtttcgtcgtaaagaagatctggatccgccgctggcactgctgccgctgaaaggcctgcgcgaagccgccgcactgctggaagaagcgctgcgtcaaggtaaacgcattcgtgttcacggcgactatgatgcggatggcctgaccggcaccgcgatcctggttcgtggtctggccgccctgggtgcggatgttcatccgtttatcccgcaccgcctggaagaaggctatggtgtcctgatggaacgcgtcccggaacatctggaagcctcggacctgtttctgaccgttgactgcggcattaccaaccatgcggaactgcgcgaactgctggaaaatggcgtggaagtcattgttaccgatcatcatacgccgggcaaaacgccgccgccgggtctggtcgtgcatccggcgctgacgccggatctgaaagaaaaaccgaccggcgcaggcgtggcgtttctgctgctgtgggcactgcatgaacgcctgggcctgccgccgccgctggaatacgcggacctggcagccgttggcaccattgccgacgttgccccgctgtggggttggaatcgtgcactggtgaaagaaggtctggcacgcatcccggcttcatcttgggtgggcctgcgtctgctggctgaagccgtgggctataccggcaaagcggtcgaagtcgctttccgcatcgcgccgcgcatcaatgcggcttcccgcctgggcgaagcggaaaaagccctgcgcctgctgctgacggatgatgcggcagaagctcaggcgctggtcggcgaactgcaccgtctgaacgcccgtcgtcagaccctggaagaagcgatgctgcgcaaactgctgccgcaggccgacccggaagcgaaagccatcgttctgctggacccggaaggccatccgggtgttatgggtattgtggcctctcgcatcctggaagcgaccctgcgcccggtctttctggtggcccagggcaaaggcaccgtgcgttcgctggctccgatttccgccgtcgaagcactgcgcagcgcggaagatctgctgctgcgttatggtggtcataaagaagcggcgggtttcgcaatggatgaagcgctgtttccggcgttcaaagcacgcgttgaagcgtatgccgcacgtttcccggatccggttcgtgaagtggcactgctggatctgctgccggaaccgggcctgctgccgcaggtgttccgtgaactggcactgctggaaccgtatggtgaaggtaacccggaaccgctgttcctg

SEQ ID NO: 15 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 16 - DNA, 738 bp, unidentified
  source 1..738: mol_type = genomic DNA; note = Bacteriophage lambda
tccggaagcggctctggtagtggttctggcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatctggctctggttccggcagcggttccgga

SEQ ID NO: 17 - protein, 226 aa, unidentified
  source 1..226: note = Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 18 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 19 - protein, 707 aa, unidentified
  source 1..707: note = Cenarchaeum symbiosum
MRISELDIPRPAIEFLEGEGYKKLYPPQAAAAKAGLTDGKSVLVSAPTASGKTLIAAIAMISHLSRNRGKAVYLSPLRALAAEKFAEFGKIGGIPLGRPVRVGVSTGDFEKAGRSLGNNDILVLTNERMDSLIRRRPDWMDEVGLVIADEIHLIGDRSRGPTLEMVLTKLRGLRSSPQVVALSATISNADEIAGWLDCTLVHSTWRPVPLSEGVYQDGEVAMGDGSRHEVAATGGGPAVDLAAESVAEGGQSLIFADTRARSASLAAKASAVIPEAKGADAAKLAAAAKKIISSGGETKLAKTLAELVEKGAAFHHAGLNQDCRSVVEEEFRSGRIRLLASTPTLAAGVNLPARRVVISSVMRYNSSSGMSEPISILEYKQLCGRAGRPQYDKSGEAIVVGGVNADEIFDRYIGGEPEPIRSAMVDDRALRIHVLSLVTTSPGIKEDDVTEFFLGTLGGQQSGESTVKFSVAVALRFLQEEGMLGRRGGRLAATKMGRLVSRLYMDPMTAVTLRDAVGEASPGRMHTLGFLHLVSECSEFMPRFALRQKDHEVAEMMLEAGRGELLRPVYSYECGRGLLALHRWIGESPEAKLAEDLKFESGDVHRMVESSGWLLRCIWEISKHQERPDLLGELDVLRSRVAYGIKAELVPLVSIKGIGRVRSRRLFRGGIKGPGDLAAVPVERLSRVEGIGATLANNIKSQLRKGG

SEQ ID NO: 20 - protein, 720 aa, Thermococcus gammatolerans
MKVDELPVDERLKAVLKERGIEELYPPQAEALKSGALEGRNLVLAIPTASGKTLVSEIVMVNKLIQEGGKAVYLVPLKALAEEKYREFKEWEKLGLKVAATTGDYDSTDDWLGRYDIIVATAEKFDSLLRHGARWINDVKLVVADEVHLIGSYDRGATLEMILTHMLGRAQILALSATVGNAEELAEWLDASLVVSDWRPVQLRRGVFHLGTLIWEDGKVESYPENWYSLVVDAVKRGKGALVFVNTRRSAEKEALALSKLVSSHLTKPEKRALESLASQLEDNPTSEKLKRALRGGVAFHHAGLSRVERTLIEDAFREGLIKVITATPTLSAGVNLPSFRVIIRDTKRYAGFGWTDIPVLEIQQMMGRAGRPRYDKYGEAIIVARTDEPGKLMERYIRGKPEKLFSMLANEQAFRSQVLALITNFGIRSFPELVRFLERTFYAHQRKDLSSLEYKAKEVVYFLIENEFIDLDLEDRFIPLPFGKRTSQLYIDPLTAKKFKDAFPAIERNPNPFGIFQLIASTPDMATLTARRREMEDYLDLAYELEDKLYASIPYYEDSRFQGFLGQVKTAKVLLDWINEVPEARIYETYSIDPGDLYRLLELADWLMYSLIELYKLFEPKEEILNYLRDLHLRLRHGVREELLELVRLPNIGRKRARALYNAGFRSVEAIANAKPAELLAVEGIGAKILDGIYRHLGIEKRVTEEKPKRKGTLEDFLR

SEQ ID NO: 21 - protein, 799 aa, Methanospirillum hungatei
MEIASLPLPDSFIRACHAKGIRSLYPPQAECIEKGLLEGKNLLISIPTASGKTLLAEMAMWSRIAAGGKCLYIVPLRALASEKYDEFSKKGVIRVGIATGDLDRTDAYLGENDIIVATSEKTDSLLRNRTPWLSQITCIVLDEVHLIGSENRGATLEMVITKLRYTNPVMQIIGLSATIGNPAQLAEWLDATLITSTWRPVDLRQGVYYNGKIRFSDSERPIQGKTKHDDLNLCLDTIEEGGQCLVFVSSRRNAEGFAKKAAGALKAGSPDSKALAQELRRLRDRDEGNVLADCVERGAAFHHAGLIRQERTIIEEGFRNGYIEVIAATPTLAAGLNLPARRVIIRDYNRFASGLGMVPIPVGEYHQMAGRAGRPHLDPYGEAVLLAKDAPSVERLFETFIDAEAERVDSQCVDDASLCAHILSLIATGFAHDQEALSSFMERTFYFFQHPKTRSLPRLVADAIRFLTTAGMVEERENTLSATRLGSLVSRLYLNPCTARLILDSLKSCKTPTLIGLLHVICVSPDMQRLYLKAADTQLLRTFLFKHKDDLILPLPFEQEEEELWLSGLKTALVLTDWADEFSEGMIEERYGIGAGDLYNIVDSGKWLLHGTERLVSVEMPEMSQVVKTLSVRVHHGVKSELLPLVALRNIGRVRARTLYNAGYPDPEAVARAGLSTIARIIGEGIARQVIDEITGVKRSGIHSSDDDYQQKTPELLTDIPGIGKKMAEKLQNAGIITVSDLLTADEVLLSDVLGAARARKVLAFLSNSEKENSSSDKTEEIPDTQKIRGQSSWEDFGC

SEQ ID NO: 22 - protein, 1756 aa, Escherichia coli
MMSIAQVRSAGSAGNYYTDKDNYYVLGSMGERWAGKGAEQLGLQGSVDKDVFTRLLEGRLPDGADLSRMQDGSNKHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQAIREAVGEDASLKSRDVAALDTRKSKQHVDPEIRMAEWMQTLKETGFDIRAYRDAADQRTEIRTQAPGPASQDGPDVQQAVTQAIAGLSERKVQFTYTDVLARTVGILPPENGVIERARAGIDEAISREQLIPLDREKGLFTSGIHVLDELSVRALSRDIMKQNRVTVHPEKSVPRTAGYSDAVSVLAQDRPSLAIVSGQGGAAGQRERVAELVMMAREQGREVQIIAADRRSQMNLKQDERLSGELITGRRQLLEGMAFTPGSTVIVDQGEKLSLKETLTLLDGAARHNVQVLITDSGQRTGTGSALMAMKDAGVNTYRWQGGEQRPATIISEPDRNVRYARLAGDFAASVKAGEESVAQVSGVREQAILTQAIRSELKTQGVLGHPEVTMTALSPVWLDSRSRYLRDMYRPGMVMEQWNPETRSHDRYVIDRVTAQSHSLTLRDAQGETQVVRISSLDSSWSLFRPEKMPVADGERLRVTGKIPGLRVSGGDRLQVASVSEDAMTVVVPGRAEPASLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFTELGGEINAQIKRGDLLYVDVAKGYGTGLLVSRASYEAEKSILRHILEGKEAVTPLMERVPGELMETLTSGQRAATRMILETSDRFTVVQGYAGVGKTTQFRAVMSAVNMLPASERPRVVGLGPTHRAVGEMRSAGVDAQTLASFLHDTQLQQRSGETPDFSNTLFLLDESSMVGNTEMARAYALIAAGGGRAVASGDTDQLQAIAPGQSFRLQQTRSAADVVIMKEIVRQTPELREAVYSLINRDVERALSGLESVKPSQVPRLEGAWAPEHSVTEFSHSQEAKLAEAQQKAMLKGEAFPDIPMTLYEAIVRDYTGRTPEAREQTLIVTHLNEDRRVLNSMIHDAREKAGELGKEQVMVPVLNTANIRDGELRRLSTWEKNPDALALVDNVYHRIAGISKDDGLITLQDAEGNTRLISPREAVAEGVTLYTPDKIRVGTGDRMRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVLEPKPDREVMNAQRLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTELAVRDIAGQERDRSAISERETALPESVLRESQREREAVREVARENLLQERLQQMERDMVRDLQKEKTLGGD

SEQ ID NO: 23 - protein, 726 aa, Methanococcoides burtonii
MSDKPAFMKYFTQSSCYPNQQEAMDRIHSALMQQQLVLFEGACGTGKTLSALVPALHVGKMLGKTVIIATNVHQQMVQFINEARDIKKVQDVKVAVIKGKTAMCPQEADYEECSVKRENTFELMETEREIYLKRQELNSARDSYKKSHDPAFVTLRDELSKEIDAVEEKARGLRDRACNDLYEVLRSDSEKFREWLYKEVRSPEEINDHAIKDGMCGYELVKRELKHADLLICNYHHVLNPDIFSTVLGWIEKEPQETIVIFDEAHNLESAARSHSSLSLTEHSIEKAITELEANLDLLADDNIHNLFNIFLEVISDTYNSRFKFGERERVRKNWYDIRISDPYERNDIVRGKFLRQAKGDFGEKDDIQILLSEASELGAKLDETYRDQYKKGLSSVMKRSHIRYVADFMSAYIELSHNLNYYPILNVRRDMNDEIYGRVELFTCIPKNVTEPLFNSLFSVILMSATLHPFEMVKKTLGITRDTCEMSYGTSFPEEKRLSIAVSIPPLFAKNRDDRHVTELLEQVLLDSIENSKGNVILFFQSAFEAKRYYSKIEPLVNVPVFLDEVGISSQDVREEFFSIGEENGKAVLLSYLWGTLSEGIDYRDGRGRTVIIIGVGYPALNDRMNAVESAYDHVFGYGAGWEFAIQVPTIRKIRQAMGRVVRSPTDYGARILLDGRFLTDSKKRFGKFSVFEVFPPAERSEFVDVDPEKVKYSLMNFFMDNDEQ

SEQ ID NO: 24 - protein, 439 aa, unidentified
  source 1..439: note = Enterobacteria phage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 25 - protein, 970 aa, Clostridium botulinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 26 - protein, 94 aa, synthetic construct
  REGION 1..94: note = Synthetic polypeptide
DNQKALEEQMNSINSVNDKLNKGKGKLSLSMNGNQLKATSSNAGYGISYEDKNWGIFVNGEKVYTFNEKSTVGNISNDINKLNIKGPYIEIKKI

SEQ ID NO: 27 - protein, 549 aa, synthetic construct
  REGION 1..549: note = Synthetic polypeptide
ATGGGTCTGGATAACGAACTGAGCCTGGTTGATGGTCAAGACCGTACCCTGACCGTCCAACAATGGGATACCTTTCTGAATGGTGTGTTTCCGCTGGATCGTAACCGCCTGACCCGTGAATGGTTCCATAGTGGTCGCGCGAAATATATTGTCGCAGGCCCGGGTGCTAACGAATTTAGAGGTACGCTGGAACTGGGTTATCAGATTGGCTTTCCGTGGAGCGTTGGCATCAACTTCTCTTACACCACGCCGAACATCAACATCAACAATGGTGACATTACCGCACCGCCGTTTGGCCTGAACTCCGTGATCACGCCGAACCTGTTTCCGGGTGTTAGCATTTCTGCGGGCAACGGTCCGGGCATCCGAGAAGTGGCGACCTTTAGTGTGCGTGTTTCCGGTGCAAAGGGCGGTGTCGCAGTGAGCAATGCACACGGTACCGTTACGGGTGCAGCCGGCGGTGTCCTGCTGCGTCCGTTCGCACGCCTGATCGCTTCAACCGGTGATTCGGTTACCACGTACGGCGAACCGTGGAACATGAATTAATGA

SEQ ID NO: 28 - protein, 849 aa, synthetic construct
  REGION 1..849: note = Synthetic polypeptide
ATGGGTCTGGATAACGAACTGAGCCTGGTTGATGGTCAAGACCGTACCCTGACCGTCCAACAATGGGATACCTTTCTGAATGGTGTGTTTCCGCTGGATCGTAACCGCCTGACCCGTGAATGGTTCCATAGTGGTCGCGCGAAATATATTGTCGCAGGCCCGGGTGCTAACGAATTTAGAGGTACGCTGGAACTGGGTTATCAGATTGGCTTTCCGTGGAGCGTTGGCATCAACTTCTCTTACACCACGCCGAACATCAACATCGACAACGGTGACATCACCGCACCGCCGTTTGGCCTGAACTCCGTGATCACGCCGAACCTGTTTCCGGGTGTTAGCATTTCTGCGGGCAACGGTCCGGGCATCCGAGAAGTGGCGACCTTTAGTGTGCGTGTTTCCGGTGCAAAGGGCGGTGTCGCAGTGAGCAATGCACACGGTACCGTTACGGGTGCAGCCGGCGGTGTCCTGCTGCGTCCGTTCGCACGCCTGATCGCTTCAACCGGTGATTCGGTTACCACGTACGGCGAACCGTGGAATATGAACGATAACCAAAAAGCCCTTGAAGAACAAATGAATAGTATCAACAGCGTGAATGATAAACTTAACAAAGGAAAAGGGAAATTATCTCTTTCAATGAATGGAAATCAACTTAAAGCTACATCTAGCAATGCTGGTTATGGTATCAGTTACGAAGATAAAAATTGGGGTATCTTTGTAAATGGTGAAAAGGTCTATACTTTTAATGAAAAATCAACTGTAGGCAATATCTCTAATGATATTAACAAATTAAACATTAAAGGACCTTATATTGAGATTAAGAAGATCCATCACCATCACCATCACTAATGA

SEQ ID NO: 29 - protein, 546 aa, synthetic construct
  REGION 1..546: note = Synthetic polypeptide
ATGGGTCTGGATAACGAACTGAGCCTGGTTGATGGTCAAGACCGTACCCTGACCGTCCAACAATGGGATACCTTTCTGAATGGTGTGTTTCCGCTGGATCGTAACCGCCTGACCCGTGAATGGTTCCATAGTGGTCGCGCGAAATATATTGTCGCAGGCCCGGGTGCTAACGAATTTAGAGGTACGCTGGAACTGGGTTATCAGATTGGCTTTCCGTGGAGCGTTGGCATCAACTTCTCTTACACCACGCCGAACATCAACATCAACAATGGTGACATTACCGCACCGCCGTTTGGCCTGAACTCCGTGATTACGCCGCCTCTGTTTCCGGGTGTTAGCATTTCTGCGGGCAACGGTCCGGGCATCCGAGAAGTGGCGACCTTTAGTGTGCGTGTTTCCGGTGCAAAGGGCGGTGTCGCAGTGAGCAATGCACACGGTACCGTTACGGGTGCAGCCGGCGGTGTCCTGCTGCGTCCGTTCGCACGCCTGATCGCTTCAACCGGTGATTCGGTTACCACGTACGGCGAACCGTGGAACATGAATTAA

SEQ ID NO: 30 - protein, 849 aa, synthetic construct
  REGION 1..849: note = Synthetic polypeptide
ATGGGTCTGGATAACGAACTGAGCCTGGTTGATGGTCAAGACCGTACCCTGACCGTCCAACAATGGGATACCTTTCTGAATGGTGTGTTTCCGCTGGATCGTAACCGCCTGACCCGTGAATGGTTCCATAGTGGTCGCGCGAAATATATTGTCGCAGGCCCGGGTGCTAACGAATTTAGAGGTACGCTGGAACTGGGTTATCAGATTGGCTTTCCGTGGAGCGTTGGCATCAACTTCTCTTACACCACGCCGAACATCAACATCGACAACGGTGACATCACCGCACCGCCGTTTGGCCTGAACTCCGTGATTACGCCGCCTCTGTTTCCGGGTGTTAGCATTTCTGCGGGCAACGGTCCGGGCATCCGAGAAGTGGCGACCTTTAGTGTGCGTGTTTCCGGTGCAAAGGGCGGTGTCGCAGTGAGCAATGCACACGGTACCGTTACGGGTGCAGCCGGCGGTGTCCTGCTGCGTCCGTTCGCACGCCTGATCGCTTCAACCGGTGATTCGGTTACCACGTACGGCGAACCGTGGAATATGAACGATAACCAAAAAGCCCTTGAAGAACAAATGAATAGTATCAACAGCGTGAATGATAAACTTAACAAAGGAAAAGGGAAATTATCTCTTTCAATGAATGGAAATCAACTTAAAGCTACATCTAGCAATGCTGGTTATGGTATCAGTTACGAAGATAAAAATTGGGGTATCTTTGTAAATGGTGAAAAGGTCTATACTTTTAATGAAAAATCAACTGTAGGCAATATCTCTAATGATATTAACAAATTAAACATTAAAGGACCTTATATTGAGATTAAGAAGATCCATCACCATCACCATCACTAATGA

SEQ ID NO: 31 - protein, 297 aa, Eisenia fetida
MSAKAAEGYEQIEVDVVAVWKEGYVYENRGSTSVDQKITITKGMKNVNSETRTVTATHSIGSTISTGDAFEIGSVEVSYSHSHEESQVSMTETEVYESKVIEHTITIPPTSKFTRWQLNADVGGADIEYMYLIDEVTPIGGTQSIPQVITSRAKIIVGRQIILGKTEIRIKHAERKEYMTVVSRKSWPAATLGHSKLFKFVLYEDWGGFRIKTLNTMYSGYEYAYSSDQGGIYFDQGTDNPKQRWAINKSLPLRHGDVVTFMNKYFTRSGLCYDDGPATNVYCLDKREDKWILEVVG

SEQ ID NO: 32 - protein, 262 aa, Escherichia coli
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES